(19) United States
(12) Patent Application Publication
(10) Pub. No.: US 2025/0334565 A1
(43) Pub. Date: Oct. 30, 2025

(54) Cell Stressor Devices And Methods For Analyzing A Biomechanical Response

(71) Applicant: The Regents of the University of Colorado, a body corporate, Denver, CO (US)

(72) Inventors: Brisa Pena Castellanos, Aurora, CO (US); Orfeo Sbaizero, Boulder, CO (US); Mostafa Abdel-Hafiz, Denver, CO (US); Luisa Mestroni, Boulder, CO (US); Daewon Park, Englewood, CO (US)

(21) Appl. No.: 18/865,835

(22) PCT Filed: May 16, 2023
(86) PCT No.: PCT/US2023/022438

§ 371 Date: Nov. 14, 2024

Related U.S. Application Data
(60) Provisional application No. 63/342,346, filed on May 16, 2022.

(51) Int. Cl. G01N 33/483; C12M 1/42; G01N 3/06; G01N 3/08; G01N 3/22

(52) U.S. Cl. CPC G01N 33/4833; C12M 35/04; G01N 3/068; G01N 3/08; G01N 3/22

(57) ABSTRACT

A cell stressor device for use in analyzing a biomechanical response of cells or tissues may include a body including opposing ledges. A bottom of each ledge may be attached to a bar, and the pair of ledges and the bar may form a window having an opening facing away from a top of the body. The device may include a bender shaped and dimensioned to be inserted into the window. A length and height of the bender may be less than a length and height of the window. The device may include a beam shaped and dimensioned to be inserted into the window between the bender and the ledges to contact the ledges. The device may include means for moving the bender alternately away from and back toward the bar to cause a top of the bender to impinge upon a bottom of the beam.

### CROSS REFERENCE TO RELATED APPLICATIONS

This application claims the benefit of and priority to International Patent Application entitled “CELL STRESSOR DEVICES, SYSTEMS AND METHODS FOR ANALYZING A BIOMECHANICAL RESPONSE OF CELLS OR TISSUES,” filed May 16, 2023 under International Application No. PCT/US2023/022438, which claims the benefit of and priority to a U.S. Provisional Application entitled the same, filed on May 16, 2022 under Application No. 63/342,346, the contents of which are incorporated by reference in their entirety for all purposes.

### BACKGROUND

Pathological mechanical stress is a major cause of cardiac hypertrophy and cardiac remodeling, regulating fibrosis and scar formation. Thus, it is important to understand how mechanical stresses in the heart effect cells under both normal and pathological conditions. Yet there are not proper devices to assess mechanical stresses on cells.

Cardiovascular disease is prevalent in society and is a major target of biomedical research. It is known that cardiac hypertrophy due to mechanical stress of the heart is a part of the progression of heart failure. This can include scarring and fibrosis progression. At least some other types of cells, tissues and organs besides the heart are subject to mechanical stress in their normal or pathological functioning. Mechanoreceptors on cells receive or detect mechanical stress and responsively trigger inter- or intra-cellular biochemical signals, as in transduction pathways. Such signaling pathways may relate to pathology and disease progression. There is an unmet need for tools for modeling molecular mechanisms for how cells respond to changes in their mechanical environment.

Accordingly, a need exists for technology that overcomes the problems demonstrated above, as well as one that provides additional benefits. The examples provided herein of some prior or related devices, systems and methods, and their associated limitations, are intended to be illustrative and not exclusive. Other limitations of existing or prior systems will become apparent to those of skill in the art upon reading the following detailed description.

### SUMMARY

The present technology provides an engineered cell stressor device that can apply tensile and compressive stresses simultaneously on cells. In some embodiments, the cell stressor follows a four-point beam bend as outlined in the American Society for Testing and Materials (ASTM) standard ASTM E855 08. The device may include at least five pieces (main body, bender, beam, nut and bolt). A torque screwdriver may be used to apply and measure the force being applied for providing the stress(es) to the beam. In some embodiments, a motor driven means may be used to apply the torque.

The stress that the cells would experience may be calculated both analytically (using the equations outlined in the ASTM E855 08) and numerically (using finite element analysis). The ability for the torque screwdriver to consistently apply the same amount of force was tested by measuring beam strain. The strain applied on the beam was found to be consistent between samples and matched the expected strain (calculated analytically). To assess the cell stressor, cell culture experiments were performed in which 3T3 fibroblast cells were stressed for 30 minutes (min). Cell migration was observed during the stress assay and quantified using a time-lapse and cell tracking algorithm. Based on nuclei movement and alignment, it was apparent that nuclei of the 3T3 cells nuclei did not try to avoid a particular type of stress; instead, the nuclei aligned and pointed towards the areas of higher stress. This correlated with the post-stress phalloidin staining results in which the cytoskeletons of the cells also were becoming more aligned when compared with cells without stress. Additionally, live/dead staining did not show significant difference regarding cell viability before and after stress. These experimental results confirmed that the cell stressor device according to the present technology was able to apply both tension and compressional stresses on cells and that cells could sense and react to the amount and type of stress being applied by the cell stressor.

A first aspect of the disclosure provides a cell stressor device. The cell stressor device may include a body including a pair of opposing ledges. A bottom of each ledge of the pair of ledges may be attached to a bar. A first side of a first ledge of the pair of ledges may face a first side of a second ledge of the pair of ledges. A second side of the first ledge may face away from a second side of the second ledge. The pair of ledges and the bar may form a window having an opening facing away from a top of the body. The cell stressor device may include a bender shaped and dimensioned to be inserted into the window. A length (Lb) and height (Hb) of the bender may be less than a length (Lw) and height (Hw) of the window. The cell stressor device may include a beam shaped and dimensioned to be inserted into the window between the bender and the pair of ledges to contact the pair of ledges. The cell stressor device may include a means for moving the bender alternately away from and back toward the bar along an axis (A) to cause a top of the bender to impinge upon a bottom of the beam.

In a first embodiment according to the first aspect of the disclosure, at least a portion of a top facing surface of the beam (TFSB) may be configured for culturing of one or more mammalian cell types. In an example of the first embodiment of the first aspect, a width of the device (Wd) may be sufficient to submerge at least a portion of the top facing surface of the beam in a cell culture medium. In another embodiment of the first embodiment, at least a portion of each ledge may be arcuately shaped and dimensioned to facilitate submerging the at least a portion of the top surface of the beam in a container holding the cell culture medium.

In a second embodiment, or in any of the above summarized examples, including the first embodiment, of the cell stressor device according to the first aspect of the disclosure, a value of Lb may be equal to, or substantially equivalent (e.g., within ±10%, or ±5%, or ±1%, or ±0.1%, or ±0.01%) to, a length of the opening (Lo). In a third embodiment, or in any of the above summarized examples, including the first or second embodiments, of the cell stressor device according to the first aspect of the disclosure, the value of Lb may be less than the length of the opening (Lo). In a fourth embodiment, or in any of the above summarized examples, including the first, second or third embodiments, of the cell stressor device according to the first aspect of the disclosure, the value of a length of the beam (LB) may be greater than the value of Lb. In a fifth embodiment, or in any of the above summarized examples, including the first, second, third or fourth embodiments, of the cell stressor device according to the first aspect of the disclosure, the window may be rectangularly-shaped.

In a sixth embodiment, or in any of the above summarized examples, including the first, second, third, fourth or fifth embodiments, of the cell stressor device according to the first aspect of the disclosure, each ledge of the pair of ledges may be L-shaped. In some embodiments, and L-shaped ledge may include a long leg and a short leg. In a first example according to the sixth embodiment, each ledge of the pair of ledges may include a shelf formed on or coupled to a bottom facing surface of the short leg and coupled to a window facing surface of the long leg. In some embodiments according to the first example of the sixth embodiment, the shelf may: extend partially along the window facing surface of the long leg for a first distance less than a value of Hw; and extend partially along the bottom facing surface of the short leg for a second distance less than a second distance of the short leg. In a second example according to the sixth embodiment, when inserted into the window, two portions (P) of a back facing surface of the beam may contact a front facing surface (S) of the shelf of each ledge. In any of the examples of the sixth embodiment of the first aspect, as summarized above, a height of the shelf (Hs) may be less than Hw. In any of the examples of the sixth embodiment of the first aspect, as summarized above, a value of Hw may be less than or equal to a value of a height of the beam (HB). In any of the examples of the sixth embodiment of the first aspect, as summarized above, a top facing surface of the bender may include a pair of nubs coupled to or formed on the top facing surface of the bender. In any of the examples of the sixth embodiment of the first aspect, as summarized above, a back facing surface of the device may include at least one tab coupled to or formed on the back facing surface of the device. In a third example of the sixth embodiment of the first aspect, as summarized above, the means for moving may include a screw. In some embodiments according to the third example of the sixth embodiment, the bar may include a bore axially formed therethrough to receive the screw. In some embodiments according to the third example of the sixth embodiment, the bore and the screw may include matching threads to facilitate securely receiving the screw and moving the bender alternately away from and back toward the bar along an axis (e.g., as by applying a torque to the screw to turn it). In some embodiments according to the third example of the sixth embodiment, the cell stressor device may include an axially aligned nut (e.g., a nut that embodies, or replaces, the bore, or that is positioned in a suitably shaped and dimensioned bore) to provide a portion of the matching threads. In some embodiments according to the third example of the sixth embodiment, the means for moving may include a motor operably coupled to the screw (e.g., directly, or indirectly (e.g., via one or more gears), by way of a motor shaft).

In a seventh embodiment, or in any of the above summarized examples, including the first, second, third, fourth, fifth or sixth embodiments, of the cell stressor device according to the first aspect of the disclosure, the means for moving may include a torque and/or force sensor. In an eighth embodiment, or in any of the above summarized examples, including the first, second, third, fourth, fifth, sixth or seventh embodiments, of the cell stressor device according to the first aspect of the disclosure, the means for moving may be configured to cause the beam to undergo both compression and tension. In some embodiments, the beam being caused to undergo both compression and tension may thereby cause cells and/or tissues cultured on, or otherwise attached to, the beam to experience corresponding stresses.

In a ninth embodiment, or in any of the above summarized examples, including the first, second, third, fourth, fifth, sixth, seventh or eighth embodiments, of the cell stressor device according to the first aspect of the disclosure, the means for moving may include a motor. In a tenth embodiment, or in any of the above summarized examples, including the first, second, third, fourth, fifth, sixth, seventh, eighth or ninth embodiments, of the first aspect, one or more of the body, the bender, the beam, and the mean for moving may be fabricated at least in part using a 3D printing process.

A second aspect of the disclosure provides a system for analyzing a biomechanical response of cells and/or tissues. In some embodiments of the second aspect, the system may be configured to, or otherwise capable of, analyzing biomechanical responses of live cells and/or tissues. The system according to the second aspect may include a plurality of cell stressor devices according to any of the embodiments and examples of the first aspect of the disclosure, as summarized above.

A third aspect of the disclosure provides a method for analyzing a biomechanical response of cells and/or tissues using the cell stressor device or the system according to any of the embodiments and examples of the first, or the second aspect, of the disclosure, as summarized above. The method according to the third aspect may include the step of culturing cells on the beam submerged in the cell culture media. The method may include the step of moving the bender to impinge on the beam. The method may include the step of identifying biophysical effect(s) of the moving step. The moving step may include causing the cultured cells to undergo both compression and tension.

In a first embodiment of the method according to the third aspect of the disclosure, the identifying step may include imaging the cells. In an example of the first embodiment of the third aspect, the imaging step may include: first imaging the cells before the moving step; and second imaging the cells after the moving step.

In second embodiment of the method according to the third aspect, or in any of the above summarized embodiments or examples thereof, the method may include the step of assembling at least one of the cell stressor device. In an example of the second embodiment of the third aspect, the method may include the step of sterilizing at least a portion of the cell stressor device or the system according to the first, and the second, aspects of the disclosure, respectively. In some embodiments, at least one of the assembling step and the sterilizing step may be performed before the culturing step of the method.

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are, references to the same embodiment; and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Through this disclosure, references to external documents listed herein are numbered in single brackets “[ ]”, and such references are made using the corresponding number in the single brackets, in the same manner as those sources are listed herein. The entire contents of each of the listed documents are incorporated herein by reference in their entireties.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

One or more embodiments of the present invention are illustrated by way of example and not limitation in the figures of the accompanying drawings, in which like references indicate similar elements.

FIG. 1 illustrates a process of cardiac remodeling following a cardiac injury.

FIG. 2 illustrates a process a cell undergoes to migrate.

FIG. 3 illustrates a flow chart for generating a variomap from a binary image.

FIG. 4 illustrates atomic force microscopy (AFM) in an experiment. First the cantilever approaches, depresses the cell and then retracts. This results in a force curve (right), with one line representing the approach and the other line representing the retraction, from where the Young's Modulus can be calculated at the contact point.

FIG. 5 illustrates the difference between a confocal and conventional fluorescent microscope images. Panel a shows the confocal microscope image, panel b shows the conventional microscope image. The individual actin fibers are more visible in the confocal microscope image than the conventional microscope image.

FIG. 6 illustrates the 4-point beam bend described in ASTM E855 08 [61].

FIG. 7 shows pictures of the first design of the cell stressor. At left is an image of the device in use. At right is a top down view of the assembled cell stressor.

FIG. 8 illustrates a system including the cell stressor device according to the present technology, and with the cell stressor device in use.

FIG. 9 illustrates the pattern used for laser cutting the beams. 320 beams were able to fit on one 30 cm square sheet.

FIG. 10 provides a visual representation of the image stitching algorithm. The first image from the left is the starting image and the following images show the image slowly growing as more images are stitched together. This is followed by the cell detection algorithm to highlight cells (blue). Scalebar is 400 μm.

FIGS. 11A-11D illustrate examples of a cell stressor device and an associated system and method according to some embodiments of the present technology.

FIG. 12 illustrates a finite element analysis (FEA) model showing the stresses present on the beam upon application of 85N of force. The maximum stress at the top of the beam was 28 MPa and the minimum stress at the bottom of the beam was −73 MPa.

FIG. 13 illustrates a comparison between the before and after stress distance between the etches. For the unstressed control beam there were no differences found between the two images. For the first sample both the top and bottom were significantly different under stress (p>0.0001). The second sample both the top and bottom of the beam were significantly longer and shorter respectively (p>0.001, p>0.0001). The third sample both the top and bottom of the beam were significantly longer and shorter respectively (p>0.001, p>0.0001).

FIGS. 14A-14C illustrate strain quantification: FIG. 14A is the before, and FIG. 14B is the after, pictures of a stressed beam along the 2 score marks. FIG. 14C is the quantification of the strain at the top and bottom of the beam the top of the beam (tension) was in strain for all samples except the control. n=25, p>0.0001. The bottom of the stressed beams were in compression and were significantly different than the control p>0.0001.

FIG. 15 illustrates a visual representation of the methods used to prepare the beams and preform the tests.

FIG. 16 illustrates (a). the unprocessed image taken on an evos fluorescence microscope. (b). The k-means clustering of the row image. (c). The edge detection method of segmenting the cells. (d). The final segmented image highlighting the detected cell area in blue.

FIG. 17 illustrates a live and dead stain of the bottom (compression), middle (neutral) and top (tension) of the beam showing that the stress is not affecting cell viability.

FIG. 18 illustrates the test image used along with the five images for the different methods of edge detection. The sobel, prewitt and roberts methods were underestimating where the cells were located. The Canny and log methods were both overestimating where the cells were located with the log method showing less overestimation than the canny method.

FIG. 19 illustrates the test image used along with the five images for the different methods of edge detection using half of the threshold automatically identified. The sobel, prewitt, canny, and log methods were all severely overestimating where the cells were. The roberts method was only slightly underestimating the area covered by the cells.

FIG. 20 illustrates examples of how the different parameters used for the texture based approach identified the cells. These representative images show how the kmean algorithm varies as the parameters were changes, While there were many parameters that correctly identified the cells (bottom left and top right) the one that was chosen was the one with the lowest parameters as that took less processing time.

FIG. 21 illustrates the same image run through the algorithm twice right most image is the masks subtracted.

FIGS. 22A and 22B illustrate the migration analysis using a nuclear stain. In FIG. 22A, the first and last image of the time lapse preformed using a nuclear stain. The subtracted binary image shows the differences between the “first image” and the “last image”. FIG. 22B provides quantification of the nuclei migration/alignment showing significant differences in nuclei movement/alignment towards between the different groups. In the stress group top is tension, bottom is compression, and middle is neutral. p>0.0001.

FIGS. 23A and 23B illustrate phalloidin staining along with the visualization of filament orientation using 2-D autoclustrograms. In the stressed samples top and bottom there is a clear major axis in the center of the variomap indicating that the actin fibers are aligning. In the stressed middle there is no clear major axis but there are a couple starting to occur. In all the control samples there is no major axis indicating that the is no alignment of the actin fibers.

FIG. 24 illustrates the improved beam design. On the left is the adobe illustrator design that was replicated to fill an entire PPMA sheet on the right is an example of a laser cut improved beam design.

FIG. 25 illustrates beam renders for use with gels. The top panel show a Solidworks® render of a beam with the middle rectangle completely removed. the middle panel show a render of just the top 1 mm removed creating a well. The bottom panel shows a render of the middle segment completely removed.

FIG. 26 illustrates a brightfield mage of the cells pre and post stress showing some movement.

FIG. 27 illustrates the omega ring concept. This design can have either tension or compression by rotating the shape.

FIG. 28 illustrates AFM results showing significant differences between the stiffness of the stressed sample and the control.

FIG. 29 illustrates examples of variomaps generated from sample binary images. First row shows lines that are perfectly vertical. The resulting variomap's center shows a vertical orientation. Second row shows lines that are perfectly horizontal. The resulting variomap's center shows a horizontal orientation. Last row shows lines that are angled. The resulting variomap's center shows the same angle as the lines in the binary image.

FIG. 30 is a diagrammatic representation of a machine, in the example form, of a computer system within which a set of instructions, for causing the machine to implement or otherwise perform any one or more of the techniques and methodologies of the present technology described herein, may be executed.

FIG. 31 illustrates a cell stressor device according to embodiments of the present technology.

FIG. 32A are images showing phalloidin, paxillin, and DAPI staining on female and male cells under no stress, compressional, and tensional stresses with a scale of 33 μM.

FIG. 32B provide cell count shown as folds over control showing control, compressional stress, and tensional stress in females and males.

FIG. 32C provides paxillin-positive area percent normalized based on cell count for control, compressional, and tensional stresses in females and males.

FIG. 33A provide images of SMA and DAPI staining on female and male fibroblast cells under no stress, compressional, and tensional stresses with scalebar of 30 μM.

FIG. 33B provides quantification of the SMA intensity comparing the female and male fibroblasts cells in each category (no stress, compression, and tension).

FIG. 34 provides atomic force microscopy results depicting the Young's Modulus for control, compression, and tension for both females and males, as well as statistical analysis.

FIG. 35A provides confocal images of phalloidin, paxillin, and DAPI staining on recovery beams with scale of 33 μM.

FIG. 35B provides cell count in recovery beams for controls, compressional stress, and tensional stress in females and males.

FIG. 35C provides paxillin-positive area percent normalized based on cell count for controls, compressional, and tensional stresses in females and males.

FIG. 36A depicts Piezo1 ion channel staining images on the confocal with scale of 33 μM.

FIG. 36B provides Piezo1-positive area percent versus each category for females and males (control, compression, and tension).

FIG. 37 depicts the microscope images taken of the female and male fibroblasts under no stress as well as compressional and tensional stresses.

### DETAILED DESCRIPTION

The heart is a contractile mechanical organ that pumps nutrient carrying blood around the body. In humans, the heart beats about 100000 times per day and over 2.5 billion times in a lifetime [1].

With every beat, the heart experiences large changes in stress and tissue strain [2].

With changing physiological conditions, the heart adapts its output to meet demand. In order to adapt cardiac output, the heart undergoes intrinsic mechanisms [3] at the cellular, tissue, and organ level to generate and respond to mechanical stress and regulate cardiac function [4] under both physiological and pathological conditions. Under pathological conditions, the heart remodels to try and sustain cardiac output. This pathological remodeling can lead to heart failure (HF).

The public health burden of heart failure: HF is a major public health problem in the United States and around the world [5]. It is one of the leading causes of hospitalizations for adults and the elderly. An estimated 5.7 million people in the United States live with HF with about 870,000 cases diagnosed every day. HF incurs costs of ˜$25K per patient per year and >$30B annually [6] in the US (direct costs; projected to >$60B by 2030) [7, 8]. The current treatments for HF are aimed at reducing symptoms, slowing disease progression, and reducing mortality and not aimed at repairing heart muscle or restoring function [9]. Furthermore, even with these treatments, approximately half of patients with HF will die within 5 years of diagnosis [10, 11]. Cardiac transplantation remains the only definitive treatment for those affected with end-stage HF, but the availability of donor hearts remains a major limitation [12, 13]. While there is an ongoing effort to create therapies to regenerate the myocardial tissue and prevent fibrosis, there is currently no cure.

Prevalent causes of heart failure are cardiac pathologies, such as those caused by cardiac injury or by cardiomyopathies. Nearly all patients affected by cardiac pathologies develop cardiac fibrosis, which involves pathological myocardial remodeling characterized by excessive deposition of extracellular matrix (ECM) proteins, particularly type I and II fibrillar collagens [14]. The over-production of ECM increases tissue stiffness, which affects the physiological mechanics of the heart, results in impairment of cardiac contraction and relaxation and leads to cardiomyocyte (CM) loss and progression towards HF [15-19].

### Cardiac Cells Interactions

The heart is composed of different types of cells such as cardiomyocytes, fibroblasts, macrophages, etc. The cell population in the ventricular regions of the heart (apex, interventricular septum, left ventricle) is composed of 49.2% cardiomyocytes, 21.2% mural cells, 15.5% fibroblast, 7.8% embryonic cells, and 5.3% immune cells [20].

Cardiac development, maintenance, and remodeling of the cardiac tissue involves several cell-cell and cell-ECM interactions [21]. Recent studies have placed an increasing amount of emphasis on understanding how cardiac cells communicate under physiological and pathological conditions. This may occur via chemical, electrical, and mechanical signaling. Disruption of any of these signals, such as that caused by mechanical stress, alters the heart homeostasis [21]. Under pathological conditions, for example after cardiac injury, there are dramatic shifts in the various cardiac cell populations that can alter cardiac function [22]. Cardiac fibroblasts are a key component in normal heart function and during the remodeling process through dynamic cell-cell interactions and synthesis and degradation of the ECM. For example, following a cardiac injury, a period of cardiac remodeling occurs. This remodeling can lead to regenerated cardiomyocytes or the formation of a cardiac scar [23]. However adult mammalian cardiomyocytes have a very limited regeneration capacity, and thus, a fibrotic scar is formed.

In the following detailed description of certain embodiments, reference is made to the accompanying drawings which form a part hereof, and in which are shown by way of illustration of example embodiments. It is also to be understood that features of the embodiments and examples herein can be combined, exchanged, or removed, other embodiments may be utilized or created, and structural changes may be made without departing from the scope of the present disclosure.

In accordance with various embodiments, the methods and functions described herein may be implemented as one or more software programs running on a computer, processor, or controller. Dedicated hardware implementations including, but not limited to, application specific integrated circuits, programmable logic arrays, system-on-chip (SoC), circuit logic, and other hardware devices can likewise be constructed to implement the circuits, functions, processes, and methods described herein. Methods and functions may be performed by modules or engines, both of which may include one or more physical components of a computing device (e.g., logic, circuits, processors, controllers, etc.) configured to perform a particular task or job, or may include instructions that, when executed, can cause a processor to perform a particular task or job, or may be any combination thereof. Further, the methods described herein may be implemented as a computer readable storage medium or memory device including instructions that, when executed, cause a processor to perform the methods.

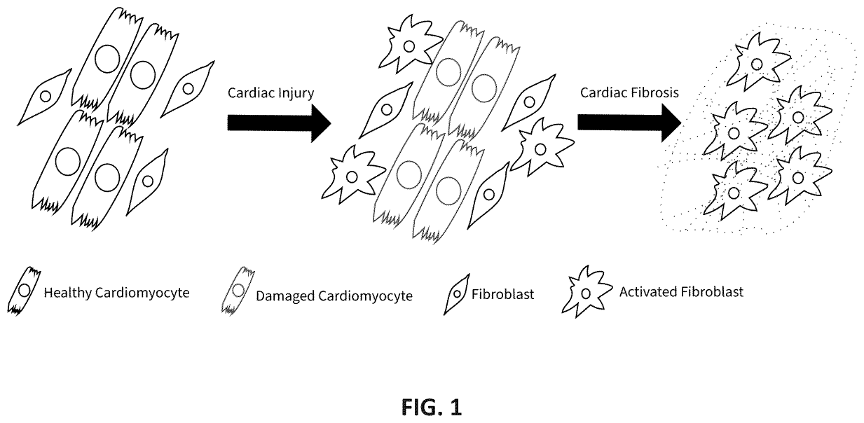

FIG. 1 shows the process of cardiac remodeling following cardiac injury in which cardiomyocytes are lost and fibroblasts become activated forming scar tissue to compensate. The cells responsible for the fibrotic scar formation are myofibroblasts.

Myofibroblasts are a cell type that exhibits the characteristics of both fibroblasts and smooth muscle cells [24, 25]. One of their main roles post-cardiac injury is the secretion of ECM [26]. Additionally, myofibroblasts exhibit contractile activities that also help reshape the heart. Initially, this is a beneficial process as it prevents the heart from rupturing. However, the fibrotic scar is stiffer than the surrounding tissue and can lead to a forward feeding cycle that leads to more fibrosis within the heart [27]. With increasing fibrosis in the heart, the mechanical properties change significantly leading to ineffective cardiac function. In addition, fibrosis also impairs electrical conduction in the heart leading to arrhythmias [28]. The combination of these factors inevitably leads to heart failure.

Myofibroblast can be transdifferentiated from multiple cell sources including resident fibroblasts, smooth muscle cells, vascular pericytes, endothelial and epithelial cells, and circulating fibrocytes [27, 29]. While their exact source in the adult heart remains largely unknown. It is thought that local fibroblasts play a role in both transdifferentiation and recruitment of cells to the site of injury [30]. Their transdifferentiation is driven by both chemical and mechanical stimuli. While chemical signaling is well studied, the role of mechanical stresses is still a growing field.

### Mechanosensing and Mechanotransduction

Mechanosensing refers to a cell's ability to sense its mechanical environment. This includes sensing the stiffness of a substrate and stresses present around them. Mechanotransduction refers to the cell's ability to convert mechanical stimuli into electrochemical signals. Currently, there is no single theory as to what is sensing the mechanical environment.

The stiffness of an object can be defined as the object's ability to resist deformation when a force is applied. This is quantified by the Young's Modulus or elastic modulus

$$E = \frac{σ}{ε} ;$$

where E is the modulus, σ is stress and ε is strain.

The stiffer an object is, the higher the Young's Modulus will be, which means that it will be more resistant to deformation. In cardiac tissue upon fibrosis, the tissue becomes stiffer and more resistant to deformation leading to a higher Young's Modulus.

Strain is the proportional deformation of an object it is defines as

$$ε = \frac{Δ L}{L} ;$$

where ε is strain, ΔL is change in length and L is length.

Stress is the amount of force on a given area and is defined as

$$σ = \frac{F}{A}$$

where σ is stress, F is force and A is area.

With every beat, the heart undergoes deformation and experiences stress. A couple of the types of stress that the heart experiences are compressive and tensile stresses. Compressive stresses are when the forces are perpendicular to the material pushing toward the center of the material causing the material to compress. Tensile stresses are when the forces are perpendicular to the material and pull away from the center causing the material to stretch. Currently there is no single theory as to what is sensing the changing mechanical properties and stresses in the heart. However, the tensegrity theory of mechanosensing implicates several structural proteins and the ECM network [31].

### ECM in the Cardiac Tissue

The main components of the ECM are structural proteins, such as collagen and elastin, and non-structural proteins, such as proteoglycans, proteases, and growth factors. From a mechanical standpoint, the most important of these proteins is collagen which is primarily found as collagen I and collagen III. Other collagen types, such as collagen IV, V, and VI are also present in the ECM. The primary purpose of collagen, in the healthy myocardium, is to prevent overstretch, provide surfaces for myocytes to attach and help in transmitting contractile forces to the cardiac tissue. Other important ECM proteins are fibronectin, which is involved in regulating the assembly of collagen I, and elastin, which forms long and thin elastic fibers surrounded by microfibrils and proteoglycans which regulate water content [4].

The mechanical cues of the ECM act as a driver for a number of cell functions including differentiation, motility, fibroblast activation, and collagen production [32]. Mechanical properties of the ECM can change drastically during physiological and pathophysiological conditions (e.g. mechanical overload). Under physiological conditions, the structural stability of the ECM is thought to offer, to the cardiac cell population, protection from any substantial change due to the mechanical stresses involved in the beating heart. Under pathological conditions ECM plays an important role in cardiac remodeling, for instance after a cardiac injury, a stable scar must quickly form to prevent ventricular wall rupture [33].

### Cytoskeleton and Mechanical Sensing

The cytoskeleton is a dynamic network of interlinking protein filaments and it is composed of microtubules (MTs), microfilaments (MFs), and intermediate filaments (IFs). Because of its role in cell shaping, migration, and intracellular architecture conditioning, the cytoskeleton is involved in many cellular functions. Mechanical signaling originates from receptor-mediated nucleation of adhesions structures, and further organization of intracellular cytoskeleton with consequent force generation (from intracellular or external stimuli) and intracellular mechanical transduction. For example, connections between the cytoskeleton and the external ECM, through focal adhesion contacts and multi-protein structures which connect the actin bundles to the ECM, generate pathways of mechanotransduction, that are cascades of events originating from mechanical cues eventually leading to biological responses.

### Cytoskeleton and Mechanical Sensing

Cell migration is an important cellular process that is essential for development, immune response, and disease [34] (such as fibroblast migration post cardiac injury). Whether the cells are migrating as a collective or as individual cells, there is usually a stimulus guiding that movement. The process of cells moving in response to a stimulus is called “taxis”. The stimuli can be chemical, electrical, or mechanical, and the responsive movements are called chemotaxis, haptotaxis, and durotaxis respectively. While chemotaxis has been studied since the late 1800s it was not until 2000 that durotaxis was studied [35, 36]. In the initial study, Lo et al showed that 3T3 fibroblasts migrated from areas of low stiffness to areas of higher stiffness [37]. The group was able to achieve this by using collagen-coated polyacrylamide. To vary the stiffness, the team kept the acrylamide concentration the same and varied the bis-acrylamide concentration. They would then add a drop of the stiff solution next to a drop of the soft solution. This created a stiff side and a soft side to the polyacrylamide. They then observed single cells found in the transition region between stiff and soft [37]. Since then, several other studies have tried similar approaches with different cell types and have found that the preference toward stiffer or softer environments depends on the cell type [36]. Additionally, it was shown that some cell types have no preference as single cells but as clusters of cells show significant preferences.

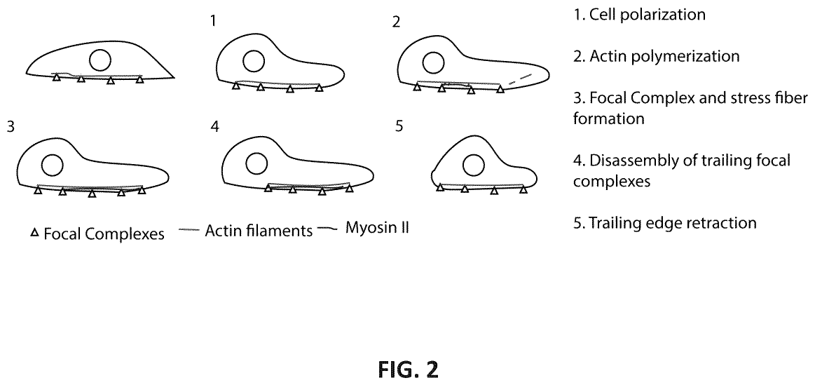

There are many proteins involved in the process of cell migration. The main structures are actin filaments, focal complexes and myosin II. Actin filaments provide the structure in the migrating cell, focal complexes provide anchor points of the cell and myosin II is a motor protein that provides contractile activity. In a simplified model of cell migration (FIG. 2), cells go through a few steps. The cell becomes polarized with a leading and trailing edge (FIG. 2.1). In the leading-edge, actin filaments polymerize extending the cell membrane (FIG. 2.2). The actin filaments then connect to focal complexes anchoring the cell to the substrate. The actin filament then associates with myosin II to form stress fibers and transfer contractile force to the focal contacts (FIG. 2.3). In the trailing edge, focal adhesions disassemble (FIG. 2.4). This allows the trailing edge to retract and the cell moves toward the leading edge [38] (FIG. 2.5).

### Goal of the Study

Cardiac cells are dynamic physical structures that constantly encounter mechanical stimuli in their environment [39], such as dynamic forces and mechanical features of the ECM. These mechanical cues are transduced into biochemical signals and integrated with genetic and chemical signals to modulate cardiac homeostasis. Although it is well known that mechanical stresses sensed by cells result in a broad variety of cellular responses [40], the molecular mechanisms underlying this ability, the mechanoreceptors which receive mechanical stress and convert it into intracellular biochemical signals, and how cardiac cells interact with each other under pathological and physiological conditions are still poorly understood. This is in part attributed to the lack of proper in vitro systems that can recapitulate cardiac injuries, such as tension and compressional stresses that occur with cardiac damage and recovery [41]. Thus, a greater discernment of these mechanisms is fundamental to better understand the development and progression of various cardiac pathologies in which a dysregulation of the mechanosensing mediated pathways is involved.

The long-term goal of this project is to engineer a cell stressor that mimics the stresses that happen in the heart and its environment under both pathological and physiological conditions. For this, it was necessary to first work on the design of a functional cells stressor and to show that cells can in fact detect and react to the magnitude and type of stress being applied by this device. Thus, cell migration, cytoskeleton changes and cell stiffness were the parameters selected to assess the function of the cell stressor, as further discussed below.

### Literature Review of the Techniques and Methodologies Used

### Cell Selection (3T3 Fibroblast)

3T3 cells were chosen for a few key reasons. As previously mentioned, 3T3 cells could detect and react to the stiffness of the material they were cultured on. They were also shown to be a relatively fast-moving cell. Additionally, the cells proliferate quickly allowing for an easier culture start up. 3T3 cells are also a well-established cell line which are easier to culture than primary cells and are less prone to contamination than a primary cell derived fibroblast colony. 3T3 cells were originally established using Swiss albino 17-19 day mouse embryos [42]. The method that was used originally established this cell line was to stick to a rigid transfer schedule. After initial extraction of the fibroblasts from the embryo, the cells were transferred every 3 days at and plated at a 300000 cells per 50 mm Petrie dish. The line was considered established upon a return of the initial growth rate. Later, this process was repeated using NIH Swiss mouse embryos and the NIH-3T3 cell line was established.

### Image Analysis

### Image Color Space

To start with digital image processing, a basic understanding of how color images are digitally represented is needed. One of the ways to represent a color image is to use the red, green, blue (RGB) color space. An image in the RGB color space is a stack of matrixes with each matric representing the relative amount of color. With the first matrix being red, the second matrix being green and the third matrix being blue. By separating the color channels, any non-greyscale image can be segmented into three grayscale images that can then be treated as a grayscale image. This can help reduce computing time when only one of the colors is of interest. In addition to the RGB representation of the color space, there are several other that can be used including hue, saturation, value (HSV), YCbCr and L*a*b* [43]. Each one of these color spaces represents a different way to numerically represent colors and might be useful in different scenarios. For example, if working with an immunohistochemistry stained image and using more than the three different colors (red, green, blue) to color the image, an HSV color space might be more useful to separate out the colors since the first value is HSV is the hue of the color.

### Image Segmentation

Image segmentation refers to the process of separating the foreground/object of interest from the background/rest of the image. In the biological sciences, this is oftentimes trying to separate cells from the rest of the image. There are many different ways that this can be achieved. Some of these methods rely on setting up the experiment ahead of time to get better results.

Segmentation can be achieved through machine learning [44, 45] by training a neural network using a database of existing segmented images and having the computer identify characteristics that separate background from cells. This process needs massive amounts of data to avoid overtraining the network at identifying a certain cell type and can also be very demanding in terms of computing power [45]. Another disadvantage is that you might not know what features are being used to segment the image. Currently, this is used in self-driving cars where objects are quickly identified using a massive amount of data to train the network [46]. This process is usually not perfect and relies on high frame rates and sensor data to reliably identify objects.

Other less intensive approaches include color thresholding and texture analysis. There are many different filters aimed at using small differences in pixel brightness to segment cells or detect the texture. This includes the sobel, laplace, and garbor filters just to name a few [47, 48].

The use of fluorescent markers on the cells can make the process of segmenting the cells much easier. By selecting one color channel that the cells will be brightest in, the process of segmenting the cells from the background because a simple threshold filter. This removes all pixels that are lower than a certain brightness and leaves the bright pixels behind [49].

### Cell Tracking

One method of assessing migration is through the use of a microscope and cell tracking. Cell tracking is used to track individual cells of clumps of cells through the field of view. One way that this is done is through the use of time-lapse imaging. In time-lapse imaging an image is taken on a set interval of the same field of view. Using the time-lapse cell tracking is usually divided into a few steps. The first step is identifying the cells from background (segmentation), The second step is correlating cells from one frame to the next (tracking step). There are several different methods and approaches to each of these steps. Recently there has been a rise in the use of machine learning to try and achieve both steps.

For shorter time-lapses a few assumptions can be made. One being that the cells are not moving large distances, the cells are not frequently dividing, and a non-significant number of cells are leaving the frame, although this can be combated by either excluding the cells on the border or tracking them until the leave and marking them as no longer in the frame.

An additional method that is used in motion detection outside the cell field is subtractive imaging [50]. This relies on the fact the camera is stationary between consecutive frames. Then, the two images are subtracted from one another. Assuming that the brightness was held relatively consistent between frames, the resulting image should have the brightest pixels be the moving object [50]. If there was a major adjustment to the brightness of the images between frames, this can be accounted for by calculating the mean brightness of both images and adjusting the images to have the same mean brightness. This can then be followed by the subtraction step that shows differences.

### Two Dimensional (2-D) Autocorrelation

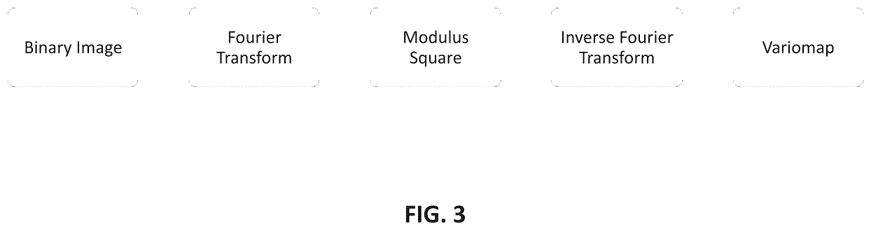

2-D autocorrelation is a method used to produce variomaps [51]. In binary images a variomap describes the probability of encountering a white pixel from another white pixel. As such it can be used to summarize a binary image and revels directionality in an image. This algorithm relies on preforming a Fourier transform on an image followed by the modulus square and a reverse Fourier transform (FIG. 3). Originally this algorithm was developed for analyzing pores in rocks and extracting directionality and size of the pores in an automated fashion [51]. It was recently used to test the alignment of the cytoskeletal structure in cells after being exposed to stress [52]. This technique is depicted in FIG. 3, which is a flow chart of a method for generating a variomap from a binary image.

### Atomic Force Microscopy (AFM)

Atomic force microscopy (AFM) is a type of scanning probe microscopy. AFM was first invented in the late 1980s by Gred K. Benning with a proprietary patent filed in November 1985 [53] and the first paper outlining the machine was published in 1986 [54]. The AFM was developed as an alternative to the scanning tunnel microscope (STM) and relies on many of the same principles to operate. The STM operates by using a very sharp tip and bringing the tip really close to a sample so that the electron clouds of the tip and the material are touching. A tunnel of current is then free to flow between the tip and sample. The current is exponentially dependent on the distance between the sample and the tip and thus the topography of the sample can be measured at an atomic scale [54]. This method however had the limitation of needing to be used on conductive materials and coating the surface with a conductive material has the drawback of losing resolution [53]. To overcome this limitation, the AFM was used where the tip of the STM does not come in contact with the sample, instead, a spring is used to touch the sample and the deflections were originally picked up using the STM. The deflection of the cantilever is now most commonly measured by a laser and photodiodes.

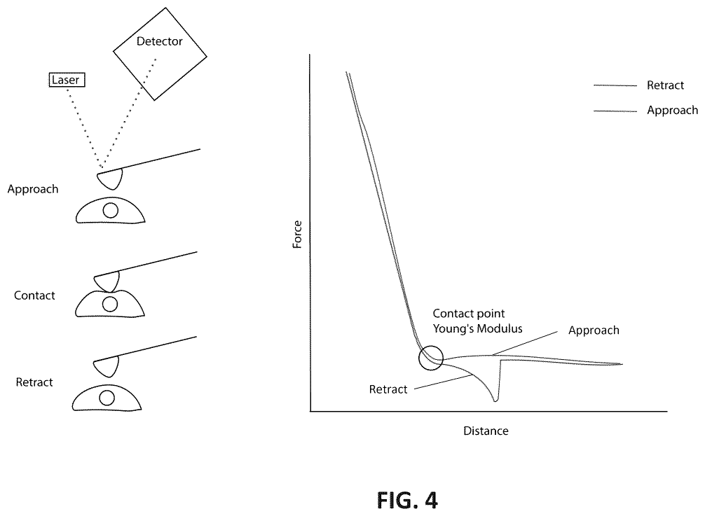

AFM can be used to measure mechanical properties of cells. For this, there are several models, but the most popular is force spectroscopy which is a contact mode that performs force-distance measurements. In force spectroscopy, lateral position is set at a fixed point, thus the cantilever moves vertically towards and away from the surface [55]. As the tip is pushed against the sample, mechanical properties values can be measured from indentation (FIG. 4). This generates a force-distance curve. This force-distance curve can then be used to calculate the relative Young's Modulus of the cell using the Hertz method.

FIG. 1. AFM Force spectroscopy. First the cantilever approaches, depresses the cell and then retracts. This results in a force curve (right), with one line representing the approach and the other line representing the retraction, from where the Young's Modulus can be calculated at the contact point.

### Cell Staining

Cell staining refers to a set of techniques used to introduce a dye or florescent molecule to a cell. The goal of cell staining is to better visualize different cells or different components of the cell. There are many types of cell staining techniques and each relies on a different mechanism to selectively stain the target [56]. For this project, live/dead and phalloidin were used as cell staining to assess both cell viability and changes in the cytoskeleton.

Live/dead staining relies on 2 different chemicals to stain live and dead cells. The live cells are usually stained green and use a cell membrane-permeable dye. While the dead cells are usually stained red and rely on a dye that is unable to cross the cell membrane of a living cell. One of the most common dies used in live staining is calcian acetoxymethyl [57]. This is initially a non-fluorescent molecule and relies on the esterase activity in live cells to hydrolysis the ester group. This leaves behind calcian which is not a cell-permeable molecule and is fluorescent. Ethidium Homodimer-1 is one of the dyes used to stain the dead cells [57]. Once the cell membrane of dead/dying cells starts to degrade, the dye can enter the cell and bind to the DNA within the cell. While this dye is weakly fluorescent before binding to DNA, after binding it becomes more fluorescent and can be detected with a fluorescent microscope. Hoechst stains are another nuclear stain that uses bis-benzamides. This molecule is cell-permeable and is able to cross the membrane of live cells. Once inside the cell, it binds to DNA and fluoresces blue.

For imaging actin in cells, phalloidin staining was used. This is similar to an antibody stain where the actin filaments are targeted, however the phalloidin molecule is a peptide instead of an antibody that has a high affinity for actin. Most often this peptide is conjugated to a fluorescent molecule and can be imaged using fluorescence microscopy.

### Confocal Microscopy

Confocal microscopy was designed by Marvin Minsky in 1955 [58]. This microscopy method is most often used in 3-D samples or thicker samples or for imaging samples at a higher resolution. With conventional microscopy, only a thin segment can be imaged cleanly. This is due to the method of illumination. With conventional microscopy, the entire field of view is illuminated and while the brightest light can be found in the focal point of the microscope there are areas above and below the focal point that remain illuminated [59]. This causes out-of-plane fluorescence to degrade the image. The confocal microscope solves this problem in two ways. First, the light is focused on a smaller area in the sample, this prevents some of the background from other segments being illuminated. In addition, confocal microscopes have a secondary pinhole before the detector [60]. This pinhole is in a conjugate focal plane from the tissue so only the light coming from the focal plane passes through and all other light is blocked. Both of these combined means that only a small plane of the sample is imaged at any given time. This eliminates background from different planes and allows for sharper-looking images. It also allows for imaging of thicker samples and focusing on only one plane at a time. This also means that a thicker sample can be imaged one plane at a time which results in z-stack images.

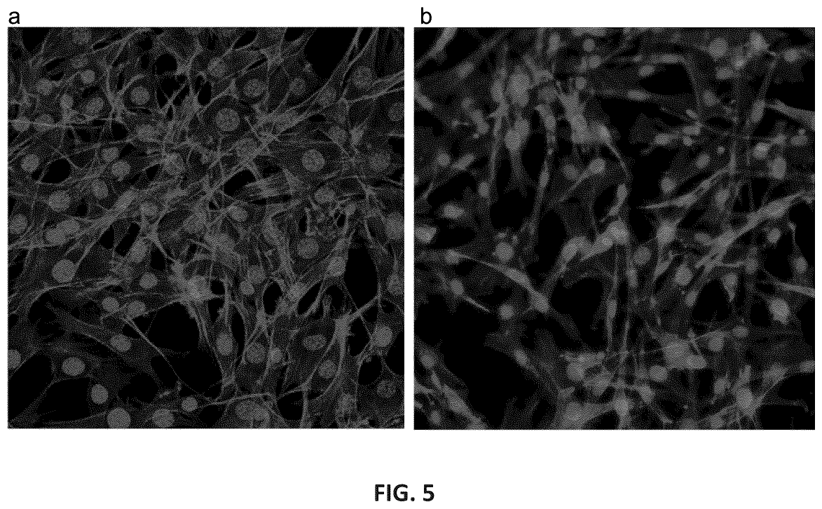

Fluorescence microscopy can be used in combination with a confocal microscope to obtain sharper images of a cell stain. The key difference between fluorescence microscopy and brightfield (normal) microscopy is the use of florescent labels. In fluorescence microscopy, a specific frequency of light is used to excite the florescent label. One way to achieve this is passing the light through a filter before it reaches the sample. The filter allows only the desired frequency of light to pass. When the light hits the sample, it excites a fluorescent molecule that then re-emits light at a different frequency. This is then detected by the detector. In the case of a confocal microscope, the light passes through a pinhole before being detected. FIG. 5 shows the difference between using a conventional microscope and confocal microscope to image a phalloidin stained sample. Panel “a” shows the confocal microscope image and panel “b” shows the conventional microscope image. The individual actin fibers are more visible in the confocal microscope image than the conventional microscope image.

### Cell Stressor Design

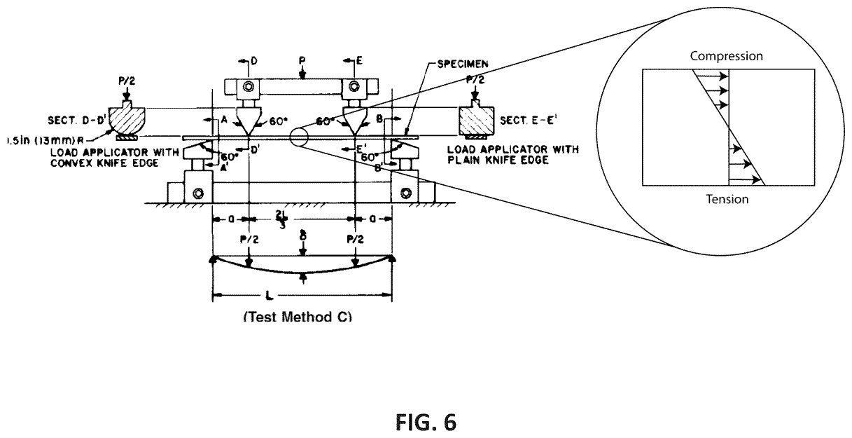

The cell stressor device according to the present technology adopts the ASTM four-point beam bend, including “Test Method C” thereof (FIG. 6). This is a technique used to test material properties. The advantage of having the 4-point beam bend is that there is both compressive and tensile stresses present with a transition point between the compressive and tensile stressed. FIG. 6 depicts the 4-point beam bend described in ASTM E855 08 [61].

### Example 1

### Hypothesis

As previous studies have shown, cells are able to react to mechanical stimuli. As such, we hypothesized that tensile and compressive stresses will affect cellular function cues such as cell migration, cell morphology and cell biophysics.

### Specific Aims

### Aim 1: Engineer a Functional Cell Stressor

The first step of this project is to modify the prototype design of the cell stressor to make the device functional for assessing the effects of stresses on cells. As previously mentioned, there were shortcomings in the device that made it complicated to use and cells were not able to attach properly, making the device unfunctional. These shortcomings will be addressed and basic tests will be performed to characterize the stress being applied on cells.

### Aim 2: Evaluate the Effect of Compressional and Tension Stresses on Cells

3T3 cells will be used to evaluate the new design of the cell stressor and to assess the effect of compressional and tension stresses on cells. Particular interest will be focused to engineer a cell tracking algorithm to assess cell migration under the stressed condition. Furthermore, cytoskeleton staining will be used to determine stress-caused changes in the cell morphology. AFM will be also used to assess the effect of mechanical stresses on the mechanical properties of the cells.

### Methods for Aim 1

### Device Design and Fabrication

The device was composed of five parts: 1) the main body, which was designed to fit in a 60 mm culture dish and provides the two outer supports of the 4-point bend; 2) the bender, which is the segment that receives the force and had the two loading point for the 4-point bend; 3) the nut and 4) the bolt, which both provide a method of applying the force to the bender and, 5) the beam, which is where cells are cultured and which is the one being subjected to mechanical stresses.

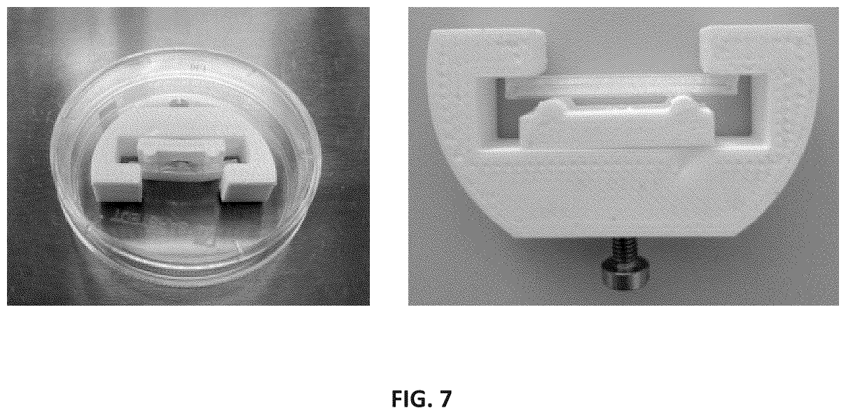

The first attempt, made by our collaborators, of the cell stressor was not very successful. Polylactic acid (PLA) was used to three-dimensional (3D) print the main body, bender and beam of the first design. The nut and bolt were made of titanium. To apply the force a torque screwdriver was used. FIG. 7 shows an image of the first design of the cell stressor. At left is an image of the device in use, and at right is a top down view of the assembled cell stressor.

While the initial device was a good base, there were some shortcomings. The main issue with the first cell stressor was that the cells were difficult to seed on the beam. This was because 1) the initial cell stressor was made to be fully assembled before seeding the cells on the beam; and 2) the cells were only intended to be cultured on the small area between the two-point of the bender. With such a small area to culture cells, it was difficult to keep the cells contained on the material. Additionally, if the medium were to touch the bender, the cells would be lost due to capillary action causing the entire solution to run down the bender. With minimal amounts of medium being used, the medium evaporating was also an issue. With the device needing to be fully assembled before seeding the cells, the number of tests that can be run in a single day with the same device was also limited. Another issue is the choice of material for the beam. While 3D printed PLA is a good material to start with for testing, the cells would get caught in the ridges created by the additive printing process. Additionally, PLA is a ductile material and will undergo plastic deformation which needed to be avoided. This investigation further improved the cell stressor with the objective to make it functional and useful to assess stresses on cells.

As previously mentioned, the device design was based on a 4-point bend test as outlined in the standard ASTM E855 08. This standard test method outlines a material strength test that is used for metallic flat materials. Solidworks® was used to model the improvements made on the cell stressor. The improved cell stressor was 3D printed using acrylonitrile butadiene styrene (ABS). The beams were laser cut out of polymethyl methacrylate (PMMA) with the protective film still attached (to prevent material fogginess) and the nut and screw were stainless steel.

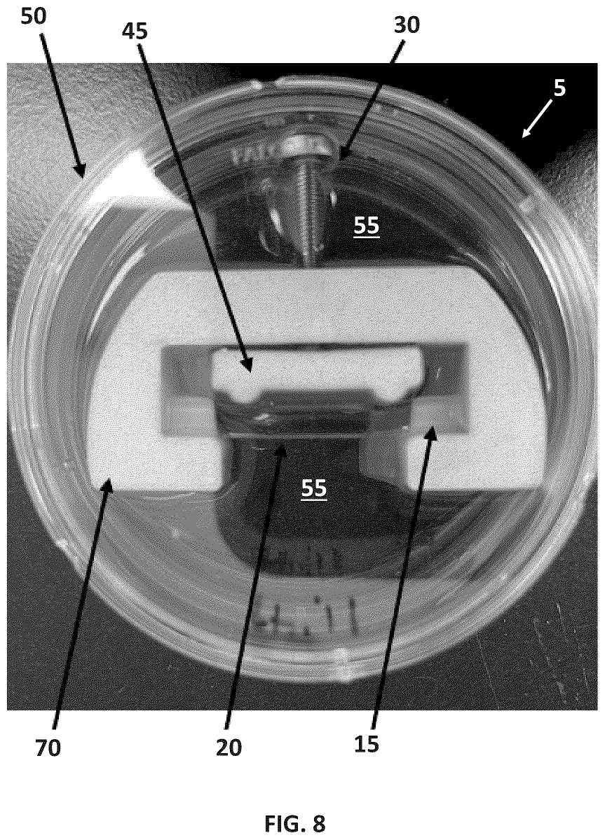

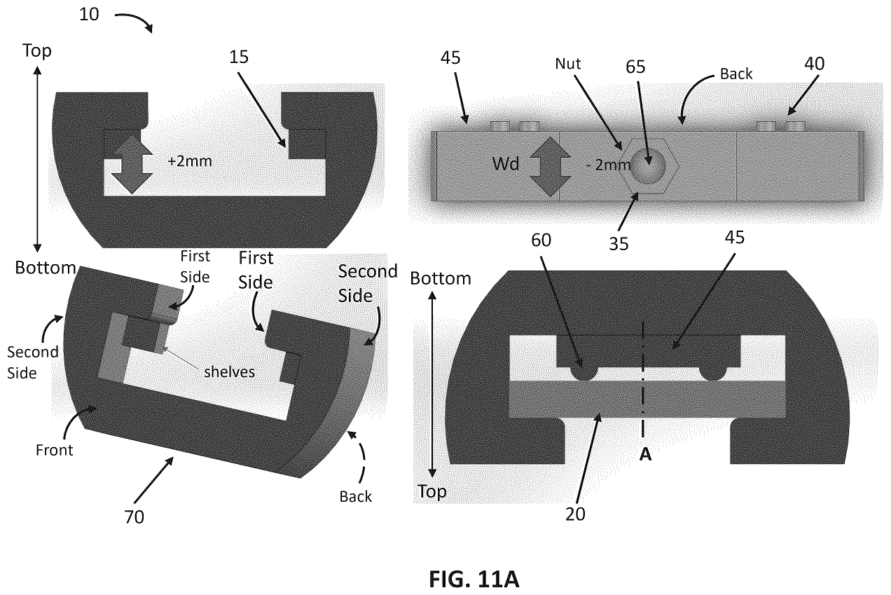

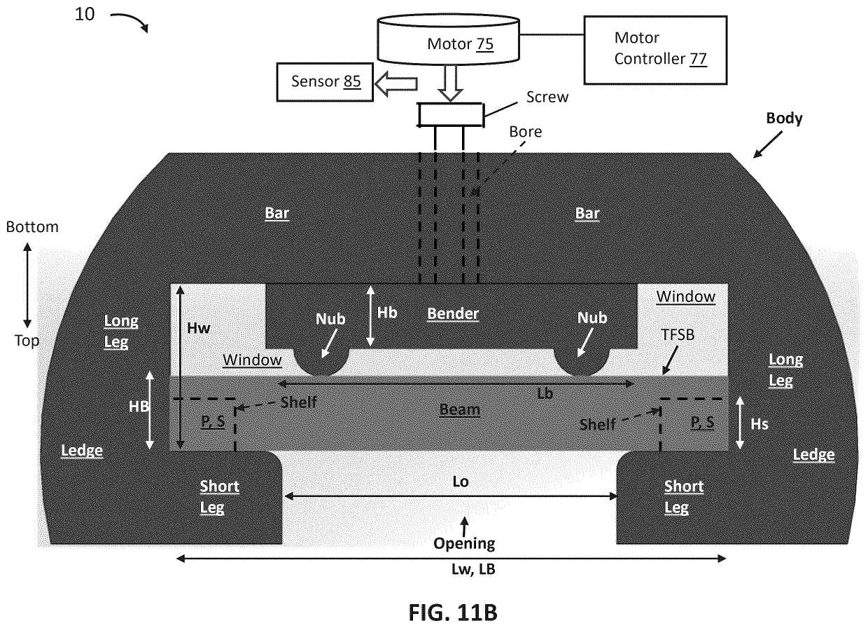

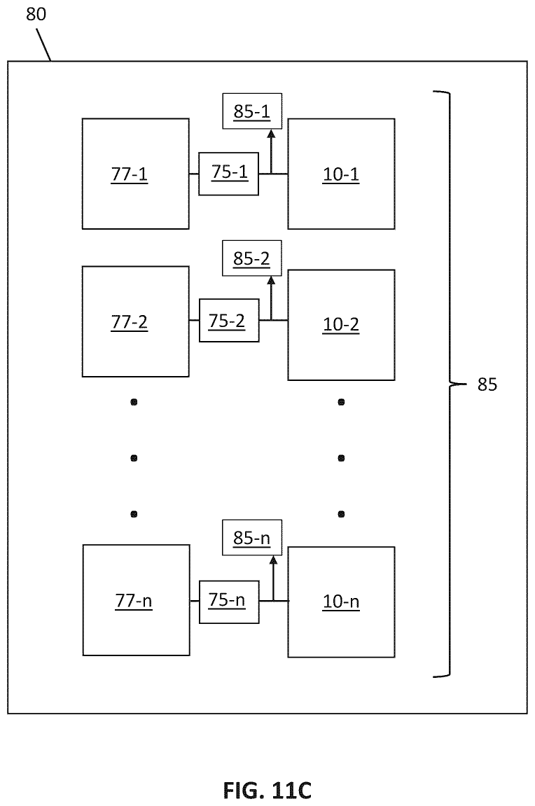

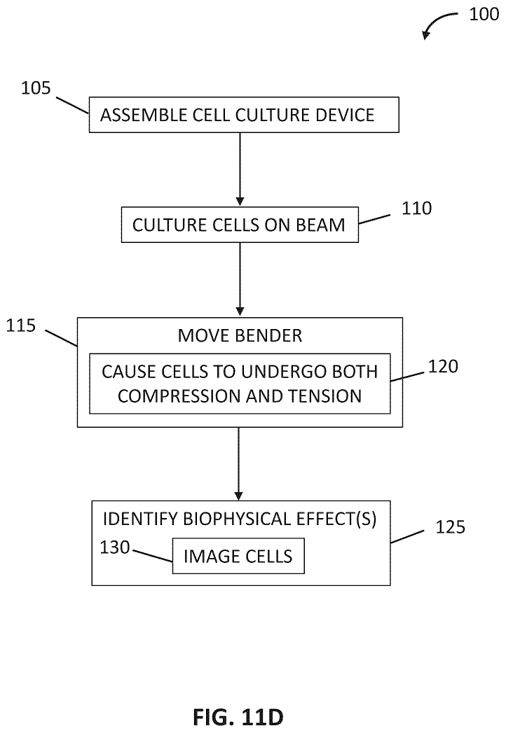

An improved cell stressor system (5) is shown in FIG. 8. System (5) includes the cell stressor device (10) at least partially submerged in cell culture media (55) such that the beam (20) is immersed in the media (55). The major components are labeled in FIG. 8. Major improvements include the addition of shelves (15), wider area for the beam (20) and shorter cell stressor. These improvements are discussed more with reference to FIG. 11 and in the results section.

FIGS. 11A-11D illustrate embodiments of devices, systems and methods relating to the cell stressor device (10) according to the present technology. With additional reference to FIG. 8, in FIG. 11A, the top left panel shows the increase in device (10) width; the top right panel shows the decrease in device (10) height; the bottom left panel show the addition of shelves (15) to the device (10) to support the beam (20); and the bottom right panel show the assembled device (10) excluding the screw (30) and nut (35). Tabs (40) on one or both sides of the bender (45) enable space to be introduced between the bottom of the petri dish (50) and the cell stressor device (10), which may facilitate circulation of media (55), gas exchange, consistent regulation of temperature, and other physical and/or chemical effects that may be beneficial to the cell culture for the uses described herein. A pair of nubs (60) are coupled to, or formed in or on, a side of the bender (45) opposite the threaded bore (65), where the bore (65) does not extended entirely through the bender (45), and where as the screw (30) is inserted through main body (70). The nubs (60) provide for a uniform force to the applied, as described herein. In some embodiments, the threaded bore (65) is provided by the nut (35), where a corresponding space to securely accommodate the nut (35) is formed in a side of bender (45) opposite the side having the nubs (60).

With reference to FIGS. 11A-11D, in some embodiments, a cell stressor device (10) includes a main body (referred to herein also as “body”) including a pair of opposing ledges. A bottom of each ledge of the pair of ledges is attached to a bar. A first side of a first ledge of the pair of ledges faces a first side of a second ledge of the pair of ledges, and a second side of the first ledge faces away from a second side of the second ledge. The pair of ledges and the bar form a window having an opening facing away from a top of the body. The cell stressor device example shown in FIGS. 11A and 11B includes a bender shaped and dimensioned to be inserted into the window. A length (denoted “Lb”) and height (denoted “Hb”) of the bender are less than a length (denoted “Lw”) and height (denoted “Hw”) of the window. The cell stressor device (10) may include a beam shaped and dimensioned to be inserted into the window between the bender and the pair of ledges to contact the pair of ledges. Cell stressor device (10) may include means for moving the bender alternately away from and back toward the bar along an axis (denoted “A”) to cause at least a portion of a top of the bender to impinge upon a bottom of the beam.

In some embodiments, at least a portion of a top facing surface of the beam (denoted “TFSB”) is configured for culturing of one or more mammalian cell types. In this respect, the at least a portion of the top surface of the beam may be treated or functionalized to enable cells to be cultured thereon. In an example, a width of the device (denoted “Wd”) is sufficient to submerge at least a portion of the top facing surface of the beam in a cell culture medium. As shown in FIG. 11B, at least a portion of each ledge may be arcuately shaped and dimensioned to facilitate submerging the at least a portion of the top surface of the beam in a container holding the cell culture medium (e.g., petri dish).

As shown in FIG. 11B, Lb may be equal to a length of the opening (denoted “Lo”). Lb may less than Lo. A length of the beam (denoted “LB”) may be greater than Lb. The window may be rectangularly-shaped. Each ledge of the pair of ledges is L-shaped may include a long leg and a short leg. Each ledge may include a shelf formed on or coupled to a bottom facing surface of the short leg and coupled to a window facing surface of the long leg. In some examples, each shelf may extend partially along the window facing surface of the long leg for a first distance less than Hw, and each shelf also may extend partially along the bottom facing surface of the short leg for a second distance less than a second distance of the short leg. When inserted into the window, two portions (denoted “P”) of a back facing surface of the beam may contact a front facing surface (denoted “S”) of the shelf of each ledge. A height of the shelf (denoted “Hs”) is less than Hw. Hw may be less than or equal to a height of the beam (HB).

In some embodiments, a top facing surface of the bender may include a pair of nubs coupled to or formed on the top facing surface of the bender. A back facing surface of the device (10) may include at least one tab coupled to or formed on the back facing surface of the device.

In some embodiments, the means for moving may include a screw (30). In an example, the bar may include a bore axially formed (e.g., along axis A) therethrough to receive the screw (30). The bore and the screw (30) may include matching threads to facilitate securely receiving the screw and moving the bender alternately away and back toward the bar along axis A. The cell stressor device (10) may include an axially aligned (e.g., along axis A) nut (35) to provide a portion of the aforementioned matching threads. In an example, the portion of the matching threads may be half of the matching threads.

Embodiments of the cell stressor device (10) may include an electric motor (75), either instead of, or in addition to, screw (30). Motor (75) may replace, or supplemental, a screw driver in some embodiments. Motor (75) may be operatively coupled to a motor controller (77). In an example, motor (75) may be operably coupled to the screw (30), where for example and without limitation, a shaft of motor (75) may controllable rotate in clockwise and/or counterclockwise directions to alternatively spin screw (30) and thereby facilitate the moving 115 step of method 100, as described below with reference to FIG. 11D. In one example, motor (75) may be directly coupled to screw for torque transfer. In another example, motor (75) may be operably coupled to screw (30) by way of suitable intermediate devices, as in gear(s) and/or a transmission. Similarly to as described herein for use of torque sensing screwdrivers, device (10) may include a torque and/or force sensor (85). Such sensor(s) may be coupled to a computer or processor subsystem to enable a user of device (10) and/or system (80) to perform various operations such as data analysis based on sensor (85)-acquired data.

In some embodiments, the means for moving according to the present technology may be configured to, or otherwise capable, of causing the beam to undergo both compression and tension. In an example, one or more of the body, the bender, the beam, and at least a portion of the mean for moving is fabricated at least in part using a 3D printing process.

With reference to FIG. 11C, one or more cell stressor device(s) (10) may be configured for use in a larger system (80) for use in screening biomechanical responses of cells or tissues using the cell stressor device(s) (10) and related techniques according to the present technology. In the example shown in FIG. 11C, the system (80) for analyzing a biomechanical response of cells or tissues may include a plurality (85) of cell stressor devices (e.g., 10-1, 10-2, . . . , 10-n) according to any of the herein described and illustrated embodiments of the device (10) according to the present technology.

FIG. 11D provides a flowchart of a method (100) for analyzing a biomechanical response of cells or tissues using any of the cell stressor device (10) and/or system (80) embodiments described herein according to the present technology. One or more steps of method (100) may be partially or entirely performed by a robot and/or a computer. Method 100 may include the step of assembling (105) the cell stressor device (10). Device (10) may be submerged at least partially in the media after the assembling (105) of method (100). Assembling (105) the device (10) may be performed, at least in part, after the beam is provided with cells and/or material on one or more surfaces of the beam.

Method (100) may include the step of culturing (110) cells on the beam submerged in the cell culture media. Method (100) may include the step of moving (115) the bender to impinge on the beam. In some embodiments, the moving (115) step comprises causing (120) the cultured cells to undergo both compression and tension. As used herein, the terms “culturing” or “culture” may refer to one or more actions or processes intended to grow or keep alive cells or tissues outside of a host organism (e.g., in vitro). Method (100) may include the step of identifying (125) biophysical effect(s) of the moving (115) step.

The identifying (125) step of method (100) may include the step of imaging (130) the cultured cells. In some embodiments, imaging (130) the cultured cells may include first imaging the cells before the moving (115) step, and the second imaging the cells after the moving step.

### Beam Preparations

In order to promote cell attachment to the beam material, a 0.1% gelatin coating was added on top of the material. Briefly, we first start preparing the gelatin solution in which 500 mL of deionized water were added to a 500 ml glass bottle and then 0.5 grams of gelatin were added to the water. The solution was then autoclaved at 250° C. for 10 minutes, allowed to cool down, filtered using a 0.2 μm filter and stored at 4° C.

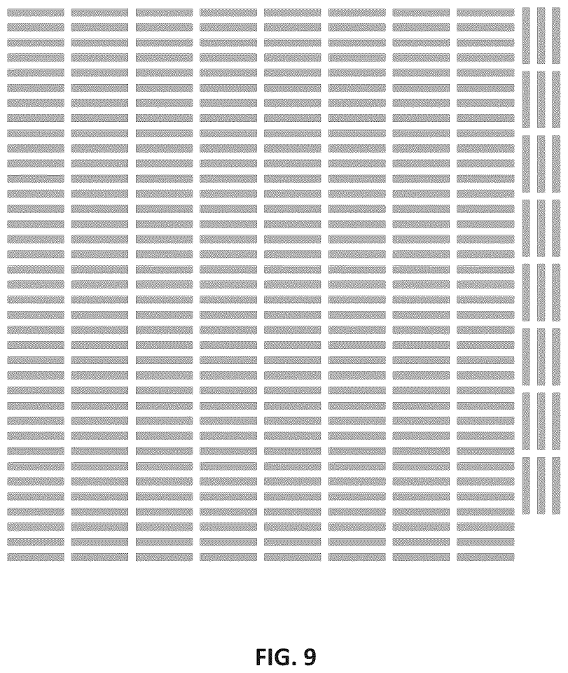

Prior to gelatin coating, the beam was scored along the middle using a razor and then placed in a 60 mm cell culture dish and placed under UV light for half an hour per side to sterilize the material. Following the hour sterilization period, 8 ml of 0.1% gelatin was added to the culture dish, in order to coat the beams. The beams in gelatin solution were incubated at 37° C., 5% CO2 overnight. The gelatin was aspirated the following day and the beams were left to dry in the incubator overnight. The following day the culture dish was sealed with parafilm and placed in the fridge for use the following week. FIG. 9 shows the laser cutting pattern that was used to make the beams. The spacing was kept at 4 mm between each beam to prevent beams from fusing together. 320 beams were able to fit on one 30 cm square sheet.

### Test Protocol

The main device, bender nut, and bolt were first sterilized under UV for 1 hour. Then, the nut was placed in its hole and the bolt was screwed in the nut such that the first threads were visible above the nut. The bender was then placed with the intend on the screw. 9 mL of culture medium were then added to a 60 mm cell culture plate along with the device. A beam was then added to the device ensuring that it is seated flush with the shelves on the main device. For the experimental group, 5.1 Ncm of torque was applied using a Checkline TSD digital torque screwdriver (5-50 Ncm range, ±2% error).

### Image Stitching

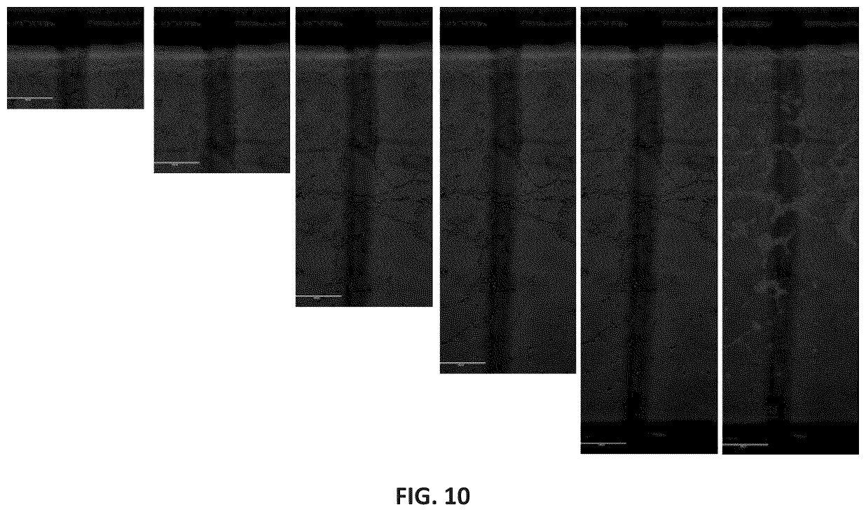

In order to have images of cells across the entire beam, five to six images taken along the width of the score mark were stitched together using a MATLAB algorithm. Using the MATLAB cor2 command, a correlation was run between the last 20-pixel rows and the first 20-pixel rows of the second image. This process was iteratively repeated using the first 20-pixel rows of the second image and gradually moving toward the first-pixel row of the first image. This created a vector of correlation values between the first 20-pixel rows of the second image and the entirety of the first image. The maximum correlation value was found using the max command along with the corresponding row index. The first image was then copied into a new variable up to the index of maximum correlation and the second image was copied into that same variable starting with the index of maximum correlation. This created an image that was a stitch between the first and second images. To add the rest of the images, this stitched image was considered the first image and the second image was the next image that needed to be added. This process was repeated until all the images were combined into a single image. FIG. 10 shows the process gradually adding images to the first image, The last image is the highlighted cells (blue). FIG. 10 provides a visual representation of the image stitching algorithm. The first image from the left is the starting image and the following images show the image slowly growing as more images are stitched together. This is followed by the cell detection algorithm to highlight cells (blue). The scalebar in FIG. 10 is 400 μm.

### Strain Analysis

The strain present in the beam was analyzed to ensure that cells in every experiment were subjected to similar stresses. The first step was adding a second score mark to the bottom of the beam in order to have two reference points to calculate the strain on the beam. The device was set up as per testing protocol (without cells) and horizontal images along the two scored lines were captured using an EVOS fluorescence microscope. The images were then stitched together using the image stitching algorithm described previously. The process of selecting the four corners of the rectangle made by the two score marks and the beam boarders was done manually. Each corner of the rectangle was selected 25 times in a round robin fashion starting from the bottom left most corner. The distance between the bottom corners and between the top corners was calculated. To minimize bias, the beam that was viewed was randomized and the order of stress or unstressed was also randomized.

### Statistical Analysis

All analysis were done using MATLAB and results were presented as means±standard deviation. Student t-tests were used for comparison between 2 groups and ANOVA was used for comparisons between multiple groups. Tukey's honestly significant difference procedure was used to determine significance between multiple groups. A p-value of less than 0.05 was considered significant.

### Results and Discussion for Aim 1

### Device Design and Analysis

As above mentioned, the first cell stressor had a few shortcomings. The overall design of the device was kept similar but with a few key changes. The device was made shorter, the gap in the middle of the device was made wider and shelves were added to the inside as shown in FIG. 11.

FIG. 2. Major changes to the device. Top left panel shows the increase in device width, top right panel shows the decrease in device height, bottom left panel show the addition of shelves to the device to support the beam, bottom right panel show the assembled device excluding the screw and nut.

The device was made shorter so that it can better fit into a 60 mm petri dish and be covered with medium without spilling during transport. Shelves were added so that the beam can be added later after the cells were cultured. The device was made wider to accommodate for the wider beam.

PMMA was chosen as the material for the beams as it was shown to be biocompatible, it is optically clear and it is brittle. Additionally, PMMA can be bought commercially with consistent mechanical properties and thickness. This also provided a smooth surface that the cells can be cultured on. Transparency was important for imaging. Having a brittle material was also important as it means while the beam is under load there will not be any plastic deformation. The smooth surface was important so that the cells could not be caught in any dips and free to migrate. A torque screwdriver along with a nut and bolt were kept as the method of applying force to the system.

The desired force to be applied was around 30 MPa. This was used along with the 4 point bending equations to calculate a range of what the torque screwdriver would measure. Based on these calculations, a Checkline TSD digital torque screwdriver was chosen. This screwdriver has a working range of 5-50 Ncm of torque and a ±2% error. To ensure device function and gain preliminary results, the lowest measurable torque was used throughout the project. The equations below (derivation in Appendix B) show how much stress was analytically calculated on the beam. In addition to the analytical calculations, a finite element analysis in Solidworks® was used to visualize the stress that would be present on the beam. For the simulation, the main body was assumed to be fixed, the bender was assumed to only slide up and down along one plain and 85 N of force were applied to where the screw would sit.

$$F = \frac{T}{c D} ;$$

where F is force, T is torque, c is coefficient of friction (0.2), and D is bolt diameter.

$${σ}_{x} = \frac{FL}{2 {bh}^{2}} ;$$

wherein L is distance between supports, b is thickness, h is height, and σₓ is the stress.

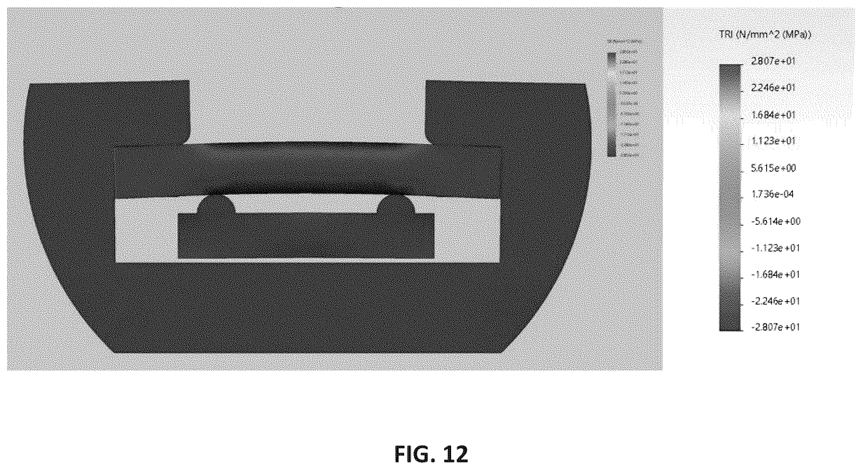

The finite element analysis was successfully run using the method outlined above. The maximum stress found in the beam was 28 MPa (FIG. 12). The minimum stress on the beam was −73 MPa, however this was localized to the segment of the beam in immediate contact with the bender. Near the center of the beam, where experiments were performed, the stress was −28 MPa as expected. Using the equations outlined above the stress was found to be 26 MPa. While the stresses were not exactly matching, they were in the same magnitude. Another way of confirming that the stresses were being applied as calculated and consistently every time would be to use the strain on the beam and use the Young's Modulus of the beam to confirm the expected strain.

FIG. 3 provides an FEA model showing the stresses present on the beam upon application of 85N of force. The maximum stress at the top of the beam was 28 MPa and the minimum stress at the bottom of the beam was −73 MPa.

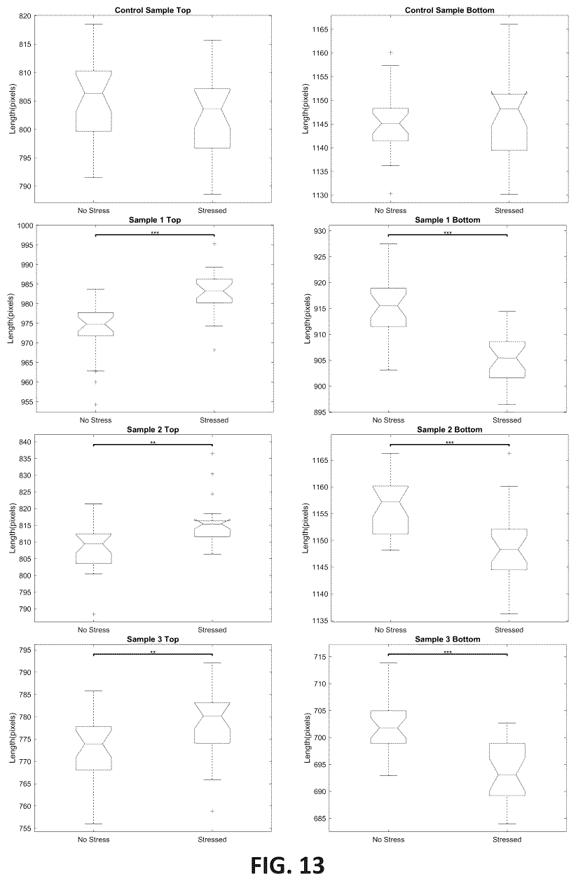

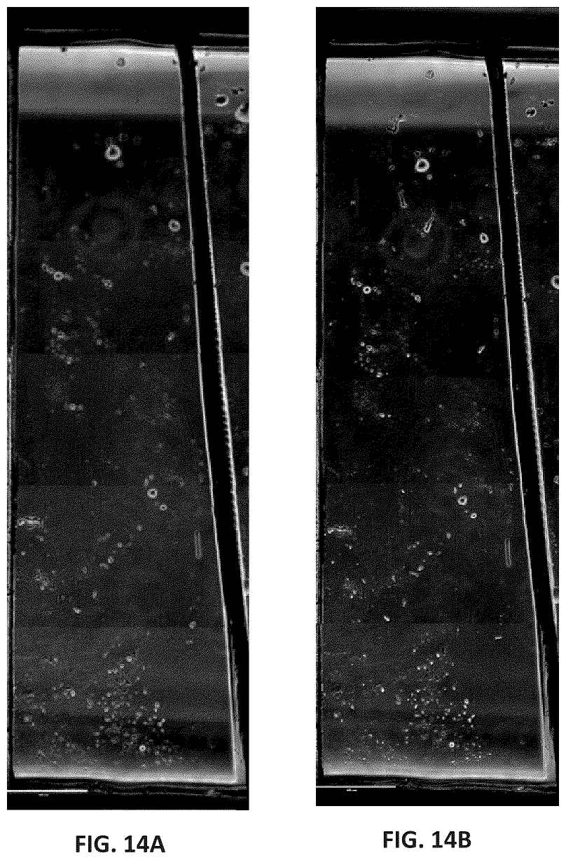

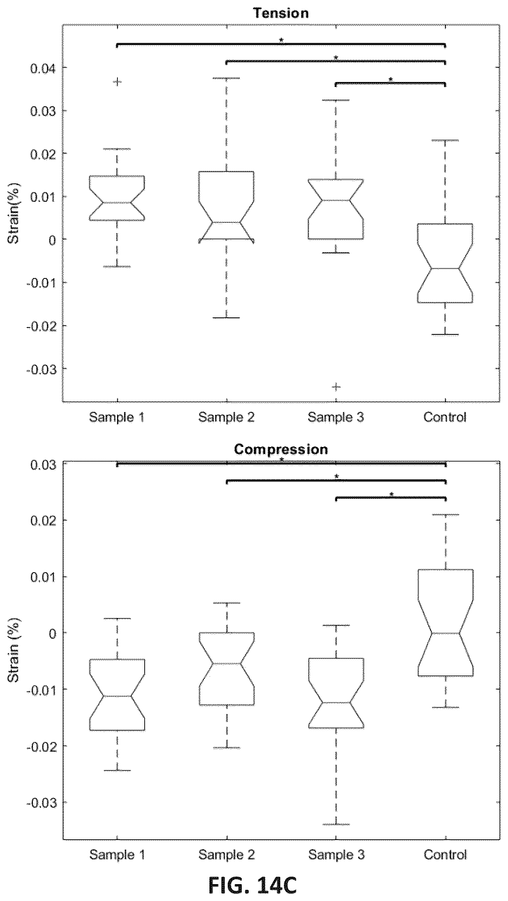

The experimentally applied strain was successfully calculated in the beams. A t-test was run on lengths measured before and after the stress was applied. The samples under stress were significantly different than before the stress was applied (p>0.05). The control sample with no stress applied showed no statistical significance (FIG. 13). The strains of the stressed samples were compared and there was no statistical difference between all three stressed samples (FIG. 14C). The three stressed samples were however significantly different from the control sample. The average strain for the stressed samples seen was about 0.0085 and −0.0107 in tension and compression respectively. Using the Young's Modulus of the beams (E), obtained from the manufacture's site, the experimentally applied stress was calculated as follows.

$$E = \frac{σ}{ε}$$

Starting with the equation for calculating the Young's Modulus, where E is the modulus, σ is stress and c is strain. In this equation the stress in the unknown variable and can be solved for by multiplying both sides by the stain.

$$E * ε = σ$$

This results in the equation where all the known variables are on one side and the unknown can be calculated. E is equal to 2.5 GPa, ε was experimentally calculated to be 0.0096.

$$23. MPa = 2.4 * GPa * 0.0096$$

This resulted in an experimentally calculated strain of 23 MPa. This was slightly lower than the expected stress on the beam. The error could have been due to several factors including increased friction between the nut and bolt, slight changes in the beam dimension or the Young's Modulus of the beam being slightly different than what the manufacture claimed. However, since there was no statistical difference between the sample, it was confirmed that using the torque screwdriver to measure the torque being applied was a reliable method of estimating the force being applied onto the beam.

FIG. 4 depicts a comparison between the before and after stress distance between the etches. For the unstressed control beam there were no differences found between the two images. For the first sample both the top and bottom were significantly different under stress (p>0.0001). The second sample both the top and bottom of the beam were significantly longer and shorter respectively (p>0.001, p>0.0001). The third sample both the top and bottom of the beam were significantly longer and shorter respectively (p>0.001, p>0.0001).

FIGS. 14A-14C illustrate strain quantification: FIG. 14A is the before, and FIG. 14B is the after, pictures of a stressed beam along the 2 score marks. FIG. 14C depicts the quantification of the strain at the top and bottom of the beam the top of the beam (tension) was in strain for all samples except the control. n=25, p>0.0001. The bottom of the stressed beams were in compression and were significantly different than the control p>0.0001.

### Aim 2—Evaluate the Effect of Compressional and Tension Stresses on Cells

### Methods for Aim 2

A 1 ml vial of 3T3 cells were rapidly thawed in a 37° C. for 2 minutes. The cells were then transferred to 9 ml of complete medium (10% bovine calf serum and 1% penicillin-streptomycin to Dulbecco's modified eagle medium) and spun down at 900 RPM for 6 minutes. The cells were then resuspended in 10 ml of complete medium and added to a T75 cell culture flask. The cells were split every 3 days.

Cells were manually counted using a hemocytometer and clinker counter. Gelatin coated-beams were placed in a 40 mm culture dish and 3T3 cells were seeded on top of the beams with a density of 14000 cells per beam. The cells were suspended in 100 μL of medium and added dropwise to the beam ensuring that the entire beam surface was covered. This was left for a period of three hours before adding 2.5 mL of medium to the plate and then, incubated overnight at 37° C. with 5% CO2.

### Test Protocol

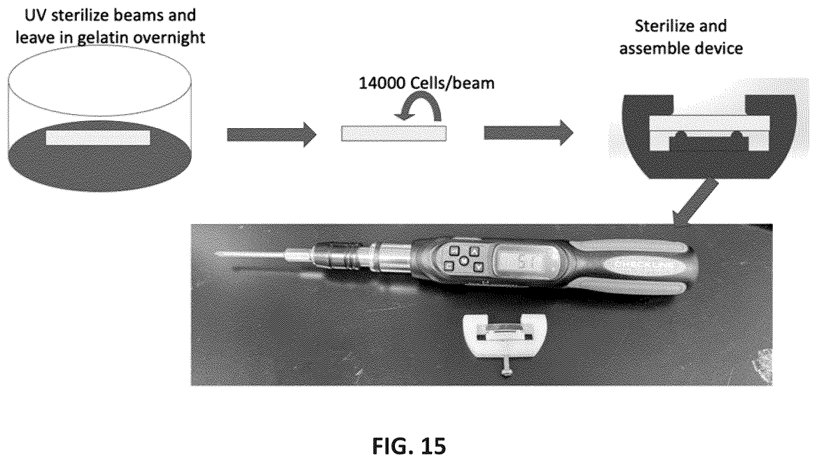

The device was prepared as per the testing protocol detailed above, but this time using the beams with cultured cells on top. The cells in the midsegment of the beam (along the score mark) were then observed via an EVOS fluorescence microscope. Cells were stressed during 30 min under the microscope in order to generate a real time stress time-lapse. In addition, five to six images along the length of the score mark were taken following the time-lapse. FIG. 5 provides a visual representation of the methods used to prepare the beams and preform the tests. FIG. 15 shows a graphical representation of the test protocol followed during this study.

### Image Analysis

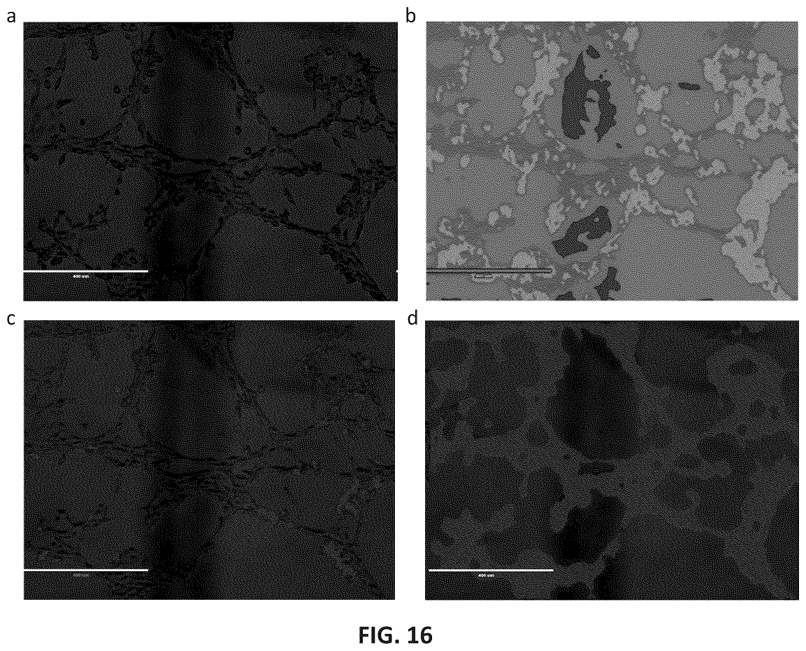

MATLAB® was used to try and develop a cell tracking algorithm. Two different methods were attempted, one to use bright-field images and another method to use a nuclear stain. In both cases the cells were segmented from the background then the cells were correlated to the cells in the next image. Finally, the movement from one frame to the next was calculated. FIG. 16 shows the one of the methods used to segment the cells. FIG. 6. A live and dead stain of the bottom (compression), middle (neutral) and top (tension) of the beam showing that the stress is not affecting cell viability. Panel a of FIG. 16 shows the test image, panel b shows the k-means clustered image with 2 groups encompassing the cells, panel c shows an intensity based approach to segmenting the cells, and panel d shows the final segmented image. Panel a of FIG. 15 shows an unprocessed image taken on an evos fluorescence microscope, panel b shows the k-means clustering of the row image, panel c shows the edge detection method of segmenting the cells, and panel d shows the final segmented image highlighting the detected cell area in blue.

### Phalloidin Staining

After stressing the cells for 30 min, the beams with the cells were removed from the device and washed with PBS 3 times. The cells were then fixed using a 4% paraformaldehyde solution for 15 minutes. The fixing solution was aspirated, and the cells were left in 1% triton x in PBS for a total of one hour and 30 minutes to permeabilize the cell membranes. The triton x solution was removed and the cells were left in a 2% BSA solution for 45 minutes to block unspecific proteins. After 45 minutes, the BSA the solution was removed, and the cells were washed with 1% triton x 3 times and a 1:1000 solution of phalloidin in 2% BSA was then added and left on the cells for one hour. After the hour, the phalloidin solution was removed and a 1:8000 solution of DAPI was added for 3 minutes. After the 3 minutes, the cells were washed with PBS 3 times and 2 ml of PBS were then added to the samples. The cells were stored at 4° C. for imaging the following day.

### Cell Viability

Cell viability was tested using a LIVE/DEAD viability kit from Invitrogen®. This kit comes with 2 parts, one part being the staining for the live cells (green) and one being the staining for the dead cells (red). The live component was added to the dead component and mixed thoroughly by repeated pipetting. The mixture was then diluted in a 1:1 ratio with grow medium and add it to the samples (beams with cultured cells on top). Cells were incubated in the live/dead solution for 20 minutes. The cells were then imaged on an Evos® fluorescence microscope using a green (488 nm) and red filters (555 nm). This was followed by stressing the cells as per testing protocol.

### Actin Visualization

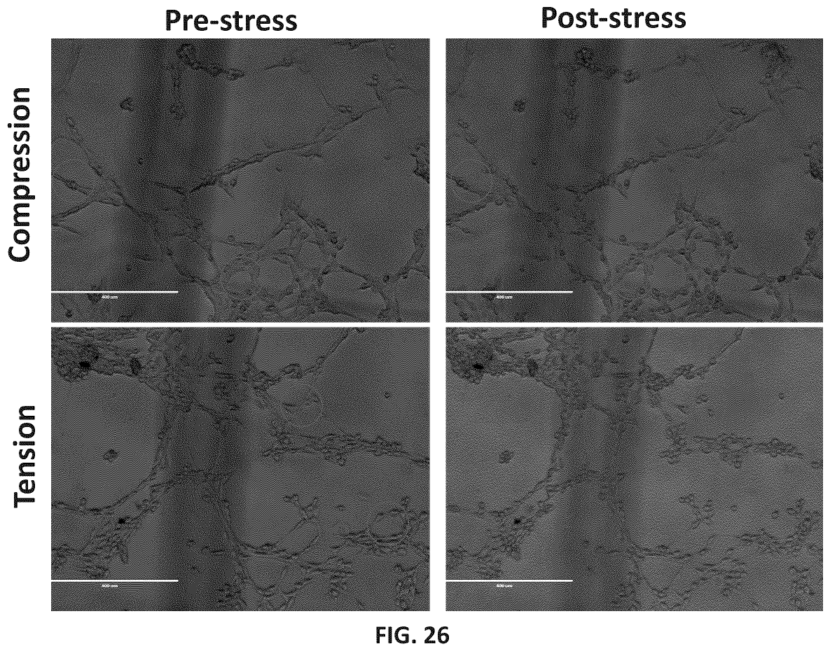

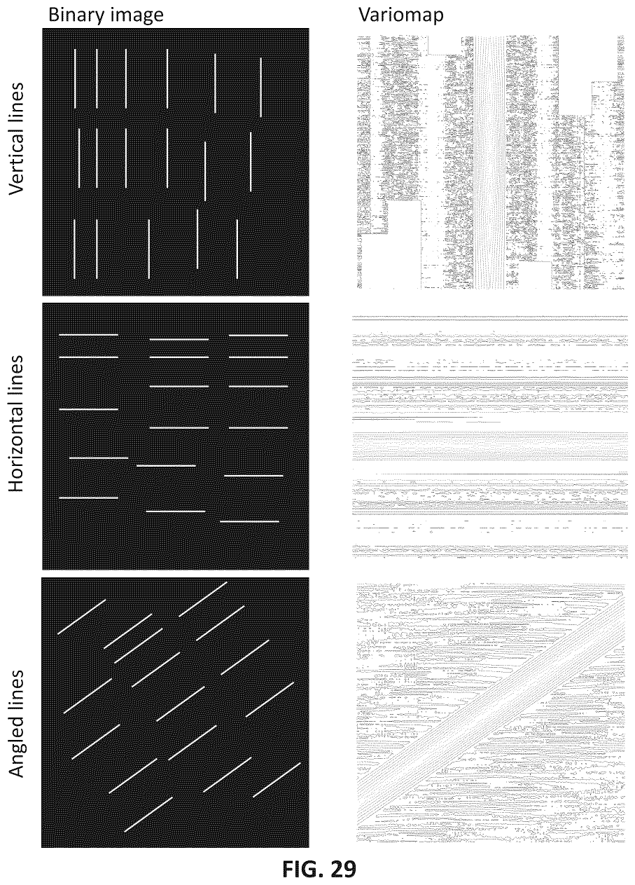

A MATLAB® algorithm was used to visualize the effect of stresses on the actin filaments, which were stained with phalloidin. The first step in the algorithm was to isolate the actin cytoskeleton from the rest of the image and remove some of the noise. This was done by first isolating the red color channel from the rest of the image, applying a grey threshold using the MATLAB® built-in function ‘greythresh’, and then segmenting according to the method of Niblack [62, 63]. It was assumed that the actin filaments would have a higher mean than neighboring pixels. Thus, a mean and standard deviation of the pixels within 9 pixels of any given pixel was calculated. Any pixels that satisfied both the grey-threshold filter and had a value at least half a standard deviation above the mean of the 9-pixel radius area were kept. The resulting image was then run through a 2-D autocorrelation algorithm. The centermost section of the resulting variomap was kept. Discuss of test images and the resulting variomap are shown in FIGS. 26 and 29, as discussed in Appendix C.

### Statistical Analysis

All analyses were done using MATLAB and results were presented as means±standard deviation. Student t-tests were used for comparison between 2 groups and ANOVA was used for comparisons between multiple groups. Tukey's honestly significant difference procedure was used to determine significance between multiple groups. A p-value of less than 0.05 was considered significant.

### Results and Discussion for Aim 2

### Cell Survival and Material Biocompatibility Assay

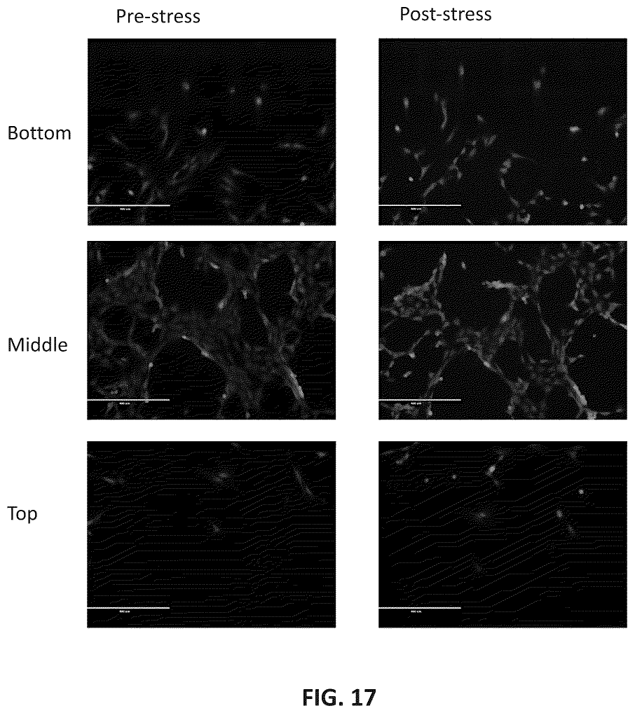

To assess the cell survival of the cell under stress and device biocompatibility, a Live/Dead staining was performed. The cells were stained as described above and imaged before and after the stress. FIG. 17 shows a representative set of images of the top bottom and middle of the beam representing the cells in tension, compression, and neutral respectively. In addition, control samples were tested to ensure that the cell stressor did not contain any molecules that were getting leached of and causing cell death. There were no visible differences regarding cell viability in any of the segments of images. This showed that the type and magnitude of the stresses being applied to cells were not affecting cell viability. Additionally, it showed that materials used to fabricate the cell stressor can be used in the cell medium environment without being toxic.

### Image Analysis Algorithm Development

Two different algorithms were developed for tracking the cells-one that tracked only cell nuclei and one that was meant to track the entire cell. The attempt at tracking the whole cell used brightfield images taken on the EVOS microscope. While the nuclei tracking relied on the use of a nuclear stain and the DAPI fluorescence channel on the EVOS microscope.

FIG. 7 shows the test image used along with the five images for the different methods of edge detection. The sobel, prewitt and roberts methods were underestimating where the cells were located. The Canny and log methods were both overestimating where the cells were located with the log method showing less overestimation than the canny method.

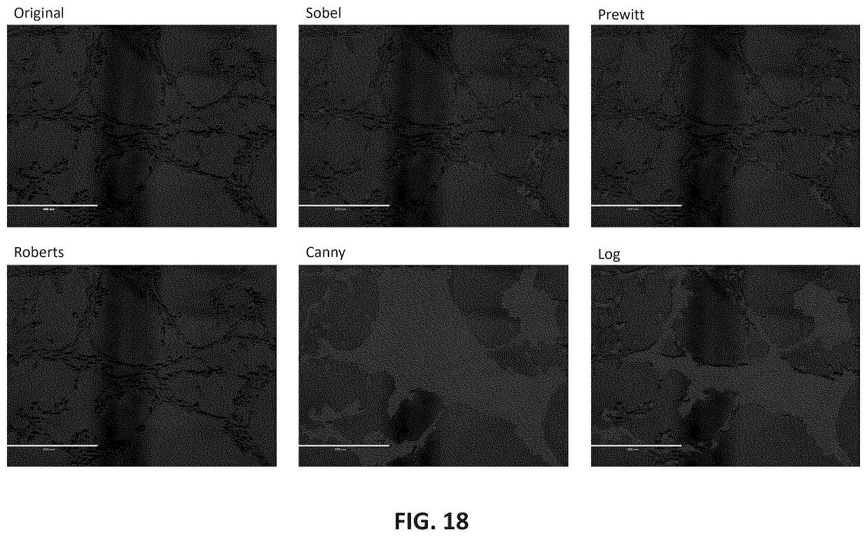

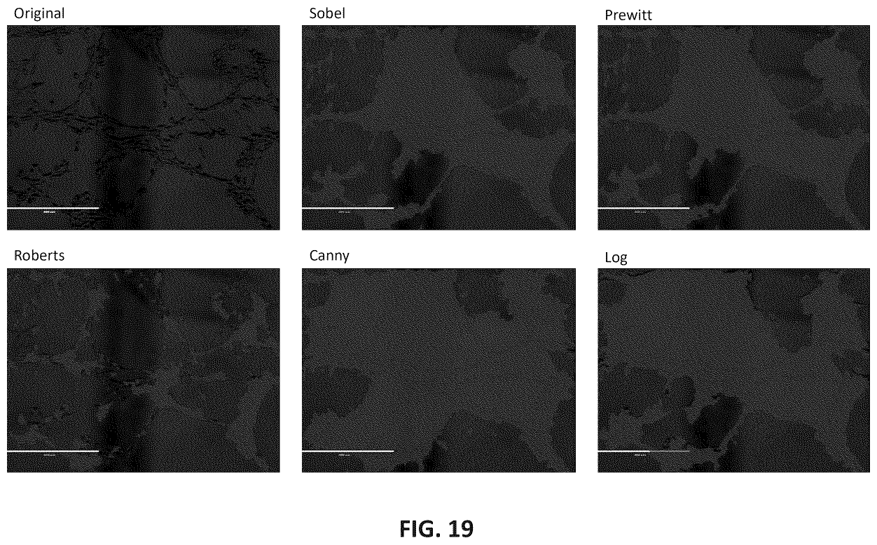

The first attempt at segmenting the cells from the background relied on changes in pixel intensity combined with an edge detection function. Five different edge detection algorithms were attempted using the MATLAB edge function. The five algorithms were sobel, prewitt, Roberts, canny, and log (FIG. 18). From this group of algorithms, the log method seemed like the most promising method to use for this image as it was able to detect most of the cells and was not greatly overestimating the cells like the canny method. One way to potentially increase the detection of the algorithms is to lower the threshold being used. This was done by running the image through the edge detection function once, capturing the threshold that would have been used, then halfling that value and using it as the threshold for the edge detection function (FIG. 19).

FIG. 8 shows the test image used along with the five images for the different methods of edge detection using half of the threshold automatically identified. The sobel, prewitt, canny, and log methods were all severely overestimating where the cells were. The Roberts method was only slightly underestimating the area covered by the cells.

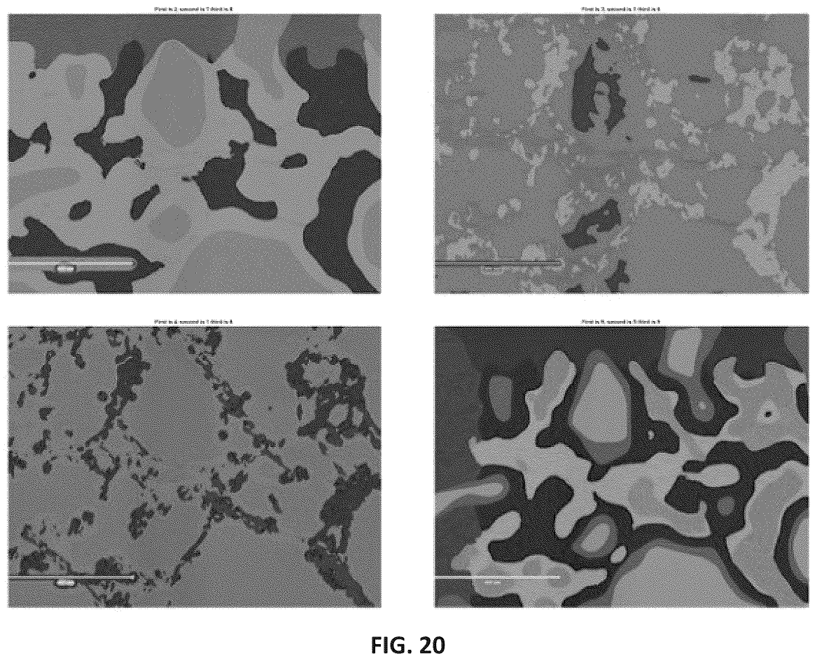

From this set the Roberts method showed the most promise. The prewit and sobel methods showed the same overestimation observed in the first set canny method. Both the canny and log methods were greatly overestimating the area where the cells were. From both sets the one that seemed the most promising was the Roberts method with half the threshold it would have used. However, when finetuned to the one test image, the algorithm would have difficulty detecting cells in different images that had slightly different lighting. This was especially true when the cells where in the shadow area where the etch indicating the middle of the beam was. To try and overcome this limitation, a texture-based approach was implemented. A garbor filter was chosen for the texture-based approach. The garbor filter was combined with k-means clustering for images to segment the image. This approach was largely based on the MATLAB® example code for texture-based segmentation. There were a few parameters in the code that could be changed to have better detection (FIG. 20). For example, the number of groups for the k-means clustering helped improve the detection. Using nested for loops to vary the parameters, a PDF file was generated. From the PDF the combination that had the best detection was chosen. This combination had two groups capturing the cells. To identify which two group captured the cell, the previously described segmentation using intensity was used. The groups that identified similar area to those identified by intensity segmentation were selected. This method of combining a garbor filter with k-means and intensity-based segmentation seemed to work on most images.

FIG. 9 depicts examples of how the different parameters used for the texture based approach identified the cells. These representative images show how the kmean algorithm varies as the parameters were changed; while there were many parameters that correctly identified the cells (bottom left and top right in FIG. 19), the one that was chosen was the one with the lowest parameters as that took less processing time.

The first step in tracking the nuclear stain was isolating the nuclei from the background of the image. This was done by using the MATLAB® image binarization function. This function sets a threshold based on pixel brightness. Any pixel that is lower than the threshold value is set to 0 and everything over the value is set to 1. Fortunately, on fluorescence microscope images, the background is distinct from the stain and no further steps are necessary to separate the background and foreground. The next step in the algorithm was detecting the nuclei and recording the centroid of each one. This was done using the MATLAB® regionprop function which reliably identified the nuclei. The third step was correlating each nuclei in one image with the same nuclei in the next image. For this, the assumption that the nuclei were not moving significantly was made. This can be justified because the average migration speed of 3T3 cells was seen to be 0.48 micrometers/minute [37], which correlates to about 0.51 pixels/minute in the image. This step was accomplished by using the MATLAB distance function which finds the distance between a set of points and the closest point. This function was given the set of centroids from 2 consecutive images which returned both the distance between the closest points as well as the index of the set of second points that correlated to the first set of points. The centroids of all the nuclei were then reordered to match the indexing of the first image of the time-lapse. The final movement after 30 minutes of stress on the beam was calculated using the first and last image of the time-lapse. The distance traveled was calculated using the reordered list of nuclei and the MATLAB distance function. The angle of movement was calculated by assuming that the first image was the origin and using an inverse cosine function.

### Migration Analysis

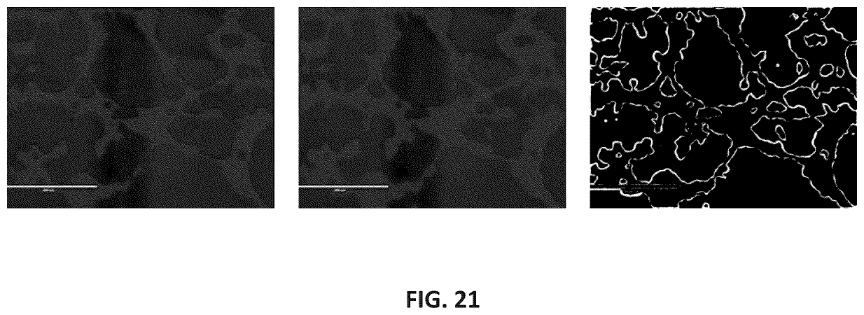

As previously mentioned, assessing the cell migration proved difficult. The original algorithm that outlined the entire cell body was not precise enough to use as the cells moved on the scale of pixels so any minor inaccuracies in the algorithm lead to loss of significance. FIG. 21 shows the same image run through the algorithm twice. While it seems like the same spots are highlighted, there was a small error that can be seen by subtracting the 2 masks (right panel). FIG. 21 provides the same image ran through the algorithm twice, where the right most image is the masks subtracted.

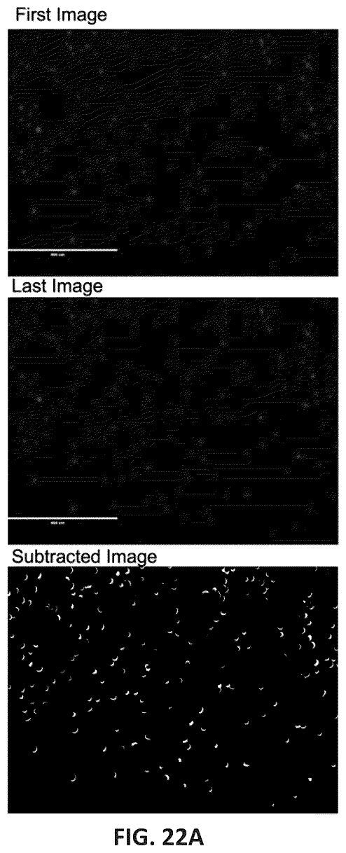

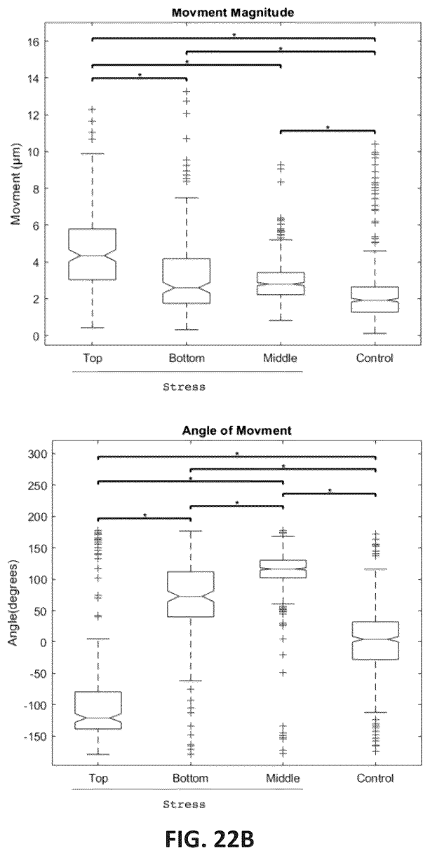

This led to the use of a nuclear stain in order to quantify cell movement. The downside of using the nuclear stain is that it was not apparent that there were any differences between the first and last frame of the time lapse (FIG. 22A). To view the minor differences, the images can be subtracted (FIG. 22A, subtracted images). This show the areas where the nuclei position was not previously but are currently located. While it was seen that in all samples, there were nuclei that pointed in a random direction, there is a significant portion of nuclei that showed directional alignment.

FIGS. 22A and 22B illustrate the migration analysis using a nuclear stain. In FIG. 22A, the first and last image of the time lapse preformed using a nuclear stain. The subtracted binary image shows the differences between the “first image” and the “last image”. FIG. 22B provides quantification of the nuclei migration/alignment showing significant differences in nuclei movement/alignment towards between the different groups. In the stress group top is tension, bottom is compression, and middle is neutral. p>0.0001.

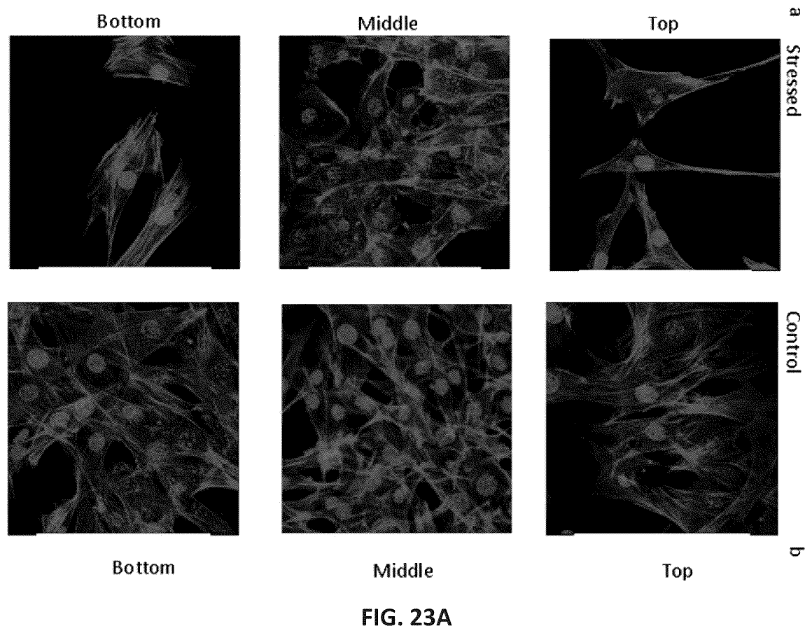

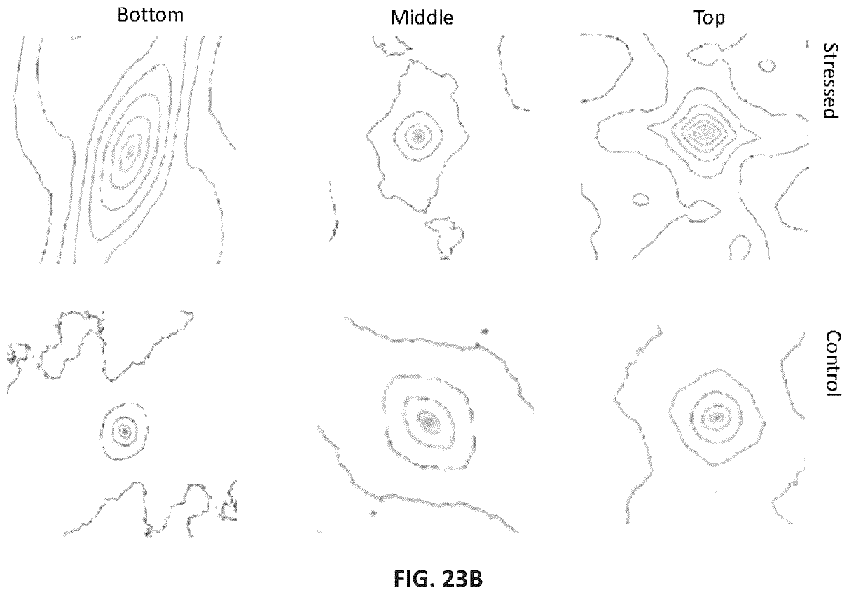

Using this algorithm, the nuclei pointed/aligned the most when the nuclei was under tension (FIGS. 23A and 23B). Interestingly the nuclei were pointing toward the area of higher tension as can be seen by the negative angle of movement which means that the nuclei were aligning toward the bottom of the image which is the side under higher tension. This was the same with the nuclei experiencing compression. This can be seen as the positive angle meaning the nuclei were pointing toward the top of the image. The segment with the nuclei in the neutral segment of the beam also saw significant alignment toward the side with compression. This could have been due to several factors. The cells could have had a better “connection” to the side with the cells in compression and that signal affected them more than the cells on the tension side. It should be noted that the cells under tension were at lower confluency than the side in compression. These results correspond with the results observed with the cytoskeleton alignment which is discussed next.

### Structural Analysis

2-D autoclustrogram analysis, to visualize the actin staining, showed some interesting results. It was observed that in both cells under tension and compression, there is a clear major axis. This means that the actin filaments were aligning inside the cells (FIG. 23B). In the middle (neutral) segment of the beam, there was not a clear well-defined major axis.

In the control sample, there was no clearly defined axis. This can be seen with the center of the autoclustrogram being circular. This shows, as was seen in other studies, that stress lines up the actin fibers within a cell [52].

FIGS. 10A and 23B show phalloidin staining along with the visualization of filament orientation using 2-D autoclustrograms. In the stressed samples (top and bottom), there is a clear major axis in the center of the variomap indicating that the actin fibers are aligning. In the stressed middle there is no clear major axis but there are a couple starting to occur. In all the control samples there is no major axis indicating that the is no alignment of the actin fibers.

### Conclusion

The long-term goal of this project is to engineer a cells stressor that mimics the stresses that happen in the heart and its environment under both pathological and physiological conditions. With this aim, it was necessary to first work on the design of a functional cells stressor and to show that cells can in fact detect and react to the magnitude and type of stress being applied by this device. Therefore, the objective of this study was to engineer cell stressor devices, systems and methods that can apply both compressive and tensile stresses simultaneously on cells. Additionally, it was important to see that cells were able to detect and react to the type/amount of stress that is being applied. This was measured through the use of cell tracking and basic analysis of the cytoskeleton using an actin stain.

The cell stressor was based on the prototype previously made by our collaborators, which was not functional to assess mechanical stresses on cells. In this investigation, we improved the first prototype of the cells stressor to make it functional, and able to perform repeated experiments without difficulty in culturing cells and transporting the device. The applied strain on the beams was found to be 0.01 which agreed with the analytical calculations. This meant that the torque screwdriver was indeed a reliable method of applying the force unto the beam. This method also minimized the strain applied in comparison to other studies. While this study had strain values of 0.01%, most other studies used strain between 1-20% [52, 64-68]. This means that the cell stressor is able to apply large stresses while maintaining minimal strain, so the effects of just stress can be better analyzed.

Preliminary studies at how cells would react to the stress were performed using 3T3 cells. A live/dead stain was done for 30 minutes to ensure that the stress applied was not enough to cause the cells to undergo apoptosis. There were no differences in the staining between the pre- and post-stress samples indicating that the cells were surviving the stress test. However, a limitation of this test is that detached cells were not accounted for.

The 3T3 cells' nuclei's alignment preferences were also studied. It was found that the nuclei preferred pointing towards the area of higher stress whether that be compressive or tensile. This was assessed using a nuclear stain and a cell tracking algorithm developed in MATLAB®.

The staining for actin revealed that the actin cytoskeleton of the cells was able to align or start aligning, which correlates with the nuclei alignment results. In previous studies it was shown that cyclic strain was able to align the cytoskeleton [52] of cells, which was seen in this study.

In conclusion, we engineered, fabricated, and tested a cell stressor device that can apply both tensile and compressive stresses simultaneously to cells. The tests performed showed that the stress applied was not causing apoptosis, and the cytoskeletons and nuclei of the cells were aligning. This device can be used not only for 3T3 cells, but for other organ cells that are exposed to stresses, such as eye cells, heart cells, lung cells, etc.

### Future Work

### Beam Improvements

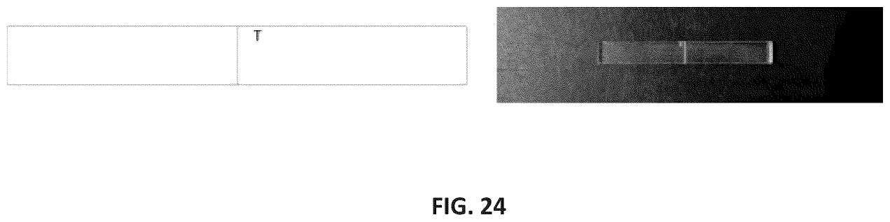

While the cell stressor device according to the present technology, and as described in this Example 1, was sufficient for the uses mentioned above, there are several factors that may be the subject of further improvement. One of the improvements that can be done is having a better beam design. During this study, the middle of the beam was scored with a razor by hand. While this was usually consistent, a way to improve this would be to laser etch the center mark. This would ensure that the center of the beam is always consistently marked. In addition to this, a way of marking which side of the beam was the side in tension would be ideal to ensure that even if the beams are rotated during the staining process, the side that was in tension can be distinguished. These improvements were done and shown in FIG. 24, but the improved design was not used for this study, as discussed in this Example 1. Further improvements to the beam design would be to etch a grid to the back of the beam that would make tracking cells over a longer period easier by providing more points to align the images. This would however need the laser cutter parameters to be finetuned further to ensure that the beam is not fully cut through and that the etches are not overlapping.

FIG. 11 depicts the improved beam design for the cell stressor device. On the left is the ADOBE Illustrator design that was replicated to fill an entire PPMA sheet, and on the right is an example of a laser cut improved beam design.

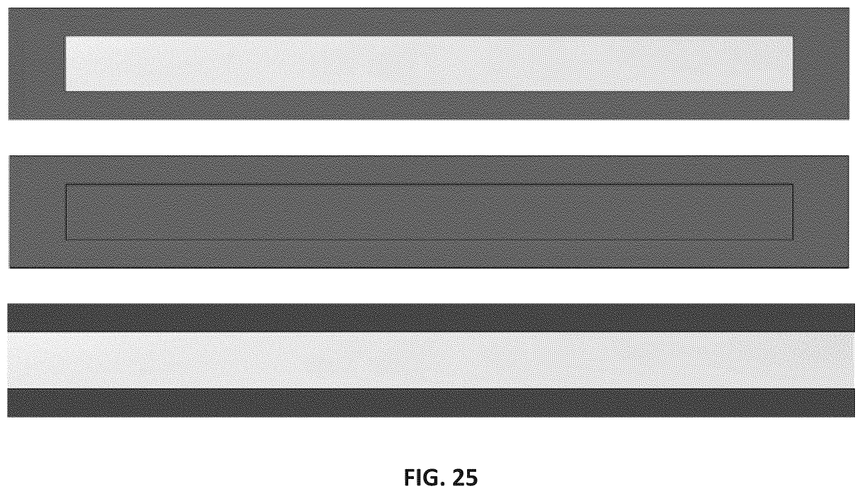

Another improvement that can be made to the beam design is the addition of a well to allow for the use of gels with the cell stressor according to the present technology. This can be done in a few different ways: 1) a complete removal of a box segment in the middle, 2) a partial removal of a segment in the middle of the beam, and 3) the complete removal of the middle of the beam. FIG. 25 shows a SOLIDWORKS® render of the three different designs.

FIG. 12 depicts beam renders for use with gels. The top panel show a SOLIDWORKS render of a beam with the middle rectangle completely removed. the middle panel show a render of just the top 1 mm removed creating a well. The bottom panel shows a render of the middle segment completely removed.

### Bender Improvements

Another improvement on the design would be to add more support for bender according to the present technology, as discussed in this Example 1. Currently, the bender is held in place by relying on the screw and the beam to keep it in place. During assembly of the cell stressor device, it was sometimes difficult to get the bender to stay in place while the beam was inserted. This can be improved upon by adding more space for the screw to sit which should help keep the bender in place. Additionally, 2 pins can be added to the bender that align with holes in the main body. This would make the bender easier to align and help hold it in place when adding the beam.

### Confluency Experiments

The results seen and discussed in this Example 1 were promising and will help guide future studies. As seen in several studies, the confluency of the cells affects mechanotransduction to neighboring cells [69, 70]. Since this study was only focused on one cell confluency, it could be possible that different confluences might have different reactions in the middle of the beam where there is minimal stress and the cells should be able to choose which side they migrate to.

### Increase Study Duration

During this study only a 30 minute time-lapse was taken. While it was seen that the cells preferred migrating towards higher stress during that time period, it is still unknown if that persists. Extending the time-lapse duration would allow for a more complete understanding of how the cells react in the long term.

### Cell Co-Cultures

One question that remained unanswered is how the stress affects a co-culture of cells. In one study it was shown that fibroblasts were coupled to cardiomyocytes and changes in the fibroblasts affected the electrical properties of the cardiomyocytes [71, 72]. During this study, it was seen that the fibroblast changed mechanically. So, if they were cultured with cardiomyocytes what changes to the cardiomyocytes would have occurred? Another thing that was not analyzed during this study was the ECM secretion from the fibroblasts. It was shown that upon activation, myofibroblasts have increased ECM synthesis.

### Algorithm Improvement

While it was seen that the 3T3 cells showed a preference towards migrating to areas of higher stress using the nuclear tracking algorithm some information was lost. As seen in the bright field time-lapses there was movement toward and away from other cells (FIG. 26). This information was lost when using the nuclear stain. Further developing and refining the algorithm would make it possible to view these changes as the cell migrates. A potential method to achieve this would be to train a neural network to identify the cells.

FIG. 13 depicts a brightfield image of the cells pre and post stress showing some movement.

### Additional Devices

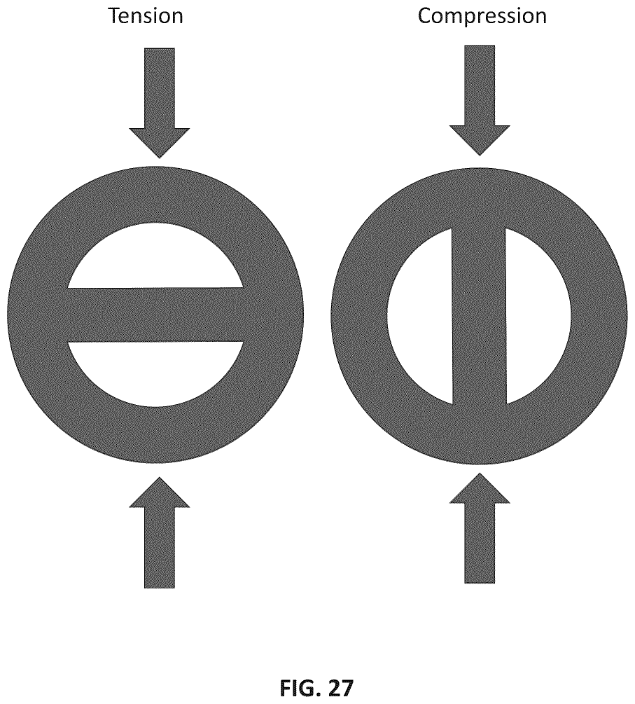

During this study, it was seen that cells did not have a clear preference at avoiding a certain type of stress. The next step would be seeing if there were any differences in gene expression in cells under either type of stress. Using an omega-ring design (FIG. 27) it is possible to achieve pure compressive and tensile stresses simply by rotating the ring.

FIG. 14 depicts the aforementioned omega ring concept. This design can have either tension or compression by rotating the shape.

### Understanding Mechanical Stresses: A Cell Stressor Device for In Vitro Modeling of Cardiac Biomechanics in Health and Disease

Mechanical stress is a major driver of cardiac hypertrophy and left ventricular remodeling post-myocardial infarction and can trigger pathological scarring, or fibrosis which can lead towards the progression of heart failure (HF). HF is an expanding health problem affecting >6.2M Americans and costing >$30B annual.

Although it is well known that cells respond to changes in their mechanical environment, the molecular mechanisms underlying this ability, the mechanoreceptors which receive mechanical stress and convert it into intracellular biochemical signals, and how cells interact with each other under pathological and physiological stress conditions are still poorly understood. This is in part attributed to the lack of proper in vitro systems that can recapitulate cardiac forces and injury, such as tension and compressional stresses that occur during cardiac damage and recovery. The disclosed cell stressor devices, systems and methods according to the present technology enables study of the effects of mechanical stresses on cells: a cell stressor which applies both tension and compressional stresses simultaneously to cells.

The device (e.g., cell stressor device 10) was designed based on a four-point bend test as outlined by the standard ASTM E855 08. The device is composed of five parts: 1) the main body, which may be designed to fit in a 60 mm culture dish and provides the two outer supports of the 4-point bend; 2) the bender, which is the segment that receives the force and had the two loading point (e.g., the aforementioned pair of nubs) for the 4-point bend; 3) and means for moving the bender to apply the force to a beam and, 5) the beam, which is where cells are cultured and which is the one being subjected to mechanical stresses.

At least a portion of the cell stressor device according to the present technology can be three dimensional (3D) printed using Acrylonitrile Butadiene Styrene (ABS). The bolt and nut may be both stainless steel and the beam is made of polymethyl methacrylate (PMMA). However other materials can also be used. Cells can be cultured in monolayer, on top of the beam, or in 3D using a modified beam for adding decellularized tissue in the middle. Mechanical stresses can be applied manually or motorized. The purpose of our device is to model the mechanical stresses that occur in cardiac tissue during pathological and physiological conditions. Use of the cell stressor device according to the present technology may be utilized to elucidate the mechanisms by which mechanical stresses activate mechanotransduction signaling pathways that cause fibrosis and pathological hypertrophy in the heart. The results of such utilization may lead to novel therapeutics for cardiac injury and for other tissues that undergo mechanical stresses and that also exhibit fibrosis (lungs, kidney, etc.). Additionally, since fibrotic diseases account for 1/3 of deaths globally, the devices, systems and methods according to the present technology could be applied to elucidate the origin of fibrosis in various organs instead of or in addition to the heart.

The cell stressor device and associated system and method allow for 3D cell culture assessment using natural and synthetic hydrogels. Both tension and compression can be evaluated at the same time which mimics better the stress that happen in the native tissue. The embodiments of the present technology are uncomplicated and inexpensive to produce since they use very simple materials. dECM and other proteins can be incorporated to better mimic the tissue environment. The cell stressor device according to the present technology can be used to do live imaging analysis. The cell stressor device according to the present technology can minimize strain while applying stress.

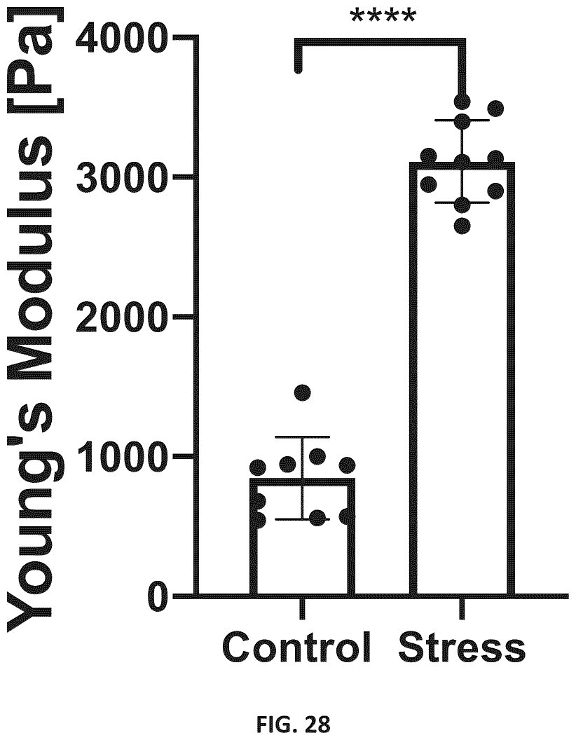

FIGS. 28 and 29 are discussed in the Appendices.

### Example 2

### Understanding Mechanical Stresses in Women and Men: In Vitro Modeling of Cardiac Fibroblast Biomechanics

Heart failure (HF) is an expanding health problem affecting >6.2M Americans and costing >$30B annually. While there are therapies aimed at slowing HF, final stage HF requires a heart transplant and there is no cure. Cellular mechanical stress is a major driver of cardiac hypertrophy which can lead to HF. Additionally, the heart varies in several ways between women and men and while HF is prevalent in male and females equally, the diagnosis standard and treatments were developed based on the male heart. As a result, the standards of care and therapies cause women to receive a delayed diagnosis thus decreasing efficacy of treatment during heart failure. Fibroblasts in the heart are responsible for deposition of extracellular matrix (ECM) as well fibrosis in response to stress or injury. Therefore, it is crucial to understand how stresses affect cardiac fibroblast cells of their respective sexes. This study utilizes a novel cell stressor device that allows compressive and tensile stresses to be exerted on cells. Male and female cardiac fibroblasts were exposed to mechanical stresses using the cell stressor. Changes in cytoskeleton, fibroblast activation, and focal adhesion were assessed using immunofluorescence staining. As controls, we used fibroblasts with no stresses applied in order to compare the stress with no stress through immunofluorescence staining and its respective quantifications. To understand the mechanobiology behind these differences, ion channels and mitochondria function were also investigated by staining. Differences were observed in cytoskeleton organization, fibroblast activation, focal adhesion and changes in Piezo1 ion channel and mitochondria in males and females.

### Introduction

### Defining the Heart

The heart is a contractile mechanical organ that beats >2.5 billion times in a lifetime [73] and experiences large pressure stresses and tissue strain through every systolic-diastolic cycle [74]. To adapt cardiac output to hemodynamic conditions, the heart undergoes intrinsic mechanisms [75] at the cellular, tissue and organ level to generate and respond to mechanical stress [76] and regulate cardiac function under both physiological and pathological conditions. Furthermore, the heart also adapts to the stimuli by lengthening the cardiomyocytes (dilated cardiomyopathy) [77]. Mechanical stress is known to trigger hemodynamic over-load which ultimately causes cardiac hypertrophy [78] (cardiac enlargement) and left ventricular (LV) remodeling post-myocardial infarction (MI), regulating tissue fibrosis [75], and this pathological cardiac remodeling can lead to the progression of heart failure (HF) [78].

### Sex Differences in the Healthy Heart

Female and male hearts vary in several aspects. The weight and size of the heart differ throughout the lifespan and are dissimilar between women and women [79]. Variations in size of the heart between males and females can be noted once puberty begins [79]. These differences include heart rate where the average adult male heart beats 70 to 72 times per minute while the average adult female heart beats 78 to 80 times per minute [79]. This can be primarily attributed to the size of the hearts where the male heart is larger than the female heart, thus able to pump more blood with each beat [79]. Contrarily, the smaller female heart requires more beats to pump blood to match the male cardiac output [79]. Females have a contrasting intrinsic rhythmicity to the pacemaker of their hearts which results in a faster heartbeat [79]. During infancy and the early stages of life, LV mass is similar in females and males [80].

Following puberty, male heart mass increases between 15-30% depending on body size proportion [79]. Consequently, male myocytes exhibit greater hypertrophy than its female counterpart [79]. In females, the myocyte quantity and size are maintained during aging while males lose around 64 million cardiac myocytes per day [79]. The male loss is due to compensatory hypertrophy (increase of cellular volume [80]) to regulate sufficient heart mass [79]. The female heart exhibits greater diastolic function compared to the male heart, though both sexes demonstrate reduced diastolic function as aging progresses [79]. A decreased systolic function in men than women is also observed [79]. A particular difference is noted in the left ventricular size and function [81]. The LV elastance/stiffness is higher for women in both the systolic and diastolic [81].

Both sexes experience increased septal and wall thickness as age advances [79]. Left ventricular diameter increases in males whereas it does not in females, indicating that myocardial mass is lost in males and not females [79]. Consequently, males display 25-38% higher LV mass [80]. As a person ages, the disparity in LV stiffness grows, as it increases more in females than males [81].

### Pharmacological Sex Differences

To understand sex differences in the outcomes of HF, it is significant to explore pharmacology and devices and their roles in contributing to the medical inequity of women. Studies have demonstrated that the maximum plasma concentrations of many angiotensin-converting enzyme inhibitors (ACEI), angiotensin receptor blockers (ARBs), and beta-blockers can be 2.5 times higher in women than men in spite of similar dose administration [82]. Pharmacokinetic sex differences that may lead to these higher plasma concentrations in women are linked to principal variations in body composition (women having much lower weight and height, a larger proportion of body fat, and a decreased peripheral distribution volume) [82]. The lower body composition leads to reduced glomerular and hepatic filtration rate, slowing down drug clearance and elevating further the plasma concentrations [82]. Some cytochrome P450 isoenzymes appear to have less expression/activity in women, thus affecting drug metabolism for many HF treatments [82].

### Epidemiology of Heart Failure

Heart failure is an expanding health problem affecting >6.2M Americans and costing >$30B annually [83]. It is an expensive and morbid cause of hospitalization, carrying a lifetime risk of 20-45% for adults, is the leading discharge diagnosis among the elderly, and incurs costs of ˜$25K per patient per year [84,85]. According to the National Health and Nutrition Examination Survey [86], the number of deaths due to HF was around 80,480 in 2017 (36,824 males and 43,656 females) and the prevalence of HF among US adults from 2013 to 2016 was: 0.2 and 0.3% in the age range of 20-39 years old in females and males respectively, 1.7 and 1.2% in the age range of 40-59 years old in females and males respectively, 4.8 and 6.9% in the age range of 60-79 years old in females and males respectively, 12 and 12.8% above the age of 80 years old in females and males respectively [87]. In addition, data from large registries suggest sex-related differences in transplant-free survival patients with HF [82, 88-90]. However, the processes by which sex-related variations in specific biological pathways alter the progression towards HF in men and women need further investigation [91,92]. Specifically, sex-related differences that affect biological mechanisms driving the biomechanical phenotype of fibrosis in female patients needs further study.

### Sex-Specific Differences in Heart Failure

Cellular mechanical stress is a major driver of cardiac hypertrophy [78] and left ventricular remodeling post-myocardial infarction [75] and can trigger pathological fibrosis which can lead to the progression of HF [93]. Men are susceptible to HF with reduced ejection fraction (HFrEF) while women are susceptible to heart failure with preserved ejection fraction (HFpEF) [82]. This means men present eccentric hypertrophy (volume overload [94]), stubborn inflammation, and fibrosis and apoptosis [82]. Women present with concentric hypertrophy (pressure overload [94]), minimal inflammation, fibrosis and apoptosis [82]. Data suggests that HF pathogenesis differs between sexes [95-97]. For example, hearts in females have smaller and stiffer left-ventricles and higher ejection fraction (% of blood that the left ventricle pumps out with each contraction) than males [95]. This higher stiffness of female hearts has related to an increase in cardiac fibrosis at old age. In younger women, estrogen reduces collagen production in female cardiac fibroblasts, but stimulates it in males [95]. Cardiomyocytes are more abundant in the female myocardium than the male myocardium [77]. Furthermore, the female myocardium has smaller, less elongated ventricles than the male and was also more texturally intricate [77]. In addition, lipid and energy metabolism is better maintained in female stressed hearts than in males [95]. A significant determinant of HF outcomes in women that does not apply to men is pulse pressure [95].

Women with HF survive longer than men and have a lower risk of sudden death [95], however women are frequently overlooked by routine exams and diagnosed with HF later and with more severe symptoms than men [77]; moreover, women are less adequately treated since most therapeutics are derived from predominantly male studies [96]. Particular cardiomyopathies such as Takotsubo and peripartum are rare and affect only women and there is an understanding that women need lower doses of beta-blockers and angiotensin-converting enzyme inhibitors than men would [95]. However, there are very few studies that separate the effects of treatments for men and women [96]. Clinically, HF presents differently between men and women where men show coronary artery disease (CAD) and myocardial infarction (MI) while post-menopausal women exhibit hypertension [96]. Men develop abnormal forms of hypertrophy whereas for women it is more concentric [96]. Studies have shown that while pathological mechanical stress is a major driver of fibrosis and can promote progression of HF [96], there is limited understanding of the biological mechanisms and effects of sex-differences on cardiac biomechanics, particularly how this affects women's health.

Particular cardiovascular conditions within HF also display sex differences. For example, hypertension is more commonly observed in female HF patients than male, 50% and 40%, respectively [81]. Hypertension raises the chance of HF in females by 3-fold and in males by 2-fold [81]. The reason for this is thought to be due to a greater augmentation index (arterial stiffness indicator) between central and peripheral blood pressure in females versus males [81]. That leads to increased damage like LV hypertrophy [81]. Additionally, atrial fibrillation seems to heighten the risk of HF in women but not in men as well as the chance of hospitalization [81].

Extracardiac sex differences also contribute to cardiac function [81]. In regard to the immune system, females display more aggressive immune responses than males which leads to quicker and more efficient recovery from diseases as well as vaccine efficacy and cancer survival [81]. With this stronger immune response also comes with particular drawbacks for females [81]. For example, they have increased proinflammatory gene expression, higher CD4 and CD8 T-cell activation, greater inflammatory cytokine levels, and a generally increased systemic inflammation [81]. This heightened inflammation contributes to vascular dysfunction [81].

### Sex Differences in Hypertrophy

Pathological hypertrophy is a common precursor for HF [94]. Females tend to show a more favorable state of hypertrophy than males [94]. Cardiac hypertrophy is a key risk factor for heart failure in females [94]. However, pre-menopausal women are better guarded against cardiac hypertrophy, but this disappears after menopause. This is due to women having higher levels of estrogen in their pre-menopausal stage than men and thus counteracting pro-hypertrophy signalizing [94]. Androgen, on the other hand, which is higher in men, has the opposite effect [94]. Women who receive hormone (conjugated equine estrogen) replacement therapy closer to menopause tend to have a decreased risk of cardiovascular disease in comparison to the increased risk among women more distant from menopause [94]. In patients with severe aortic stenosis, the ventricular remodeling is more severe in men leading to a poorer prognosis than that of women due to a higher inflammatory response and fibrosis [94]. A clinical study showed women had a longer 5-year survival rate than men [94]. Following valve replacement, the left ventricular ejection fraction in females is much higher than in men, indicating a more favorable adaptation to long term mechanical stress loads in women than their male counterparts [94].

### Sex Differences in Risk Factors

Diabetes has been shown to be more significant in the context of HF in women [82]. A study showed that diabetes was a greater risk contributor of HF in women (five-fold) than men (two-fold) [82]. Women with diabetes had higher indications of adverse LV remodeling along with increased LV wall thickness, relative wall thickness, and LV mass index [82]. Obesity is strong risk factor for developing HFpEF than HFrEF and also to a more noticeable extent in women than men [82]. Worldwide, obesity is higher in women, who are also more likely to develop HFpEF in comparison to HFrEF if obese [82].

Pregnancy being unique to females is a cardiovascular stress test thus able to both expose dormant cardiovascular disease or change the cardiovascular system, inflammatory profile, and endothelial function [82]. These, in turn, make women predisposed to HF. Peripartum cardiomyopathy in particular occurs in the final month of pregnancy or months post-partum without any other known causes of HF and can be life-threatening [82]. Takotsubo cardiomyopathy is an acute condition with very uneven sex distributions. It has a female to male ration of 9:1 [82]. More than half of Takotsubo patients had a chronic neurologic or psychiatric disorder however the exact pathology of this condition is unknown [82].

### Diagnostic Standard

While it is understood that the female heart varies from the male heart and the intricacies in how it is neither simply proportionally smaller nor the same, sex-specific diagnostic standards are lacking. Since the female heart contains a 15 mm maximum thickness threshold in the walls regarding hypertrophy, it will have a higher extent of hypertrophy at the point of diagnosis than the male heart would [77]. If the standard of diagnosis involved sex-specific assays, it could lead to 30% more female diagnoses and 4.9% more male for type 1 myocardial infarction [77]. Currently, HF is diagnosed via ejection fraction thresholds that neglect sex [77]. The criterion dictates that HF results in 50% preserved ejection fraction while women have a higher baseline ejection fraction than men [77]. When the same criteria are used regardless of sex, it leads to detrimental impacts on women who are diagnosed later, and once HF is significantly more progressed than in their male counterparts. To better diagnose, the preserved ejection fraction threshold should be held much higher than 50% [77].

### Fibrosis

Cardiac fibroblast (CF) cells arbitrate fibrosis. A study found that the female subjects both clinically and in animal models had less cardiac fibrosis than males [98]. This discrepancy can be attributed partially to ovarian hormone 17β-estradiol (E2) [98]. This hormone signals using estrogen receptors in order to mediate the genes involved in the fibrotic response and transition to myofibroblast transition [98]. E2 mediates microRNA transcription which regulates fibrosis in the heart [98]. During stresses such as HF, the CFs emit massive amounts of collagen into the extracellular matrix [98]. Different fibrosis patterns occur depending on the type of cardiovascular disease however the development and deposition emerges later in the remodeling process in females than males [98]. Women with dilated cardiomyopathy present with less fibrosis and apoptosis, thus demonstrating clinically an increased reversible HF type [98].

### Cardiac Mechanical Stresses and HF

### Mechanical Activation of Cardiac Fibroblasts

The heart is comprised of five major cell groups which are fibroblasts, cardiomyocytes, macrophages, smooth muscle cells, and endothelial cells [99]. Cardiac fibroblasts are the main cells that deposit the extracellular matrix in the heart by regulating matrix metalloproteinases and their tissue inhibitors [99]. Consequently, collagen secretion and degradation are moderated [99]. It supplies structural support for the myocardium as well as playing a role in several signaling processes [99]. The heart has finite regenerative capabilities after being injured [100]. Consequently, repair processes entail discarding necrotic cardiomyocytes after fibrotic scar tissue replacement. This serves the purpose of maintaining the integrity of the myocardial function and structure [100]. When cardiac injury occurs, cardiac fibroblasts develop into myofibroblasts and significantly aid in cardiac remodeling [99]. Myofibroblasts are crucial for structural integrity maintenance in the myocardium and so by contracting the surrounding tissue, ultimately remodeling the scaffold of the ECM [101]. While necessary, excessive fibrosis caused by constant myofibroblast activation leads to a tissue compliance progressively diminishing [101]. This leads to decreased nutrient and oxygen delivery as well as greater cardiomyocyte atrophy and cell death [101]. These ultimately result in intensifying left ventricular dilation and dysfunction inevitably leading to heart failure [101].

### Mechanical Forces Sensed by Cardiac Fibroblasts

Cells are able to sense their environments which enables them to respond to mechanical cues and adapt [102]. Those mechanical cues are brought by the ligand and received by the receptor at the binding interface [102]. This occurs in four main steps [102]. The first step is mechanopresentation where the mechanical cues are presented for the cell to sense it. This needs a ligand secured on the surface to withstand force that is exerted on the receptor [102]. Following that is mechanoreception where the mechanopresenting ligand engages with a cell surface receptor [102]. That mechanoreceptor could change the receptor's binding site or the ligand, ultimately changing the properties of the bond [102]. The next step is mechanotransmission that is carried out by the mechanotransmitter by which the signal migrates away from the binding site of the ligand and head towards the inside of the cell [102]. The final step is mechanotransduction which is where the mechanical signal undergoes translation into a biochemical signal [102].

Paxillin is an adapter protein that is involved in integrin mediated cell adhesion to the ECM [103]. This protein has been connected to signaling pathways and because of its vast range of interactions with proteins, it has been shown to be involved in many mechanisms [103]. It was investigated because it is a marker for focal adhesions (FAs) and is necessary for heart function [103]. A deficiency in paxillin results in destabilization of binding partners and later, accelerates heart failure [103]. Focal adhesions are large, multiprotein complexes [104] that act as a mechanical link between the cell and the environment and serve as mechanosensors [105]. Cells remodel and organize focal adhesions when mechanical forces are present [105]. They allow for adaptation by adjusting the contractile activities, stability, and growth [105]. Furthermore, FAs have shown to be sensitive to strong forces and the process of assembly and disassembly of the proteins are influenced by the ECM's rigidity as well as the existence of outside forces [105]. FAs self-assemble and elongate once pulling forces are exerted and separate once the forces decrease [104]. The molecular aggregates that are under force grow in the direction of that force by involving more molecules [104]. Mature adhesions are characterized by additional active processes which further increase the mechanical load of FAs and the buildup of stress fibers [104].

### Goal and Future Direction

Cardiac cells are dynamic physical structures that constantly encounter mechanical stimuli, such as dynamic forces and mechanical features of the extracellular matrix (ECM), in their environment [106]. These mechanical cues are transduced into biochemical signals and integrated with genetic and chemical signals to modulate cardiac homeostasis [106]. Although it is well known that mechanical stresses sensed by cells result in a broad variety of cellular responses [106], the molecular mechanisms underlying this ability, including the mechanoreceptors which receive mechanical stress and convert stresses into intracellular biochemical signals, and how cardiac cells (such as cardiomyocytes, macrophages and fibroblast) interact with each other under pathological and physiological conditions, and how these biological responses differ between sexes are still poorly understood. This can be credited due to the lack of proper in vitro systems that can recapitulate cardiac injury, such as tension and compressional stresses that occurs with cardiac damage and recovery [106]. Thus, a greater discernment of these mechanisms is fundamental to better understand the development and progression of various sex-specific cardiac pathologies in which a dysregulation of the mechanosensing mediated pathways is often involved. The ultimate, long-term goal of this investigation is to better understand and identify sex-specific molecular mechanisms that trigger fibrosis with the main goal to engineer sex-specific therapies.

Literature Review of the Techniques and Methods Used in this Investigation

### Cell Selection (Fibroblast)

Fibroblasts were extracted from isolated from C57BL/6-2 month-old mice (female and male) using the Langendorff method. This method is frequently used for hearts whose aortas are not only large enough for cannulation but also miniscule enough for controlling enzymes such as rabbits, guinea pigs, and mice [107]. The enzyme solution usually flows through the aortic cannula. For larger hearts such as human, canine, or porcine it is more efficient to cannulate and perfuse a major coronary artery, or a subdivision, and then the perfused region cut out for advanced digestion [107].

### Cell Stressor

The molecular mechanisms by which damaging mechanical stressors activate pathways that drive fibrosis in the heart and how these biological responses differ between sexes are still not entirely determined. This is in part attributed to the lack of proper in vitro systems that can recapitulate cardiac forces and injury, such as tension and compressional stresses that occur during cardiac damage and recovery. A novel device was engineered to study the effects of mechanical stresses on cells: a cell stressor which applies both tension and compressional stresses simultaneously. Integrated research activities focused on engineering models of cardiac mechanical stresses that occur during pathological and physiological conditions were utilized for investigating sex-specific molecular mechanisms that trigger fibrosis. The design of the cell stressor was based on a 4-point bend test as outlined by the standard test method ASTM E855 08, as discussed in detail above with reference to FIG. 6.

The cell stressor device (e.g., as described in detail above with reference to FIG. 8) developed by the Pena lab includes the main body, bender, screw, shelf, and the beam. Since the cells were alive, this process was done in a fume hood. A torque screwdriver set to 5.1 Nm in addition to a cell stressor device set to that force. The beam material is Polymethyl methacrylate (PMMA) beam and is coated gelatin-fibronectin (GF). The body of the device is 3D-printed acrylonitrile butadiene styrene (ABS). Unlike other commercial devices, the cell stressor can 1) apply tensile and compressive stresses simultaneously to cells 2) recapitulate the native cardiac structure by ECM proteins in the PMMA beam and 3) be used to assess real time cell imaging. These features allow an investigation into mechanical cellular alterations in real time enabling experiments that have not been possible in other in vitro cardiac models.

A co-culture of NRVMs (cardiomyocytes and fibroblasts) were initially used to assess stresses on cells. A co-culture of cardiomyocytes and cardiac fibroblasts were exposed to tensile and compressional stresses for 30 min to obtain preliminary results. Aside from a lower cell count following the stress (fewer cardiomyocytes), it was observed that the fibroblasts went from being thin in the control to being larger following stress. An increase in fibroblast size and signal intensity is a marker for its myofibroblast phenotype [108], otherwise known as the “activated” fibroblast. As controls, cardiomyocytes and fibroblast were used without applying stress. After the stress, it was observed that 1) a morphological disruption occurs in the cardiomyocytes and 2) an increased number of enlarged fibroblasts, most likely activated (pathological). This demonstrates that this device can recapitulate what happens in the injured myocardium in which cardiomyocytes are lost and fibroblast take over to start a pathological cardiac remodeling by creating a scar to support the injured myocardium [105].

### Gelatin-Fibronectin Beam Coating

In previous experiments done by the Pena lab, the beams were coated with gelatin. A shortcoming of this is that cell adhesiveness of gelatin is not sufficiently strong. Fibronectin is an adhesive glycoprotein [109]. It is able to bind to a variety of ligands which includes cell surface receptors and collagen [110]. As a result, a gelatin-fibronectin solution was made to coat the beams since fibronectin can firmly bond with gelatin [109]. A PMMA beam had its film removed for beam transparency prior to UV treatment and gelatin-fibronectin coating.

### Confocal Microscopy

Confocal microscopy is widely used in many fields such as pharmaceuticals, biologics, medical, and material research [111]. Confocal microscopy was patented in 1957 by Dr. Marvin Minsky [111]. This technology significantly enhances optical image resolutions [111]. The illumination and detection optics are focused on the same diffraction-limited spot which moves over the sample to compile a complete image on the detector [112]. A principle function of the confocal microscope is to provide a source of light and reject out-of-focus light [112]. Thus, samples such as tissues are able to be deep imaged with high resolution [112]. This patented idea was achieved by utilizing illumination and detection-side pinhole apertures in the same conjugate image plane and subsequently, making them “confocal” [112]. One pinhole was positioned in front of a zirconium arc light source to create a point of light focused on the sample by an objective lens. The second objective lens focuses the illuminated sample point onto the second pinhole in front of the detector. This “double focusing” system denies the out-of-focus rays from the illuminated sample, so they do not reach the detector. The stage can be moved in x, y in order to scan the sample through the illumination point to finally build the final image [112].

A critical, unique feature of the confocal is it capability to provide narrow, focused images by a true means of depth-wise optical slicing [111]. As a result, 3D reconstructed information can be acquired from the line-of-sight depth-wise resolved imaging without requiring the physical slicing of specimens [111]. Overall, the confocal provides clearer, more vivid images. The confocal was able to show significantly more details such as cardiomyocyte striation, whereas the EVOS allowed for each image color to be taken separately.

### Atomic Force Microscopy

Atomic force microscopy (AFM) is a 3D topographic method with a high atomic resolution that measures surface stiffness [113]. AFM detects forces acting between a sharp tip and the surface of a sample [113]. The AFM can be modified to assess many properties like adhesion forces, friction, Youngs modulus, and magnetic properties. It is capable of analyzing polymers, films or fiber [113], as well as cells. It is a type of scanning probe microscope (SPM) which utilizes a fine probe over the surface instead of using electrons or beam of light [113]. The contact mode is a frequently used mode of the AFM [113]. The AFM consists of a cantilever that holds a fixed probe which indents or scans the sample [114]. The laser beam pointing on the cantilever surface can be reflected and collected by a matrix of photodiodes [114]. The cantilever bends when the probe comes into contact with the specimen [114]. The deflection is detected as a variation of currents transduced by photodiodes [114]. The probe tip is frequently made of silicon and silicon nitride [114]. The cantilever system can be mad of specific materials depending on its desired customizations [114].

### Cell Staining

Cell staining is a procedure that allows for the visualization of cells using a microscope. This is one through the utilization and combination of specific antibodies. In other words, it is accomplished by first exposing fixed cells to primary antibodies that are directed against one or more proteins of interest and then the bound antibodies are detected using secondary antibodies directed against the invariant portion of the primary antibody [115].

### Mechanobiology

### Piezo1 Ion Channel

Mechanosensitive ion channel Piezo1 appears to be linked to many diseases and could be upregulated in cardiac fibroblasts as a result of mechanical stresses and pro-inflammatory stimuli post-myocardial injury [116]. A greater Piezo1 activity could be behind the positive feedback loop that induces fibrosis progression [116]. Since it triggers actin stress fiber formation, the increased Piezo1-mediated calcium flow could be significant in cytoskeleton reorganization [116]. That fiber formation is an established characteristic of fibroblast transdifferentiation into the myofibroblast, the activated form of the fibroblast [116]. Piezo1 was discovered in 2010 and noted its expression in many mammalian cells and utilized to convert mechanical force to biological signals [116]. It is a non-selective channel which is activated by pressure and is the only channel to be gated by mainly mechanical stimuli rather than chemical signals [116]. Piezo1 was noted in the nucleus, mitochondria, and endoplasmic reticulum [116]. This ion channel has also been labeled as a critical player in aortic valve development. There could be a correlation between Piezo1 and cardiovascular diseases due to its expression in cardiac fibroblasts and cardiomyocytes [116]. Furthermore, Piezo1 could also be a biomarker for labeling the progression of cardiac fibrosis [116]. However, since Piezo1 is newly discovered, further studies need to be done to elucidate its role in fibrosis [116].

### Mitochondria

Many mechanisms have been reported to be crucial for myofibroblast formation [101]. The mitochondria regulate apoptosis by releasing apoptogens [101]. However, the myofibroblast is incredibly resistant to cell death which is how it is able to survive in fibrosis [101]. Changed fibroblast mitochondrial function during fibrosis likely leads to apoptotic resistance and proliferation. The activated fibroblasts during remodeling counts on the preservation of mitochondrial integrity such as its function and structure [117]. Researchers hypothesize that the molecular mediators of bioenergetics, mitochondrial ultrastructure as well as reactive oxygen species (ROS) [117]. Currently, there is opposing proof on whether oxygen consumption during CF activation increases or decreases [117]. Similarly, there has not been sufficient investigations into sex differences in cardiac fibroblast cells.

### Research Objective and Experimental Approach

### Hypothesis

Preliminary studies (e.g., as discussed above with reference to Example 1) showed that cells react to mechanical stresses. As a result, we hypothesize that the effects of compressive and tensile mechanical stresses on cardiac fibroblasts will vary between the sexes.

### Specific Aims

Aim 1: Exert pathological in vitro mechanical stress on male and female cardiac fibroblast to assess how those stresses affect the cells in a sex-specific manner.

The cytoskeleton will be assessed via phalloidin staining, cell count through DAPI, and fibroblast activation via smooth muscle actin. Paxillin will also be investigated by stainings as well to identify focal adhesions. In doing so, it can be observed with immunofluorescence staining and AFM the effects of mechanical stresses on the fibroblasts.

Aim 2: Investigate sex-specific mechanobiology of fibroblast with particular focus on ion channels and mitochondria function.

Piezo1 ion channel and mitochondria function will be of particular interest and thus a Piezo1 staining along with mitochondria staining will be performed in order to seek potential answers to why the fibroblasts react in their particular manner to mechanical stimuli.

### Aim 1: Exert Pathological In Vitro Mechanical Stress on Male and Female Cardiac Fibroblasts to Assess how Those Stresses Affect the Cells in a Sex-Specific Manner Methods for Aim 1

### Beam Cutting

The beams used for this study were laser cut from PMMA at InWorks on the University of Colorado Denver downtown campus. In previous studies, there were several shortcomings of the manner in which the beams were prepared. Firstly, it used to be cut without the plastic film on both sides. This resulted in a powdery substance on the beams which made it appear cloudy when under a microscope. Consequently, the only way to observe the cells on the beams was to stain with antibodies then check on the Evos microscope and/or confocal. This made it difficult to assess if the cells had adhered to the beam prior to stressing. The powdery substance also could not be eliminated by washing with soap and water, but rather, could only be wiped by ethanol. Unfortunately, beams cleaned with ethanol snap and break down the middle immediately upon stress application in the cell stressor. As a result, the PMMA slab was laser cut with the plastic film intact on both sides. The film was peeled off with tweezers prior to preparation. Secondly, previously cut beams did not have a vertical line etch nor an etched ‘T’ which made it difficult to determine which side the cells were seeded on when stressing the cell since the beams flip frequently. Once the adjustment of a line with the ‘T’ was added, it made it simpler to remember the correct orientation (top surface) of the beam is when the ‘T’ is on the right side of the line for gelatin-fibronectin coating, cell seeding, and cell staining. The resulting arrangement had 378 beams per PMMA piece, wherein individual beams had a line down the middle which gets an etch. It also had a ‘T’ on the right side of the line in order to seed the cells on the correct side of the beam.

### Beam Preparation

The beams were first exposed to UV light for 15 on each side prior to coating in a 100 mm tissue culture dish. In order to prevent the cells from spilling off the beam, they were coated with a gelatin-fibronectin solution. The solution was made by weighing 0.1 grams gelatin and placing into a 500 mL glass bottle. Distilled water was added until the 500 mL mark and autoclaved. This makes the gelatin concentration 0.02%. Then, 1 mL of liquid fibronectin was diluted in 199 mL and gently mixed. Immediately after, aliquots of 6 mL per 15 mL centrifuge tubes were prepared and frozen at −20 degrees C. When needed, the one of the aliquots were thawed in a bead bath and 100 μL were added onto each PMMA beam carefully to prevent the rupture of surface tension that would result in the gelatin-fibronectin solution spilling off the beam. Once the beams were coated with no surface tension breakage, the beam with its plate were placed in the incubator for one hour. Finally, they were placed in the refrigerator until the cell seeding. This aspect of the methods began with UV sterilization of the beam, then incubation and then storage in the refrigerator.

### Fibroblast Isolation

Female and male cardiac fibroblast cells were isolated from C57BL/6-2 month-old mice. The perfusion buffer was made prior to the isolation. The surgical station and perfusion apparatus were prepared by turning on the circulating water bath and setting the temperature to 37° C., preparing the centrifuge at 4° C., and cleaning perfusion system by flushing with 70% ethanol and double-distilled water (ddH2O). The perfusion system was loaded with perfusion buffer (including the inner column) by attaching the 1.0 mm glass cannula filled with perfusion buffer to the holster and securing underneath a dissecting microscope in a 10 cm petri dish lid filled with perfusion buffer. 200 μL of heparin was administered via intraperitoneal injection for 20 minutes before beginning the anesthetization.

The mouse was anesthetized with isoflurane and a cervical dislocation was performed. Each of the mouse's limbs were secured on the surgical platform with pins. The chest was disinfected with 70% ethanol, lifting the skin with forceps just below the sternum. A lateral incision was done with small scissors, cutting through the skin and abdomen. The diaphragm was removed and cut through ribcage. The pericardium was removed as well. While gently lifting the heart using curved forceps, a single cut was made through the major vessels leaving enough aorta to make cannulation easier.

The heart was placed in a petri dish containing perfusion buffer to wash away blood. Then, placed into a petri dish containing perfusion buffer under dissecting microscope. The aorta was sheathed onto the cannula using fine forceps such that the tip was just distal to the aortic valve careful that the cannula does not puncture the aortic valve otherwise the heart will not properly perfuse. The aorta was secured on to the cannula using a small clip. The heart was then transferred to the perfusion system. The aorta was secured around the cannula with a pre-tied 7-0 silk suture but not tied below the tip of the cannula or the aorta will be completely occluded.

The heart was perfused at 4 mL/min for all steps beginning with perfusing with perfusion buffer. During this time, the collagenase was resuspended in 50 mL of perfusion buffer (Digestion Buffer). 12 mL was transferred to the heated tissue bath. 19 μL of 100 mM calcium chloride (CaCl₂)) was added to the remaining 38 mL. It was perfused for 3 min in digestion buffer without CaCl₂). Then, it was perfused with digestion buffer containing CaCl₂) for about 9-10 minutes. Once the digestion buffer emptied, it was switched back to the perfusion buffer for an additional 5 minutes to clear the remaining digestion buffer in the system.

The heart was removed from the cannula and placed in a 60 mm dish containing Digestion Buffer with CaCl₂). The extra-ventricular tissue was removed using fine scissors. The tissue was mechanically disrupted using transfer pipettes. Once there were no longer any large pieces remaining, it was gently pipetted up and down using a transfer pipette until the tissue was completely dissociated. The cell suspension was filtered through a 250 μm mesh into a 50 mL conical tube. The dish was washed with Stop Buffer and filtered until the volume reached 10 ml. The cell suspension was centrifuged at 30×g (360 rpm) for 3 minutes to pellet the cardiomyocytes and collect the supernatant. The cell suspension was filtered through a 40 μm cell strainer and centrifuge at 500×g (1,480 rpm) for 5 minutes at 4° C. to pellet the non-myocytes.

To remove debris and erythrocytes, the non-myocytes were washed in 10 mL DPBS+0.04% BSA and centrifuged at 500×g for 5 minutes. The non-myocyte pellet was resuspended in 3.1 mL cold DPBS. 900 μL cold Debris Removal Solution was added and pipetted gently. It was gently overlayed with 4 mL cold DPBS not allowing it to mix, centrifuged at 3,000×g (3,630 rpm) for 10 minutes at 4° C., and aspirated away the top two phases (leaving about 1 mL). The tube was filled to 15 mL with cold DPBS and inverted three times to mix, and centrifuge at 1,000×g (2,100 rpm) for 10 min at 4° C. Then, the supernatant was aspirated. The cell pellet was resuspended in 10 mL cold erythrocyte lysis solution and centrifuged at 500×g (1,480 rpm) for 5 minutes at RT. The cells were washed in 10 ml DPBS+0.04% BSA, centrifuged at 500×g for 10 min, and resuspended the non-myocytes in ˜500 μL cold DPBS+0.04% BSA.

Then, the cells were plated in a tissue culture dish labeled with its respective sex along with fibroblast media (89% Dulbecco's modified eagle medium, 10% bovine calf serum, and 1% antibiotic-antimycotic). The male and female cells were incubated for one hour to allow the cells to adhere to the tissue culture dish. The excess media was aspirated, and the cells were washed with new fibroblast media. Finally, the cells were resuspended in 10 mL of fibroblast media and incubated until the following day where a media change would be done.

### Cell Seeding

Media washes were done 24 hours post-isolation and then every 48 hours for 1 week until the cells were confluent. At the 1-week mark, the cells were split. They were counted using an automated cell counter. While the cell solution was centrifuging for 10 minutes during the seeding process, the leftover gelatin-fibronectin liquid solution on each beam was aspirated and allowed to dry in the fume hood. The beam being coated but also now dry allowed the cells to adhere more easily due to surface tension, thus preventing the cells from spilling off the beam and onto the tissue culture dish. Once dry, the beams were transferred to a labelled 6-well plate with up to 2 beams per well. The cells came from two mice (1 male and 1 female) per experiment resulting in a total of 3 experiments (6 mice—3 males and 3 females). Each batch of beams corresponded to one mouse (cells from different mice were not combined). When the cell count was obtained, 42,000 cells were seeded per beam carefully to prevent surface tension rupture (100 mL) and incubated until the next day. Then, the 100 mL of media cell solution per beam was aspirated and 3 mL of media was added per well and incubated until the next day where the beams would be stressed.

### Cell Stressing

Prior to cell stressing and gelatin/fibronectin coating, the devices were exposed to 254 nm UV light for 30 minutes on each side. Then, in the biosafety cabinet, the device was placed in a tissue culture plate filled with fibroblast media, enough to cover the beam (˜5 mL) so that the cells are only suffering due to stress and not lack of nutrients. The media was comprised of 89% DMEM, 10% bovine serum, and 1% antibiotic-antimycotic. Beam stressing was done in batches since there are 5 total devices and 8 or more beams at a time to stress. The beams were placed onto the shelves of the cell stressor with tweezers then held the bender and the body of the device together to prevent the bender from slipping out when applying the stress. Once secure, the torque screwdriver was set to 5.1 Nm and used to twist the screw until that point, thus exerting stress onto the beam. Then, the device with beam in the media-filled tissue culture plate was placed in the incubator for 30 minutes. Immediately following the stress, the beam containing the cells on the surface was removed from the cell stressor device and either 1) fixed or 2) kept in media for stress recovery experiments. The beam was washed with PBS 3 times and fixed with 4% paraformaldehyde for 15 minutes before finally being fully submerged in PBS. As such, 42,000 of fibroblast cells were seeded on to the beam with a P200 micropipette and then that beam was inserted into the UV-sterilized cell stressor.

### AFM

To assess mechanical properties, atomic force microscopy was performed on individual cells from sets of beams from each category (female stress, female control, male stress, and male control). A control was measured while another beam with its respective sex was stressing in the incubator for 30 minutes. Once finished with the first control and immediately after the other beam was done stressing, this second beam was examined with the AFM. Then, another control was done followed by another stress in the same fashion. Force Spectroscopy mode, was used also to determine the nanomechanical properties of the cells. During acquisition, MLCT AFM probes were used, these have a pyramidal tip made of Si3N4, with a curvature radius of 35°, manufactured by Bruker®. The cantilever spring constants were systematically measured using the thermal tune method and has been found to be in the range of 0.01 to 0.02 N/m. The maximal force applied to the tissues has been limited to 1.5 nN in order to preserve the tissue integrity and with 5 μm of Z piezo. At least 20 cells per groups were analyzed. For both methodologies, all the physical and physiological cues regarding AFM analysis and sample preparation were kept constant in all the samples. The Hertz model was used to determine the mechanical properties of the cells using the JPK software.

### Stress Recovery

A batch of beams from each category (female stress, female control, male stress, and male control) were saved post-stress and not fixed to assess its recovery capabilities. Consequently, after the stress, the beams were taken out of the device and placed in a labeled 6 well plate with 3 mL media in each well. Then, it was placed in the incubator for 48 hours. Following that, the beams with cells were fixed washed with PBS three times and submerged for 15 minutes in 4% PFA to fix the cells. This process for assessing the stress recovery thus included removing the beam from the cell stressor device according to the present technology, and then placing it in cell media for 2 days of incubation in the incubator. Following that, the beams with cells were stained with antibodies to observe the effects of stress and if/how the cells are able to recover.

### Cell Staining

Once ready to be stained, the PBS was aspirated, and the beam with cells was submerged in 1% triton x in PBS by volume for 1.5 hours to permeabilize the cell membranes. Upon time completion, the triton x solution was aspirates and the beam was covered with 2% BSA in PBS solution for 45 minutes to block nonspecific antibody binding. Then, the BSA was aspirated, and the cells were washed twice with PBS and a final time with 1% triton X.

To assess the cytoskeleton of the cells, a phalloidin conjugate was used. While it is not an antibody, it is a peptide with a high affinity for actin. As a result, a 1:1000 solution of phalloidin in 2% BSA were treated to the beam with cells for 1 hour. Then, a 1:8000 4′,6-diamidino-2-phenylindole (DAPI) in PBS solution was added for 2 minutes to stain the nucleus, and the cells were submerged in 3 mL of PBS. The beams were kept at 4° C. until ready for imaging. α-Smooth muscle actin (SMA) was also utilized to identify and observe fibroblast activation and stress fiber formation [118]. Furthermore, α-SMA plays an important part in myocardial remodeling [118]. A 1:200 α-SMA in BSA solution was made to examine these features with Alexa Fluor 488 as a secondary antibody.

A 1:50 paxillin solution was made for a batch of beams (always consisted of 1 female control, 1 male control, 1 female stress, and 1 normal stress). A 1:200 solution for the Piezo1 ion channel was also created for another beam batch. All antibodies used and their information including secondaries and ratios can be found in Appendix L, below.

### Beam Imaging and Quantification

Upon staining, the beams were imaged with the confocal microscope. The images were processed using the ImageJ software where the separate channels were merged into one image. Cell count was obtained by manual counting on ImageJ using its counter feature. Paxillin expression (paxillin-positive area) was quantified through the macro feature on ImageJ with sample code shown in Appendix N, below. Once that was performed, the value was normalized for cell count. These were done individually per confocal image and all data was kept on a spreadsheet. MATLAB code was developed to measure the intensity of the signal from SMA, as found in Appendix 0, below. This code includes built-in cell normalization.

### Statistical Analysis

All statistics were done on the raw spreadsheet data using t-tests comparing the means of two groups to assess significant differences in the results. All the experiments were performed in triplicates from at least three independent cell isolations.

### Results and Discussion for Aim 1

### Effects of Mechanical Stresses on Cytoskeleton Arrangement and Focal Adhesions

Phalloidin was used to stain the cytoskeleton of the cell (red), paxillin was used to assess the focal adhesions (green), and DAPI to stain the nucleus of each cell (blue). Both the female and male samples under no stress were confluent with several cells, each with a healthy extracellular matrix. The filaments of each cell could also be visualized in red. However, the difference was most notable when the cells were exposed to pathological stress.

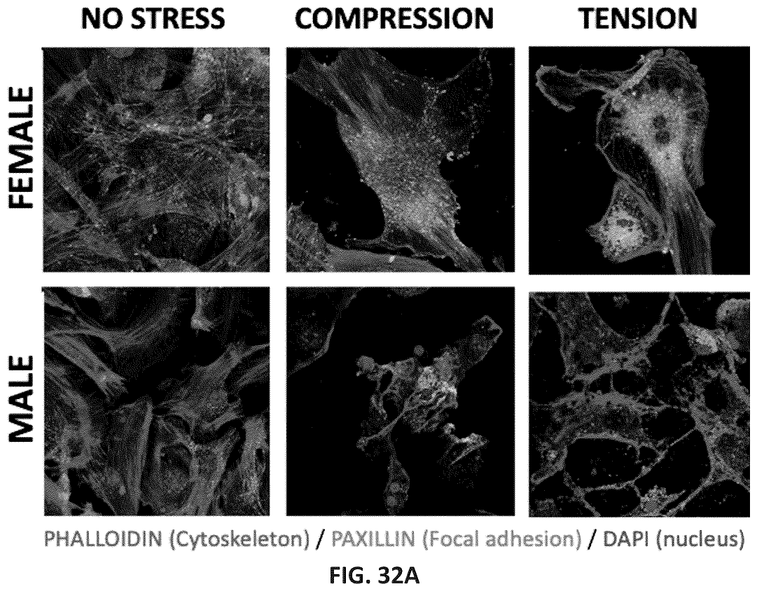

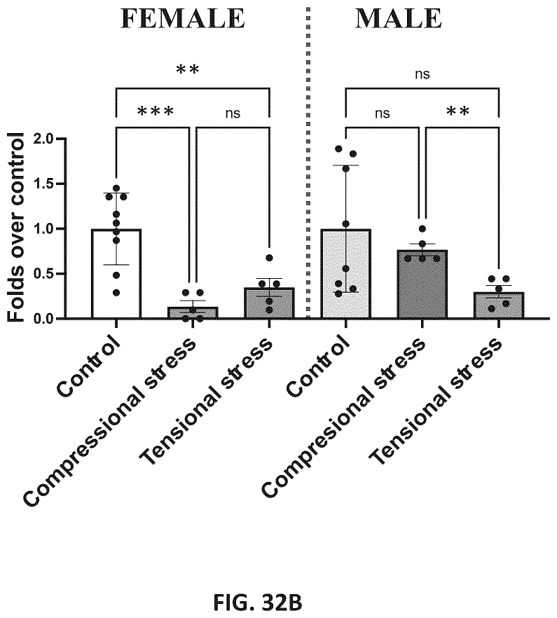

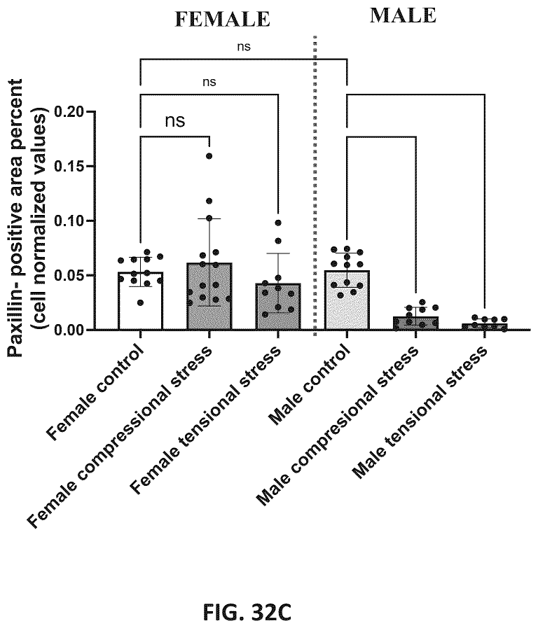

The male compression and tension images demonstrated the cell cytoskeleton to be quite damaged and the fibers were diminished. The female compression and tension images showed more defined fibers than the male stresses and a healthier-appearing cytoskeleton that preserved more of its normal flat and spindle shape [119] than the male, as shown in FIG. 32A. In FIG. 32A, what is demonstrated through the images is phalloidin, paxillin, and DAPI staining on female and male cells under no stress, compressional, and tensional stresses with a scale of 33 μM. Though fewer in cell count than the male, the female cells were able to maintain a more favorable cytoskeleton than its male counterpart.

FIG. 32B displays quantification of cell count as folds over control done over the course of 3 sets of experiments. A cell count was also performed before and after stress. In females, control versus tensional stress was found have significant differences (p-value<0.01). The female fibroblast was reduced by 5-fold after compressional stress and 2.5-fold after tensional stress when compared to their control (non-stressed sample). The female control versus compressional stress also had significant differences (p-value<0.001). However, it was observed that when comparing the female compressional stress with female tensional stress that there was no significant difference. The male control was found to have no significant difference compared to tensional stress as well as compressional stress. Male compression versus male tension had significant differences (p-value<0.01). The male fibroblast under compressional stress was reduced by 2-fold compared to tensional stress. In FIG. 32B, the cell count is shown as folds over control showing control, compressional stress, and tensional stress in females and males.

FIG. 32C displays the paxillin-positive areas in percentages for each of the categories. In FIG. 32C, paxillin-positive area percent normalized based on cell count for control, compressional, and tensional stresses in females and males). ‘ns’ means no significant difference, **** p-value<0.0001, *** p-value≤0.001, and ** p-value≤0.01 through t-test with total n=97. It can be observed that there was no significant difference between the male and female controls and also, there were no differences between female controls and female stress samples (both tension and compression). However, there were significant differences between the male controls and both male stress samples. The male fibroblasts under compressive and tensional stresses showed a very low percentage of paxillin whereas the females exhibited much a higher percentage for those two types of stresses. Thus, the females had more paxillin, which as previously mentioned, is necessary for heart function [103]. This also indicates that the male focal adhesions complexes were less mature and weakly attached. This is assuming all the cells stuck to the beam and did not fall off. Future experiments could establish how many cells are lost during this process by slipping off the beam.

### Fibroblast Activation

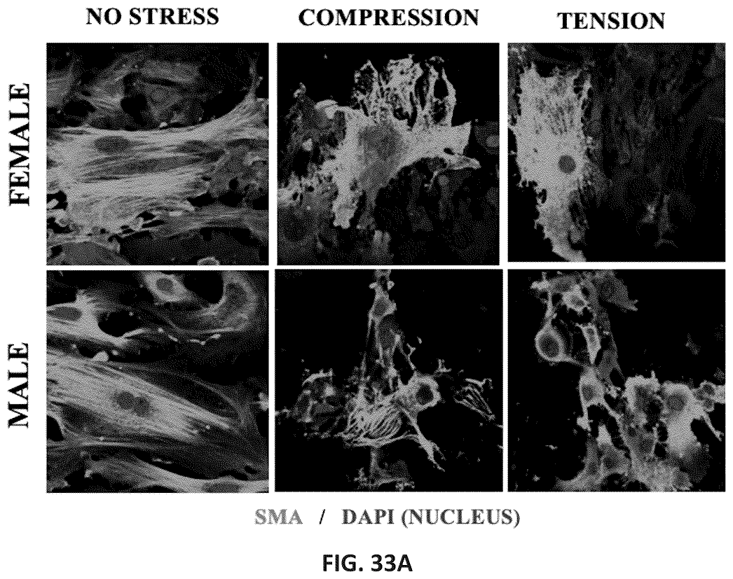

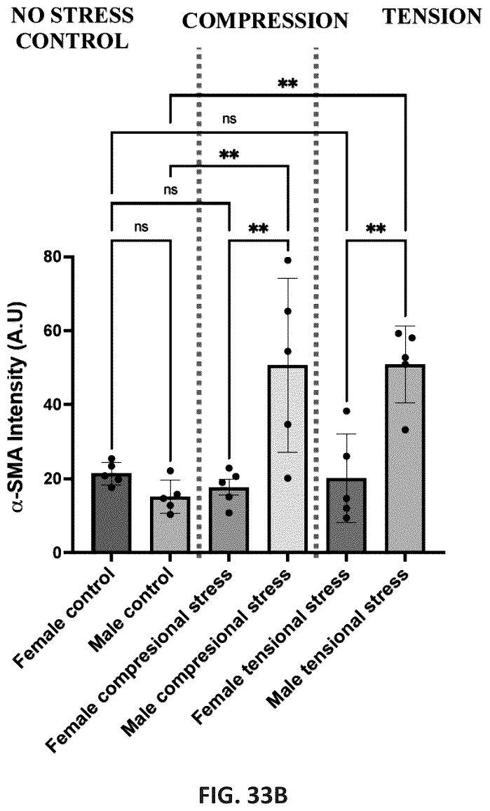

While fibroblasts were already activated by the slightest stimuli (beginning once isolated and cultured), it was deemed to be significant to assess whether mechanical stresses further activate fibroblasts in comparison with its control. Subsequently, α-smooth muscle actin staining was performed in conjunction with DAPI to observe fibroblast activation and cell count with and without mechanical stress. FIG. 33A displays the comparisons between the sexes as well as their treatment conditions (control versus compression versus tension). The female and male controls show elongated fibroblast cells with individual actin filaments visible as well as several cells. In FIG. 33A, SMA and DAPI stainings on female and male fibroblast cells are shown in the images under no stress, compressional, and tensional stresses with scalebar of 30 μM. Upon stress, there were differences in α-smooth muscle actin intensity as shown in FIG. 33B.

FIG. 33B quantified the intensities of the α-SMA normalized per cell. There was no significant difference between the female and male controls indicating that females and males contained similar levels of fibroblast activation. In FIG. 33B, quantification of the SMA intensity comparing the female and male fibroblasts cells in each category (no stress, compression, and tension). ‘ns’ means no significant difference and **=p-value≤0.01 conducted through a t-test with a total n=109. The comparison between female and male cells under tensional stress as well as compressional stress is statistically significant (p-value<0.01). The male fibroblast cells had higher α-SMA intensities after both types of stresses than its female counterpart. This corroborates that α-SMA intensities vary by sex when they are under compressional and tensional stresses. More specifically, in a study done by Peter et al. in which Isoproterenol was administered to rats to model cardiovascular hypertrophy in males and females, it was observed that male cardiac fibroblasts were more activated to myofibroblasts than females [120]. This indicates that our in vitro model of mechanical stresses can recapitulate an in vivo model of cardiac hypertrophy.

### AFM

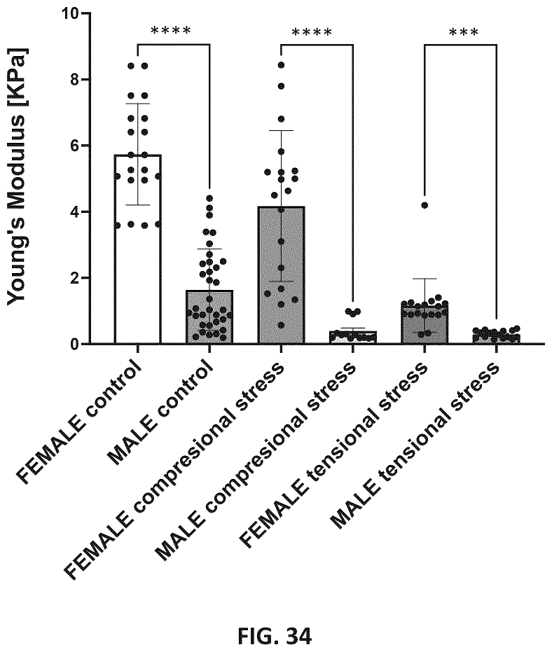

In order to highlight the mechanical properties of cardiac fibroblast cells in conjunction with sex differences, atomic force microscopy was done in all the samples. When comparing each female category to its respective male category, it yielded highly statistically significant results in all three comparisons. The Young's Modulus of the male fibroblast control was significantly lower when compared to the Young's Modulus of the male fibroblast versus the Young's Modulus of the female after stress. This is depicted in FIG. 34, which also provides the statistical analysis with **** p-value≤0.0001 and *** p-value≤0.001. Overall, the female had a higher Young's Modulus, denoting higher stiffness of female cells. More specifically, the highest stiffness can be observed in the female fibroblast control beam correlating with literature in how the left ventricular (LV) stiffness is higher for women in both the systolic and diastolic [81].

### Stress Recovery

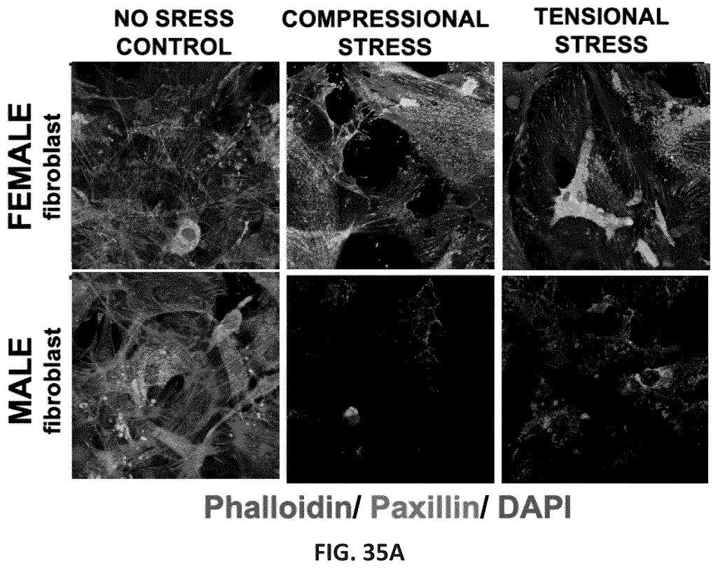

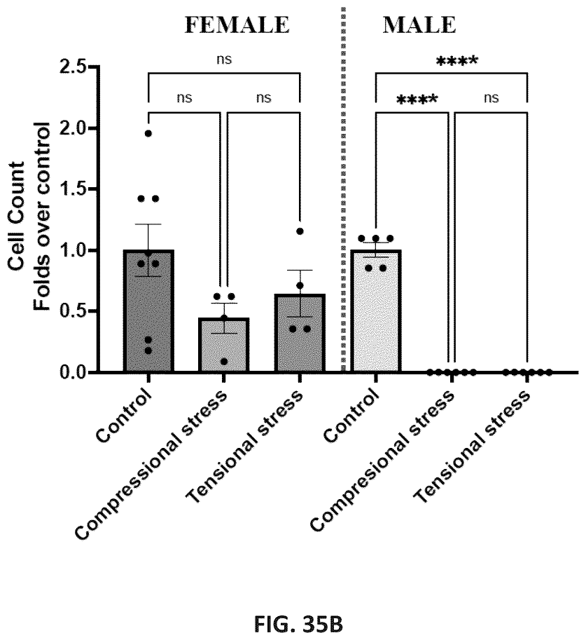

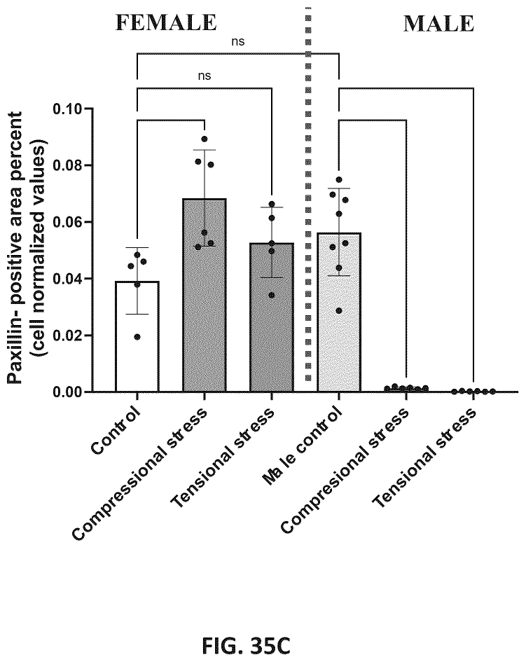

While differences were observed in how female and male fibroblasts react to mechanical stresses, it was a point of interest to allow the cells to recover and determine if there is a variation in recovery as well. FIG. 35A depicts the cells after 48 hours of recovering from 30 minutes of stress. In FIG. 35A, confocal images are provided of phalloidin (red), paxillin (green), and DAPI (blue) stainings on recovery beams with scale of 33 μM. The beams with cells were stained with phalloidin, paxillin, and DAPI. Both the female and male fibroblasts under no stress show a large number of cells with abundant actin fibers. After stress recovery, the female fibroblast proliferated with cells showing a recovered cytoskeleton. More importantly, there were no significant differences regarding cell count between the female controls and the stress-recovery fibroblast (both under tension and tension).

On the contrary, there was no DAPI expression in the male compression and tension denoting that the cells were unable to recover post-stress and, consequently, died. FIG. 35B depicts the cell count of each category. In FIG. 35B, cell count is provided in recovery beams for controls, compressional stress, and tensional stress in females and males. This ineptitude for male cells to recover aligns with the study previously mentioned in which Isoproterenol was administered to rats to create a cardiovascular hypertrophy model [120]. This is a “stress” model and ISO-induced cardiac hypertrophy represents the most frequently utilized model which constitutes a crucial indication of the pathogenesis of maladaptive cardiac hypertrophy [121]. It was found that ISO-treated males had higher mortality rates than the females [120]. The males developed fibrosis while the females did not [120]. When cardiac fibroblasts from both sexes were investigated, fibroblasts from the from the female hearts proliferated more than the male cells [120]; once more, our results demonstrate that out in vitro model of mechanical stresses can recapitulate an in vivo model of cardiac hypertrophy.

Paxillin was also studied and results in FIG. 35C demonstrate paxillin-positive areas as a percentage for each category. In FIG. 35C, paxillin-positive area percent is normalized based on cell count for controls, compressional, and tensional stresses in females and males. ‘ns’ means no significant difference, **** p-value<0.0001, ** p-value<0.01 done through t-test with total n=109. There was no significant difference between the male and female controls. However, a significant increase in paxillin was observed in the female compressional stress when compared with the female control. There was a tendency in the increase of paxillin in the female tensional samples when compared with the female control, however this was not statistically significant. However, this increase in paxillin could be due to male cells falling off the beams. Consequently, future experiments in which a comparison is done to note changes in cell count before and during experiments would assist in elucidating this.

Aim 2: Investigate Sex-Specific Mechanobiology of Fibroblast with Particular Focus on Ion Channels and Mitochondria Function

### Methods for Aim 2

### Piezo1 Stain

In order to investigate the mechanobiology of sex differences, the Piezo1 ion channel was first assessed. Beams with cells from each category were selected and fixed immediately after being stressed with the cell stressor, identical to the experiments in Aim 1. A 1:200 ratio of antibody to BSA solution was created and added onto the beams with cells post-PFA fixation. A 1:8000 DAPI ratio to PBS was added as well to stain each nucleus. Piezo1 primary and secondary antibody information can be found in Appendix P, below.

### Mitochondrial Stain

To stain and identify the mitochondria of each fibroblast, a MitoTracker from Invitrogen was used. The needed concentration when the solution is mixed was 5 μM for 30 minutes at 37° C. In order to accomplish that, 5 mg of MitoTracker was diluted in 1 mL dimethyl sulfoxide (DMSO) resulting in 20 aliquots of 50 μL MitoTracker DMSO solution. Then, calculations were made to determine how much of that solution was necessary for 10 g/mL desired concentration for mice cardiac fibroblasts [122].

$${c}_{1} \times {v}_{1} = {c}_{2} \times {v}_{2}$$

$$( 500 \frac{µg}{mL} ) ( x ) = ( 10 \frac{µg}{mL} ) ( 15 mL )$$

$$x = \frac{10 \frac{µg}{mL} \times 15 mL}{5000 \frac{µg}{mL}} = 0.03 mL$$

$$x = 30 µL$$

Once it was determined that 30 μL of the DMSO and MitoTracker solution were needed for the desired amount of media, 15 mL fibroblast media was transferred into a 15 mL conical tube. 30 μL of media was removed and 30 μL of the MitoTracker DMSO stock was added. This solution was used to submerge the control beams as well as the beams while they are in the cell stressor device for the 30 minutes in the incubator. Immediately after the stress, images were taken.

### Beam Imaging and Quantification

The beams with cells were imaged using a confocal microscope when assessing Piezo1. Quantification was performed using the ImageJ software's macro tool in which the steps for measuring the Piezo-positive area. Sample macro code is shown in Appendix P, below. Following obtaining a quantification, the value was normalized for cell count in each image individually and all data was kept on a spreadsheet.

### Statistical Analysis

All statistics were done on the raw spreadsheet data by using t-tests comparing the means of two groups to assess significant differences in the results.

### Results and Discussion for Aim 2

### Piezo1 Ion Channel

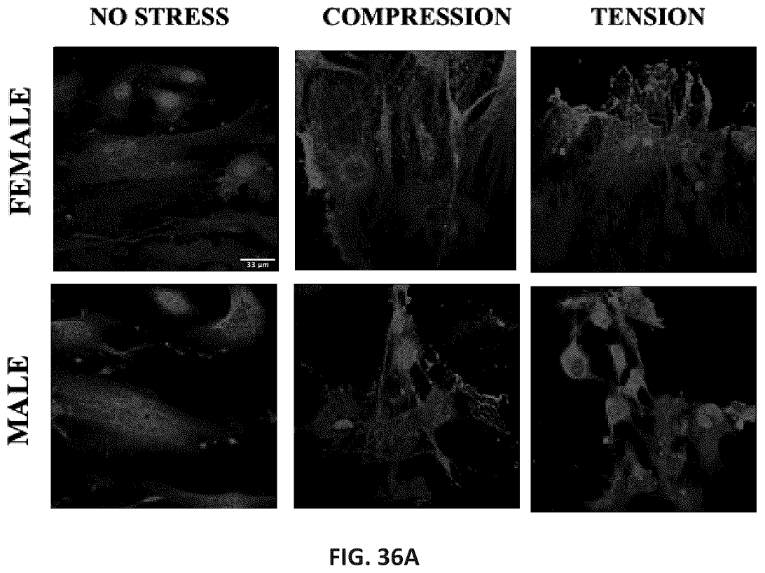

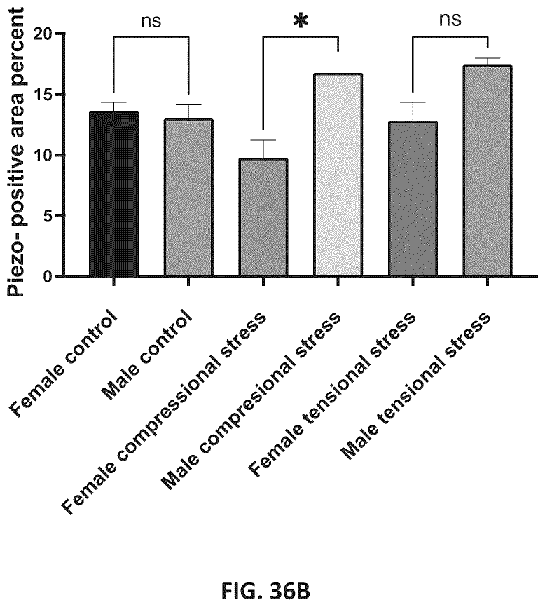

Upon staining for the Piezo1 ion channel and imaging on the confocal, it was thought that there were no sex differences. However, after quantifying the Piezo1-positive areas as a percentage per male and female controls, compressional stresses, and tensional stresses, then performing statistical analysis on those results, it can be observed that there were differences between the sexes in this ion channel after mechanical stresses were applied on the cells. There was not a statistically significant difference between the controls. However, when contrasting the female and male compressional stress, Piezo1 was significantly higher in the male fibroblast as depicted in FIG. 36A, which provides Piezo1 ion channel staining images on the confocal with scale of 33 μM. The same was observed when comparing the male and female fibroblast under tensional stress. It has been previously reported than an increase in Piezo1 could lead to fibrosis progression, as studied by Braidotti et al. who investigated this ion channel's involvement in cytoskeleton reorganization and extracellular matrix stimulation [116]. FIG. 36B demonstrates Piezo1-positive area percent versus each category for females and males (control, compression, and tension) with ‘ns’ signifying no significant difference and one * signifying a p-value≤0.05 found through a t-test with a total n=109. Since Piezo1 is highly expressed in the mitochondria, we further performed a study to evaluate the response of the mitochondria to mechanical stresses.

### Preliminary Mitochondria Results

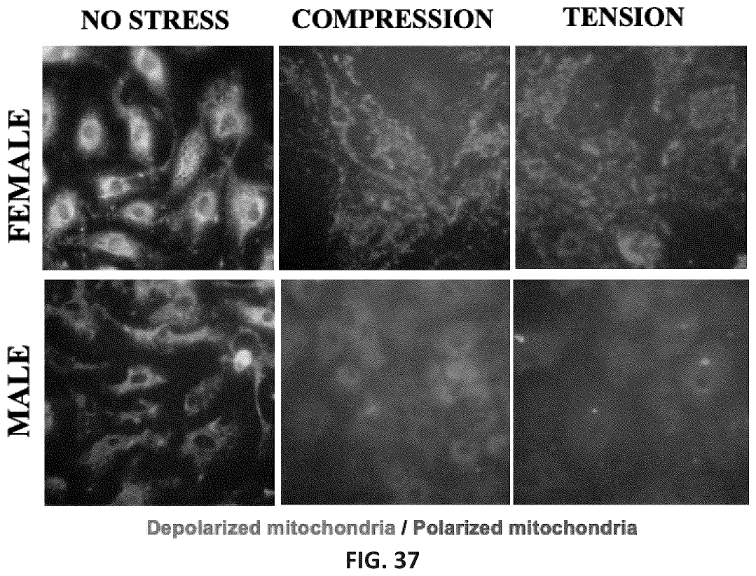

FIG. 37 depicts the microscope images taken of the female and male fibroblasts under no stress as well as compressional and tensional stresses. In FIG. 37, what is shown is mitochondria stain for females and males per stress, compression, and tension depicting depolarized mitochondria (green) and polarized mitochondria (red). The mitochondria were also investigated with the ultimate goal of linking to ion channel Piezo1. Li et al. demonstrated that Peiezo1, a mechanically sensitive ion channel, plays a crucial role in mitochondria adaptation to mechanical stimulation [123]. From these images, it can be distinguished that there are clear sex differences between the samples. The female no stress image suggests more depolarized mitochondria from its green signals than polarized mitochondria in red. Depolarized mitochondria promotes apoptotic and necrotic cell death while polarized mitochondria means the mitochondria is intact and thus sustains a highly charged membrane potential that allows for full functionality [124]. The male fibroblast mitochondria under no stress are primarily polarized. However, upon stress, the female fibroblast mitochondria transformed to be mainly polarized in both compression and tension. When under mechanical stress, the male goes from appearing polarized to having no signal at all. The green hue in the male compression and tension images are from the microscope laser being set to a higher intensity in case mitochondria could show. These preliminary results corroborate has been established before in that the females have a more favorable form of hypertrophy and recover better than their male counterparts. In regard to the mitochondria specifically, males are considered more fit and thus in the control have polarized (full functionality) mitochondria. Once under stress, the females appear to be trying to survive and that full functionality form of the mitochondria becomes activated. However, a limitation in this experiment was that the nuclei was not stained. As a result, future work would entail a MitoTracker for fixed cells as well as nuclei imaging to assess if any cells remained after stress.

### Conclusion

The long-term goal of this project was to determine sex differences in cardiac fibroblast cells under pathological mechanical stresses as well as investigating the mechanobiology with particular focus on ion channels and mitochondria function. With this aim, it was necessary to utilize a cell stressor device according to the present technology. This device provided several benefits in its simplicity, cost-efficacy, and its ability to perform both compressional and tensional stresses which is a shortcoming in other cell stressor devices on the market. Beams with each sex's fibroblast cells seeded on to it were placed into this device and stress was exerted. Therefore, an objective of this study was to assess how the fibroblasts of each sex react to mechanical stresses. Through cell staining, clear sex differences in cardiac fibroblast cells were able to be visualized in its cytoskeleton, fibroblast activation, focal adhesion/paxillin, and cell count. This visualization occurred with the confocal microscope. Intensities and area positive percentages of the appropriate marker were quantified, and statistical analysis was performed.

It was found that post-stress, while lower in count, the female fibroblast cytoskeleton maintains better form than the male. Fibroblast activation also varies between the sexes upon compressional and tensional stresses, with the male fibroblast being more activated after stress. Paxillin is crucial for heart function and its presence differed between male and female fibroblasts when stressed as well. Paxillin was lower in the male fibroblasts post compressional and tensional stress. AFM results demonstrated that female fibroblasts had a higher Young's Modulus in every category (control, compressional stress, and tensional stress) than the male fibroblasts, and thus, were stiffer.

Cell count after the stress-recovery experiments demonstrated no difference between the female control and its stresses and each of the stresses to each other. However, in the male fibroblast, differences in cell quantity were noted when comparing compression and tension, as all the cells died. When allowed to recover for 48 hours post-stress in media, female fibroblasts demonstrated the capability to recover whereas the male fibroblasts all perished and were unable to survive. After recover, paxillin presence increased in female fibroblasts after stress whereas it was almost completely diminished in male fibroblasts after stress. This indicates that our in vitro model of mechanical stresses can recapitulate an in vivo model of cardiac hypertrophy.

When examining mechanobiology, specifically ion channels and mitochondria, sex differences were observed in Piezo1 and preliminary data shows mitochondria function variation in males and females. Supplemental studies into these are necessary to further identify sex differences and begin to provide insight in to why these differences occur.

In conclusion, sex differences were observed upon compression and tensile stresses via a cell stressor device. This can be translated for other sex differences in a variety of other cell types as well for further research. Furthermore, these results can be used to engineer novel sex-specific therapeutics for HF.

### Study Limitations and Future Work

### Cell Stressor

While the current cell stressor device provides many benefits, further work could be done to motorize the cell stressor so it can mimic the heartbeat motion while the cells are stressing. This cell stressor only allows for one beam to stress at a time; thus 5 total devices were 3D printed to allow for more. However, modifications and motorization could allow for several beams to stress at a time. There could also be an inquiry into how the maximum number of use this device is able to undergo until the ABS bender deforms. Consequently, it may be useful to know how that affects how the cells react to the stresses.

### Bender

It could also prove to be helpful to improve the bender. Currently, when going to twist the screw with the toque screwdriver to exert force, the bender slips out very frequently. As a result, the bender had to be held to the body of the device carefully and tightly using tweezers until the torque screwdriver indicates it has reached 5.1 Nm. When training new students or even an experience person performing it, it is still prone to slipping out from under the device or popping out through the top. A thin shelf could be added on to the current 3D model of the stressor to prevent it from falling out the bottom. Furthermore, a plastic extension could be added to the top of the device overseeing the bender about halfway from the bender so that it does not cover the bender completely. That would also allow for the position of the bender to be observed at all times.

### Torque Screwdriver

During the course of this project, the torque screwdriver used was not capable of exerting below 5.1 Nm, which is even a very high amount of stress itself. In earlier studies, 5.2 Nm was attempted in which it killed all the cells (data not shown). Further studies could invest in a different toque screwdriver that is able to go below 5.1 Nm.

### Loss of Cells

Live and dead analysis was not able to be performed to determine loss of cells due to them sliding off the beam during media changes and during the fixing procedure. Consequently, for future experiments, live/dead could be attempted again or media-filled tissue culture plates that the beams are kept in could have their media analyzed for cells within it. These findings could help better determine, for example, if lower paxillin in males was innate or if it could be attributed to a simple loss of cell count.

### Cell Co-Cultures

While preliminary studies with NRVMs utilized a co-culture of cardiomyocytes and fibroblasts, it could be expanded to include macrophages to study its effect on fibrosis and ultimately assess its link to an ion channel. The preliminary results demonstrated that while cardiomyocytes died after stresses, they did not appear to change as notably as the fibroblast did by taking up its myofibroblast phenotype. Correspondingly, this observation was a driving force into why only fibroblast cells were examined going forward. Similarly, for the scope of this project, introducing co-cultures could have complicated the experiments by resulting in too many variables to keep track of and account for.

### Women's Health

Since the long-term goal in studying sex differences is to engineer therapies that are equally as effective in female patients to target cardiac fibrosis, it would also be worthy to investigate pregnancy, post-partum, and menopause and its effect on cardiac fibroblasts and beyond. This long duration study could lead to breakthroughs in preventing and treating peripartum cardiomyopathy, which only occurs in women during or after pregnancy [95].

### Ion Channels

While Piezo1 was investigated, further experiments can be done as well while also emphasizing TRP channels. The TRP channels are widely expressed in integral membrane proteins that are responsive to many different types of stimuli, among them mechanical stress [123, 124]. Within the heart, Piezo1 and the TRP channel family are both expressed in myocytes, fibroblast, and macrophages [127, 128]. The transient receptor potential canonical one (TRPC1), the transient receptor potential canonical six (TRPC6) and the transient receptor potential vanilloid four (TRPV4). These TRP channels can be studied because they are all expressed in both human and mouse cardiac fibroblast [128]. Moreover, TRPC1 and TRPC6 are associated with cardiac hypertrophy [129], and both TRPC6 and TRPV4 are involved in the development of cardiac fibrosis [129,130]. Although it is known that TRP channels play a role in cardiac remodeling, little is known about their specific effect on female and male fibroblast.

### Mitochondria

Further work entails repeating mitochondria experiments but with a MitoTracker that is intended for fixed cells rather than live cells, as was done in this study. This would allow the cells to be fixed in place immediately after 30 minutes of stressing rather than sitting in the Mito-Tracker media solution longer in order to image. Future work with the mitochondria could clarify and provide insight into the mechanobiology since its function ranges from metabolism to modulation of signaling events that lead to cell death or survival [131].

### Example 3

Sex differences observed in vivo in the mechanosensing and/or mechanotransduction of cells has also been demonstrated by Peter et al. [120]. On the date of electronically filing this patent application, reference [120] was freely available for reading or download via at the following website maintained by the US NIH: https://pubmed.ncbi.nlm.nih.gov/33998248/. FIG. 4A of reference [120] and the accompanying description therein provides an overview of the authors' experimental design testing isoproterenol (ISO) treatment on cardiac fibroblast (CF) activation in male and female rats during the early (3 day) or late (7 day) response. FIG. 4B of reference [120] and the accompanying description therein demonstrates myofibroblast activation and cell size of CFs after 3 days of ISO treatment measured by α-smooth muscle actin (SMA) immunostaining.

Explained by Peter et al., as quoted from reference [120]: “The fibrotic response is associated with CF proliferation but also with myofibroblast activation and the subsequent activation of profibrotic signaling. Because ISO induces CF proliferation at both early and late time points, [Peter et al.] isolated CFs at both 3 and 7 days after vehicle or ISO treatment and evaluated myofibroblast activation (FIG. 4A). Isolated CFs were cultured for 2 days in vitro and then stained for αSMA, a commonly used myofibroblast marker. Because activated myofibroblasts are larger than quiescent fibroblasts [ref. [108]], cell area was also measured. Interestingly, vehicle-treated male CFs were more activated than female CFs, as assessed by αSMA and cell area (FIG. 4B). After 3 days of ISO treatment, both male and female CFs expressed more αSMA fibers compared with vehicle controls; similarly, both male and female CFs from ISO-treated animals were larger than CFs from vehicle-treated animals. By 7 days, only ISO-treated female CFs were larger and expressed more αSMA fibers compared with vehicle-treated female CFs, whereas male vehicle treated and ISO-treated CFs were not significantly different (FIG. 4C). These data indicate that male CFs maintain higher levels of myofibroblast activation, even without ISO stimulation in vivo. Moreover, male CFs activate to myofibroblasts early, but then deactivate over 7 days of ISO treatment. Conversely, female CFs activate to myofibroblasts early and late with ISO stimulation in vivo.” (emphasis in original).

Notably, the results presented herein obtained using the cell stressor devices, systems and methods as disclosed herein provide evidence to demonstrate that in vivo results as in Peter et al.

may be replicated, or at least estimated, in vitro by practicing the present technology. Furthermore, as the disclosed cell stressor devices, systems and methods may be utilized to potentially obtain experimental results faster and less expensively as compared to in vivo work, the present technology holds great promise to accelerate basic and/or translational research, as well as drug discovery, for a variety of diseases that may involve mechanosensing and/or mechanotransduction such as heart failure, among others. Moreover, the in vivo work such as highlighted in this Example 3 [120] provides an important validation of the in vitro model enabled by the present technology.

### Example 4

The cell stressor devices, systems and methods according to the present technology may be improved by adjusting the dimensions of the beam (e.g., beam 20 of device 20, as shown and described above with references to FIGS. 8, 11A and 11B). This may involve, for any given window dimension, adjusting the dimensions of the beam, while the dimensions of window are maintained. In another example, the window dimensions may also be changed for the disclosed cell stressor device. In addition to finer control being accomplished by way of using a motorized means for turning the screw, as described above, increasing the thread count of such a screw may provide finer control.

In some cases, the use of a torque screwdriver to turn the screw of the disclosed cell stressor device to cause the bender to be moved may be replaced with a more precise method, like a stepper motor to rotate the screw. Such a more precise and accurate, and in some embodiments, automated, means of rotating the screw may be combined with modifying the dimensions of the beam and/or bender of the cell stressor device according to the present technology. In some examples, having a geared transmission configuration between a shaft of a stepper, or other type of, electric motor and the screw (e.g., screw 30 operably coupled to bender 45 as described above with references to FIGS. 8, 11A and 11B) may advantageously enable users of the disclosed cell stressor devices, systems and methods an even greater level of fine control when practicing the present technology to the various beneficial ends as discussed herein.

In a healthy heart myocardium, for example, stresses on the cells and/or tissues may be as low as 7 kPa, whereas in an injured myocardium, stresses on the cells and/or tissues may be in the range of 11-40 kPa. Using the cell stressor device according to the present technology (e.g., device 10 as described above with reference to FIGS. 8, 11A and 11B), the effective stress that may be experienced by cells cultured on the beam on account of forces acting thereupon by the bender being moved by the screw may range from 30 kPa-30 MPa.

In some embodiments according to the present technology, the lower end (e.g., 30 kPa) aforementioned range of effective stress that can be provided to cells and/or tissues cultured on the beam of the disclosed cell stressor devices may be reduced to be closer to the value (e.g., 7 kPa) of stresses experienced in healthy myocardium, and also to the lower value (e.g., 11 kPa) of the aforementioned range of stresses experienced in a injured myocardium. An example listing of ways that stresses being experienced by cells and/or tissues cultured on, or otherwise adhering to, beam may be reduced is: (a) increasing the thickness (e.g., height, denoted “HB” in FIG. 11B) of the beam; (b) applying a lower force torque by way of bender; (c) applying a general low force; and (d) any combination of (a), (b) and (c). As to item (b), a lower torque to the screw will result in a lower being applied by the bender to the beam, and thus lower stresses being experienced by the cells and/or tissues on the beam. As to item (c), decreasing the force may be enabled using an automated technique with fine motor control and possibly also a geared transmission.

Notably, applying one or more of the aforementioned techniques (a)-(d) as listed above in this Example 4 may enable a user of the disclosed system 80 to utilize, for example, a first set of one or more cell stressor devices 10 to provide a first stress level to cells and/or tissues, while a second set of one or more devices 10 may provide a second stress level to cells and/or tissues. In such a system 80, multiple replicates of control (e.g., healthy condition) sets can be combined with multiple replicates of test (e.g., diseased or injured) sets may be run in a single, or even multiple (e.g., more than one test condition, cell type, candidate treatment, patient ID, etc.) experiments under equivalently timed and controlled conditions, so as to accelerate research or diagnostic activities accomplished using the present technology.

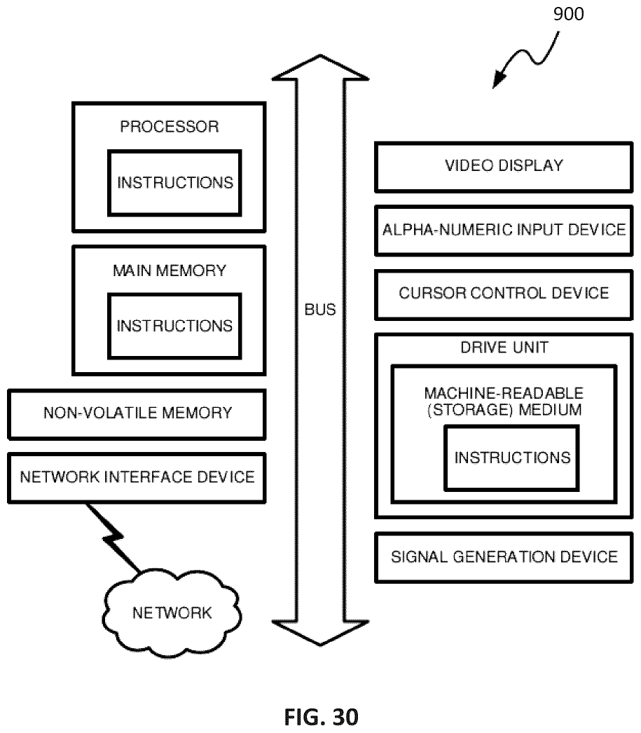

FIG. 30 is a diagrammatic representation of a machine, in the example form, of a computer system 3000 within which a set of instructions, for causing the machine to implement or otherwise perform any one or more of the techniques and methodologies of the present technology described herein, may be executed. Computer system 3000 may, for some embodiments of the present technology, be representative of controller means including, without limitation, for operating device 10 and/or system 80 according to the present technology.

In the example of FIG. 30, the computer system 3000 includes a processor, memory, non-volatile memory, and an interface device. Various common components (e.g., cache memory) are omitted for illustrative simplicity. The computer system 3000 is intended to illustrate a hardware device on which any of the components depicted in the examples of the present technology described herein (and any other components described in this specification) can be implemented. The computer system 3000 can be of any applicable known or convenient type. The components of the computer system 3000 can be coupled together via a bus or through some other known or convenient device.

The processor of computer system 3000 may be, for example, a conventional microprocessor such as an INTEL PENTIUM microprocessor or MOTOROLA POWER PC microprocessor. One of skill in the relevant art will recognize that the terms “machine-readable (storage) medium” or “computer-readable (storage) medium” include any type of device that is accessible by the processor. In some embodiments, these storage media are embodied in non-transitory computer-readable media that can store program instructions (e.g., as software or firmware) which, when executed by one or more processors of the disclosed technology, cause the controller means (e.g., a processor coupled to a motor controller) to implement, execute, or otherwise facilitate performance of the various algorithms, processes and methods disclosed herein.

In computer system 3000, the memory is coupled to the processor by, for example, a bus. The memory can include, by way of example but not limitation, random access memory (RAM), such as dynamic RAM (DRAM) and static RAM (SRAM). The memory can be local, remote, or distributed.

The bus of computer system 3000 also couples the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer system 3000. The non-volatile storage can be local, remote, or distributed. The non-volatile memory is optional because systems can be created with all applicable data available in memory. An embodiment of computer system 3000 will usually include at least a processor, memory, and a device (e.g., a bus) coupling the memory to the processor.

Software or firmware utilized by computer system 3000 may be stored in the non-volatile memory and/or the drive unit. Indeed, for large programs, it may not even be possible to store the entire program in the memory. Nevertheless, it should be understood that for software and/or firmware to run, if necessary, it is moved to a computer readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, firmware or a software program is assumed to be stored at any known or convenient location (from non-volatile storage to hardware registers) when the software program is referred to as “implemented in a computer-readable medium”. A processor is considered to be “configured to execute a program” when at least one value associated with the program is stored in a register readable by the processor.

The bus also couples the processor to the network interface device of computer system 3000. The interface can include one or more of a modem or network interface. It will be appreciated that a modem or network interface can be considered to be part of the computer system. The interface can include an analog modem, ISDN modem, cable modem, token ring interface, satellite transmission interface (e.g., “direct PC”), or other interfaces for coupling a computer system (e.g., 3000) to other computer systems. The interface can include one or more input and/or output (I/O) devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device. For simplicity, it is assumed that controllers of any devices not depicted in the example of FIG. 30 reside in the interface.

In operation, the computer system 3000 can be controlled by operating system software that includes a file management system, such as a disk operating system. One example of operating system software with associated file management system software is the family of operating systems known as WINDOWS from MICROSOFT Corporation of Redmond, Washington, and their associated file management systems. Another example of operating system software with its associated file management system software is the LINUX operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The algorithms and displays presented herein are not inherently related to any particular computer or other apparatus. Various general-purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may thus be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an IPHONE, a BLACKBERRY, a processor, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above detailed description using the singular or plural number may also include the plural or singular number, respectively. The word “or,” in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are, at times, shown as being performed in a series, these processes or blocks may instead be performed in parallel, or may be performed at different times. Further, any specific numbers noted herein are only examples: alternative implementations may employ differing values or ranges.

The teachings of the disclosure provided herein can be applied to other systems, not necessarily the system described above. For instance, the present technology may be beneficially applied by persons having ordinary skill in the art in applications other than strictly for cardiac cells and tissues where characterizing biomechanical and/or biophysical effects is useful. Likewise, the elements and acts of the various embodiments described above can be combined to provide further embodiments.

Any patents or patent applications and other references noted herein, including as listed in the section entitled REFERENCES, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above detailed description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above detailed description section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112(f), other aspects may likewise be embodied as a means-plus-function claim, or in other forms, such as being embodied in a computer-readable medium. (Any claims intended to be treated under 35 U.S.C. § 112(f) will begin with the words “means for”.) Accordingly, the applicant reserves the right to add additional claims after filing the application to pursue such additional claim forms for other aspects of the disclosure.

The detailed description provided herein may be applied to other systems, not necessarily only the system described above. The elements and acts of the various examples described above can be combined to provide further implementations of the invention. Some alternative implementations of the invention may include not only additional elements to those implementations noted above, but also may include fewer elements. These and other changes can be made to the invention in light of the above detailed description. While the above description defines certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above detailed description section explicitly defines such terms. Accordingly, the actual scope of the invention encompasses not only the disclosed examples, but also all equivalent ways of practicing or implementing the invention.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Moreover, although specific embodiments have been illustrated and described herein, it should be appreciated that any subsequent arrangement designed to achieve the same or similar purpose may be substituted for the specific embodiments shown.

This disclosure is intended to cover any and all subsequent adaptations or variations of various embodiments. Combinations of the above embodiments can be made, and other embodiments not specifically described herein will be apparent to those of skill in the art upon reviewing the description. Additionally, the illustrations are merely representational and may not be drawn to scale. Certain proportions within the illustrations may be exaggerated, while other proportions may be reduced. Accordingly, the disclosure and the figures are to be regarded as illustrative and not restrictive.

### REFERENCES

1. Friedrich, O., Wagner, S., Battle, A. R., Schurmann, S. & Martinac, B. Mechano-regulation of the beating heart at the cellular level—Mechanosensitive channels in normal and diseased heart. Prog. Biophys. Mol. Biol. 110, 226-238 (2012).
2. Teng, J., Loukin, S. & Kung, C. Mechanosensitive Ion Channels in Cardiovascular Physiology. Exp. Clin. Cardiol. 20, 6550-6560 (2016).
3. Jiang, F. et al. The mechanosensitive Piezo1 channel mediates heart mechano-chemo transduction. Nat. Commun. 12, 1-14 (2021).
4. AP, V. & HC, H. Biomechanics of Cardiac Function Andrew. Compr Physiol. 5, 1623-44 (2016).
5. Ziaeian, B. & Fonarow, G. C. Epidemiology and aetiology of heart failure. Nat. Rev. Cardiol. 13, 368-378 (2016).
6. Aparicio, H. J. et al. Heart Disease and Stroke Statistics—2021 Update A Report from the American Heart Association. Circulation (2021). doi:10.1161/CIR.0000000000000950.
7. Heidenreich, P. A. et al. Forecasting the impact of heart failure in the united states a policy statement from the american heart association. Circ. Hear. Fail. 6, 606-619 (2013).
8. Urbich, M. et al. A Systematic Review of Medical Costs Associated with Heart Failure in the USA (2014-2020). Pharmacoeconomics 38, 1219-1236 (2020).
9. Monteiro, L. M., Vasques-Nóvoa, F., Ferreira, L., Pinto-do-O, P. & Nascimento, D. S. Restoring heart function and electrical integrity: closing the circuit. npj Regen. Med. 2, 9 (2017).
10. Bytyçi, I. & Bajraktari, G. Mortality in heart failure patients. Anadolu Kardiyol. Derg. 15, 63-68 (2015).
11. Taylor, C. J. et al. Trends in survival after a diagnosis of heart failure in the United Kingdom 2000-2017: population based cohort study. BMJ 364, 1-10 (2019).
12. Garbade, J., Barten, M. J., Bittner, H. B. & Mohr, F.-W. Heart transplantation and left ventricular assist device therapy: two comparable options in end-stage heart failure? Clin. Cardiol. 36, 378-82 (2013).
13. Macdonald, P., Verran, D., O'Leary, M., Cavazzoni, E. & Dhital, K. Heart transplantation from donation after circulatory death donors. Transplantation 99, 1101-1102 (2015).
14. Travers, J. G., Kamal, F. A., Robbins, J., Yutzey, K. E. & Blaxall, B. C. Cardiac fibrosis: The fibroblast awakens. Circ. Res. 118, 1021-1040 (2016).
15. Aplin, A. E. et al. Integrin-Mediated Adhesion Regulates ERK Nuclear Translocation and Phosphorylation of Stable URL: http://www.jstor.org/stable/1620446 REFERENCES Linked references are available on JSTOR for this article: Integrin-mediated Adhesion Regulates ERK Nuclear. 153, 273-281 (2016).
16. Pazos-López, P. et al. The causes, consequences, and treatment of left or right heart failure. Vasc. Health Risk Manag. 7, 237-254 (2011).
17. Braunwald, E. Heart failure. JACC Hear. Fail. 1, 1-20 (2013).
18. Sergeeva, I. A. & Christoffels, V. M. Regulation of expression of atrial and brain natriuretic peptide, biomarkers for heart development and disease. Biochim. Biophys. Acta—Mol. Basis Dis. 1832, 2403-2413 (2013).
19. Ikegami, R., Shimizu, I., Yoshida, Y. & Minamino, T. Metabolomic Analysis in Heart Failure. Circ. J. 82, 10-16 (2018).
20. Litvinukovi, M. et al. Cells of the adult human heart. Nature 588, 466-472 (2020).
21. Howard, C. M. & Baudino, T. A. Dynamic cell-cell and cell-ECM interactions in the heart. J. Mol. Cell. Cardiol. 70, 19-26 (2014).
22. Deb, A. Cell-cell interaction in the heart via Wnt/0-catenin pathway after cardiac injury. Cardiovasc. Res. 102, 214-223 (2014).
23. Talman, V. & Ruskoaho, H. Cardiac fibrosis in myocardial infarction—from repair and remodeling to regeneration. Cell Tissue Res. 365, 563-581 (2016).
24. Hinz, B. Myofibroblasts. Exp Eye Res 142, 56-70 (2016).
25. Francis Stuart, S. D., De Jesus, N. M., Lindsey, M. L. & Ripplinger, C. M. The crossroads of inflammation, fibrosis, and arrhythmia following myocardial infarction. Journal of Molecular and Cellular Cardiology vol. 91 114-122 (2016).
26. Furtado, M. B., Costa, M. W. & Rosenthal, N. A. The cardiac fibroblast: Origin, identity and role in homeostasis and disease. Differentiation vol. 92 93-101 (2016).
27. Davis, J. & Molkentin, J. D. Myofibroblasts: Trust your heart and let fate decide. Journal of Molecular and Cellular Cardiology vol. 70 9-18 (2014).
28. Richardson, W. J., Clarke, S. A., Quinn, T. A. & Holmes, J. W. Physiological Implications of Myocardial Scar Structure. Compr. Physiol. 5, 1877-1909 (2015).
29. Kong, P., Christia, P. & Frangogiannis, N. G. The pathogenesis of cardiac fibrosis. Cellular and Molecular Life Sciences vol. 71 549-574 (2014).
30. Krenning, G., Zeisberg, E. M. & Kalluri, R. The origin of fibroblasts and mechanism of cardiac fibrosis. Journal of Cellular Physiology vol. 225 631-637 (2010).
31. Buyandelger, B., Mansfield, C. & Knöll, R. Mechano-signaling in heart failure. Pflügers Arch.—Eur. J. Physiol. 466, 1093-1099 (2014).
32. Emig, R. et al. Piezo1 Channels Contribute to the Regulation of Human Atrial Fibroblast Mechanical Properties and Matrix Stiffness Sensing. Cells 10, 1-20 (2021).
33. Fu, X. et al. Specialized fibroblast differentiated states underlie scar formation in the infarcted mouse heart. J. Clin. Invest. 128, 2127-2143 (2018).
34. Mak, M., Spill, F., Kamm, R. D. & Zaman, M. H. Single-cell migration in complex microenvironments: Mechanics and signaling dynamics. J. Biomech. Eng. 138, (2016).
35. Drews, G. Contributions of Theodor Wilhelm Engelmann on phototaxis, chemotaxis, and photosynthesis. Photosynth Res 83, 25-34 (2005).
36. Sunyer, R. & Trepat, X. Durotaxis. Curr. Biol. 30, R383-R387 (2020).
37. Lo, C. M., Wang, H. B., Dembo, M. & Wang, Y. L. Cell movement is guided by the rigidity of the substrate. Biophys. J. 79, 144-152 (2000).
38. Shellard, A. & Mayor, R. All Roads Lead to Directional Cell Migration. Trends Cell Biol. 30, 852-868 (2020).
39. Lele, T. P., Brock, A. & Peyton, S. R. Emerging Concepts and Tools in Cell Mechanomemory. Ann. Biomed. Eng. 48, 2103-2112 (2020).
40. Gaetani, R. et al. When Stiffness Matters: Mechanosensing in Heart Development and Disease. Front. Cell Dev. Biol. 8, 1-16 (2020).
41. Sirry, M. S. et al. Characterisation of the mechanical properties of infarcted myocardium in the rat under biaxial tension and uniaxial compression. J. Mech. Behav. Biomed. Mater. 63, 252-264 (2016).
42. Todaro, G. J. & Green, H. Quantitative studies of the growth of mouse embryo cells in culture and their development into established lines. J Cell Biol 17, 299-313 (1963).
43. Busin, L., Vandenbroucke, N. & Macaire, L. Color spaces and image segmentation. Adv. imaging electron Phys. 151, 1 (2008).
44. Deter, H. S., Dies, M., Cameron, C. C., Butzin, N. C. & Buceta, J. A Cell Segmentation/Tracking Tool Based on Machine Learning. Methods Mol Biol 2040, 399-422 (2019).
45. Scherr, T., Löffler, K., Böhland, M. & Mikut, R. Cell segmentation and tracking using CNN-based distance predictions and a graph-based matching strategy. PLoS One 15, e0243219 (2020).
46. Stilgoe, J. Machine learning, social learning and the governance of self-driving cars. Soc. Stud. Sci. 48, 25-56 (2017).
47. Shrivakshan, G. T. & Chandrasekar, C. A comparison of various edge detection techniques used in image processing. Int. J. Comput. Sci. Issues 9, 269 (2012).
48. Ganesan, P. & Sajiv, G. A comprehensive study of edge detection for image processing applications. in 2017 International Conference on Innovations in Information, Embedded and Communication Systems (ICIIECS) 1-6 (2017). doi:10.1109/ICIIECS.2017.8275968.
49. Meijering, E., Dzyubachyk, O. & Smal, I. Methods for cell and particle tracking. Methods Enzym. 504, 183-200 (2012).
50. Borst, A. & Egelhaaf, M. Principles of visual motion detection. Trends Neurosci. 12, 297-306 (1989).
51. Frykman, P. & Rogon, T. A. Anisotropy in pore networks analyzed with 2-D autocorrelation (variomaps). Comput. Geosci. 19, 887-930 (1993).
52. Springer, R., Zielinski, A., Pleschka, C., Hoffmann, B. & Merkel, R. Unbiased pattern analysis reveals highly diverse responses of cytoskeletal systems to cyclic straining. PLoS One 14, e0210570 (2019).
53. Bennig, G. K. Atomic force microscope and method for imaging surfaces with atomic resolution. (1988).
54. Binnig, G., Quate, C. F. & Gerber, C. Atomic force microscope. Phys Rev Lett 56, 930-933 (1986).
55. Allison, D. P., Mortensen, N. P., Sullivan, C. J. & Doktycz, M. J. Atomic force microscopy of biological samples. Wiley Interdisciplinary Reviews: Nanomedicine and Nanobiotechnology vol. 2 618-634 (2010).
56. Horobin, R. W. Biological staining: mechanisms and theory. Biotech. Histochem. 77, 3-13 (2002).
57. Decherchi, P., Cochard, P. & Gauthier, P. Dual staining assessment of Schwann cell viability within whole peripheral nerves using calcein-AM and ethidium homodimer. J. Neurosci. Methods 71, 205-213 (1997).
58. MINSKY, M. Microscopy apparatus. (1961).
59. Nwaneshiudu, A. et al. Introduction to Confocal Microscopy. J. Invest. Dermatol. 132, 1-5 (2012).
60. Elliott, A. D. Confocal Microscopy: Principles and Modern Practices. Curr. Protoc. Cytom. 92, e68-e68 (2020).
61. ASTM E855-08 Standard Test Methods for Bend Testing of Metallic Flat Materials for Spring Applications Involving Static Loading. https://www.astm.org/DATABASE.CART/HISTORICAL/E855-08.htm.
62. Niblack, W. An introduction to digital image processing. (Strandberg Publishing Company, 1985).
63. Fabrizio, J., Marcotegui, B. & Cord, M. Text segmentation in natural scenes using Toggle-Mapping. in 2009 16th IEEE International Conference on Image Processing (ICIP) 2373-2376 (2009). doi:10.1109/ICIP.2009.5413435.
64. Liu, B. et al. Role of cyclic strain frequency in regulating the alignment of vascular smooth muscle cells in vitro. Biophys. J. 94, 1497-1507 (2008).
65. Mousavizadeh, R. et al. Cyclic strain alters the expression and release of angiogenic factors by human tendon cells. PLoS One 9, e97356 (2014).
66. Colombo, A., Guha, S., Mackle, J. N., Cahill, P. A. & Lally, C. Cyclic strain amplitude dictates the growth response of vascular smooth muscle cells in vitro: role in in-stent restenosis and inhibition with a sirolimus drug-eluting stent. Biomech. Model. Mechanobiol. 12, 671-683 (2013).
67. Qi, Y.-X. et al. Cyclic strain modulates migration and proliferation of vascular smooth muscle cells via Rho-GDIalpha, Rac1, and p38 pathway. J. Cell. Biochem. 109, 906-914 (2010).
68. Metzler, S. A., Pregonero, C. A., Butcher, J. T., Burgess, S. C. & Warnock, J. N. Cyclic strain regulates pro-inflammatory protein expression in porcine aortic valve endothelial cells. J. Heart Valve Dis. 17, 571-7; discussion 578 (2008).
69. Duszyc, K. et al. Mechanotransduction activates RhoA in the neighbors of apoptotic epithelial cells to engage apical extrusion. Curr. Biol. 31, 1326-1336 (2021).
70. Tarbell, J. M., Weinbaum, S. & Kamm, R. D. Cellular fluid mechanics and mechanotransduction. Ann. Biomed. Eng. 33, 1719-1723 (2005).
71. Quinn, T. A. & Kohl, P. Rabbit models of cardiac mechano-electric and mechano-mechanical coupling. Progress in Biophysics and Molecular Biology vol. 121 110-122 (2016).
72. Abramochkin, D. V., Lozinsky, I. T. & Kamkin, A. Influence of mechanical stress on fibroblast-myocyte interactions in mammalian heart. Journal of Molecular and Cellular Cardiology vol. 70 27-36 (2014).
73. Friedrich, O., Wagner, S., Battle, A. R., Schurmann, S. & Martinac, B. Mechano-regulation of the beating heart at the cellular level—Mechanosensitive channels in normal and diseased heart. Prog Biophys Mol Biol 110, 226-238 (2012).
74. Teng, J., Loukin, S. & Kung, C. Mechanosensitive Ion Channels in Cardiovascular Physiology. Exp Clin Cardiol 20, 6550-6560 (2014).
75. Voorhees, A. P. & Han, H.-C. Biomechanics of Cardiac Function. Compr Physiol 5, 1623-44 (2015).
76. Jiang, F. et al. The mechanosensitive Piezo1 channel mediates heart mechano-chemo transduction. Nat Commun 12, 869 (2021).
77. st. Pierre, S. R., Peirlinck, M. & Kuhl, E. Sex Matters: A Comprehensive Comparison of Female and Male Hearts. Frontiers in Physiology vol. 13 Preprint at https://doi.org/10.3389/fphys.2022.831179 (2022).
78. Komuro, I. Molecular mechanism of mechanical stress-induced cardiac hypertrophy. Jpn Heart J 41, 117-29 (2000).
79. Prabhavathi, K., Selvi, K. T., Poornima, K. N. & Sarvanan, A. Role of biological sex in normal cardiac function and in its disease outcome—a review. J Clin Diagn Res 8, BE01-4 (2014).
80. Prajapati, C., Koivumaki, J., Pekkanen-Mattila, M. & Aalto-Setal, K. Sex differences in heart: from basics to clinics. Eur J Med Res 27, 241 (2022).
81. Beale, A. L., Meyer, P., Marwick, T. H., Lam, C. S. P. & Kaye, D. M. Sex Differences in Cardiovascular Pathophysiology. Circulation 138, 198-205 (2018).
82. Lam, C. S. P. et al. Sex differences in heart failure. Eur Heart J 40, 3859-3868c (2019).
83. Heidenreich, P. A. et al. Forecasting the Impact of Heart Failure in the United States. Circ Heart Fail 6, 606-619 (2013).
84. Heidenreich, P. A. et al. Forecasting the impact of heart failure in the United States a policy statement from the American Heart Association. Circ Heart Fail 6, 606-619 (2013).
85. Urbich, M. et al. A Systematic Review of Medical Costs Associated with Heart Failure in the USA (2014-2020). Pharmacoeconomics 38, 1219-1236 (2020).
86. Komanduri, S., Jadhao, Y., Guduru, S. S., Cheriyath, P. & Wert, Y. Prevalence and risk factors of heart failure in the USA: NHANES 2013-2014 epidemiological follow-up study. J Community Hosp Intern Med Perspect 7, 15-20 (2017).
87. Virani, S. S. et al. Heart Disease and Stroke Statistics—2021 Update. Circulation 143, (2021).
88. Halliday, B. P. et al. Sex- and age-based differences in the natural history and outcome of dilated cardiomyopathy. Eur J Heart Fail 20, 1392-1400 (2018).
89. Lala, A. et al. Sex Differences in Heart Failure. J Card Fail 28, 477-498 (2022).
90. Hsich, E. M. Sex Differences in Advanced Heart Failure Therapies. Circulation 139, 1080-1093 (2019).
91. Tripathi, R. et al. Enhanced heart failure, mortality and renin activation in female mice with experimental dilated cardiomyopathy. PLoS One 12, e0189315 (2017).
92. Gattazzo, F., Urciuolo, A. & Bonaldo, P. Extracellular matrix: A dynamic microenvironment for stem cell niche. Biochimica et Biophysica Acta (BBA)—General Subjects 1840, 2506-2519 (2014).
93. Beale, A. L., Meyer, P., Marwick, T. H., Lam, C. S. P. & Kaye, D. M. Sex Differences in Cardiovascular Pathophysiology: Why Women Are Overrepresented in Heart Failure With Preserved Ejection Fraction. Circulation 138, 198-205 (2018).
94. Wu, J., Dai, F., Li, C. & Zou, Y. Gender Differences in Cardiac Hypertrophy. J Cardiovasc Transl Res 13, 73-84 (2020).
95. Regitz-Zagrosek, V. Sex and Gender Differences in Heart Failure. International Journal of Heart Failure 2, 157 (2020).
96. Garcia, M., Mulvagh, S. L., Bairey Merz, C. N., Buring, J. E. & Manson, J. E. Cardiovascular Disease in Women. Circ Res 118, 1273-1293 (2016).
97. Wynn, T. A. & Ramalingam, T. R. Mechanisms of fibrosis: therapeutic translation for fibrotic disease. Nat Med 18, 1028-1040 (2012).
98. Medzikovic, L., Aryan, L. & Eghbali, M. Connecting sex differences, estrogen signaling, and microRNAs in cardiac fibrosis. J Mol Med 97, 1385-1398 (2019).
99. Humeres, C. & Frangogiannis, N. G. Fibroblasts in the Infarcted, Remodeling, and Failing Heart. JACC Basic Transl Sci 4, 449-467 (2019).
100. Travers, J. G., Kamal, F. A., Robbins, J., Yutzey, K. E. & Blaxall, B. C. Cardiac Fibrosis. Circ Res 118, 1021-1040 (2016).
101. Gibb, A. A., Lazaropoulos, M. P. & Elrod, J. W. Myofibroblasts and Fibrosis. Circ Res 127, 427-447 (2020).
102. Chen, Y., Ju, L., Rushdi, M., Ge, C. & Zhu, C. Receptor-mediated cell mechanosensing. Mol Biol Cell 28, 3134-3155 (2017).
103. Meagher, P. B. et al. Cardiac Fibrosis: Key Role of Integrins in Cardiac Homeostasis and Remodeling. Cells 10, 770 (2021).
104. Goldmann, W. H. Mechanosensation: A basic cellular process. in Progress in Molecular Biology and Translational Science vol. 126 75-102 (Elsevier B.V., 2014).
105. De, R. Cell Mechanosensing. Resonance 24, 289-296 (2019).
106. Wynn, T. A. & Ramalingam, T. R. Mechanisms of fibrosis: Therapeutic translation for fibrotic disease. Nature Medicine vol. 18 1028-1040 Preprint at https://doi.org/10.1038/nm.2807 (2012).
107. Chen, X., O'Connell, T. D. & Xiang, Y. K. With or Without Langendorff. Circ Res 119, 888-890 (2016).
108. Baum, J. & Duffy, H. S. Fibroblasts and Myofibroblasts: What Are We Talking About? J Cardiovasc Pharmacol 57, 376-379 (2011).
109. Akiyama, S. K., Yamada, K. M. & Hayashi, M. The structure of fibronectin and its role in cellular adhesion. J Supramol Struct Cell Biochem 16, 345-358 (1981).
110. Extracellular Matrix Molecules. in Cell Biology 505-524 (Elsevier, 2017). doi:10.1016/B978-0-323-34126-4.00029-3.
111. Kihm, K. D. Confocal Laser Scanning Microscopy (CLSM). in Near-Field Characterization of Micro/Nano-Scaled Fluid Flows 55-79 (Springer Berlin Heidelberg, 2011). doi:10.1007/978-3-642-20426-5_4.
112. Elliott, A. D. Confocal Microscopy: Principles and Modern Practices. Curr Protoc Cytom 92, e68 (2020).
113. Vahabi, S., Nazemi Salman, B. & Javanmard, A. Atomic force microscopy application in biological research: a review study. Iran J Med Sci 38, 76-83 (2013).
114. Pena, B., Adbel-Hafiz, M., Cavasin, M., Mestroni, L. & Sbaizero, O. Atomic Force Microscopy (AFM) Applications in Arrhythmogenic Cardiomyopathy. Int J Mol Sci 23, (2022).
115. Maity, B., Sheff, D. & Fisher, R. A. Immunostaining. in 81-105 (2013). doi:10.1016/B978-0-12-407239-8.00005-7.
116. Braidotti, N., Chen, S. N., Long, C. S., Cojoc, D. & Sbaizero, O. Piezo1 Channel as a Potential Target for Hindering Cardiac Fibrotic Remodeling. Int J Mol Sci 23, 8065 (2022).
117. Garvin, A. M. & Hale, T. M. State of change: epigenetic and mitochondrial regulation of cardiac fibroblast activation. Curr Opin Physiol 28, 100557 (2022).
118. Shinde, A. V., Humeres, C. & Frangogiannis, N. G. The role of α-smooth muscle actin in fibroblast-mediated matrix contraction and remodeling. Biochimica et Biophysica Acta (BBA)—Molecular Basis of Disease 1863, 298-309 (2017).
119. Vasquez, C., Benamer, N. & Morley, G. E. The Cardiac Fibroblast: Functional and Electrophysiological Considerations in Healthy and Diseased Hearts. J Cardiovasc Pharmacol 57, 380-388 (2011).
120. Peter, A. K. et al. Cardiac Fibroblasts Mediate a Sexually Dimorphic Fibrotic Response to β-Adrenergic Stimulation. J Am Heart Assoc 10, e018876 (2021).
121. Chowdhury, D. et al. A proteomic view of isoproterenol induced cardiac hypertrophy: prohibitin identified as a potential biomarker in rats. J Transl Med 11, 130 (2013).
122. JC-1 and JC-9 Mitochondrial Potential Sensors. (2006).
123. Li, X., Kordsmeier, J., Nookaew, I., Kim, H. & Xiong, J. Piezo1 stimulates mitochondrial function via <scp>cAMP</scp> signaling. The FASEB Journal 36, (2022).
124. Walsh, D. W. M. et al. Live cell imaging of mitochondria following targeted irradiation in situ reveals rapid and highly localized loss of membrane potential. Sci Rep 7, 46684 (2017).
125. Zheng, J. Molecular Mechanism of TRP Channels. in Comprehensive Physiology 221-242 (Wiley, 2013). doi:10.1002/cphy.c120001.
126. Samanta, A., Hughes, T. E. T. & Moiseenkova-Bell, V. Y. Transient Receptor Potential (TRP) Channels. in 141-165 (2018). doi:10.1007/978-981-10-7757-9_6.
127. Stewart, L. & Turner, N. A. Channeling the Force to Reprogram the Matrix: Mechanosensitive Ion Channels in Cardiac Fibroblasts. Cells 10, 990 (2021).
128. Yue, Z., Zhang, Y., Xie, J., Jiang, J. & Yue, L. Transient Receptor Potential (TRP) Channels and Cardiac Fibrosis. Curr Top Med Chem 13, 270-282 (2013).
129. Wen, H., Gwathmey, J. K. & Xie, L.-H. Role of Transient Receptor Potential Canonical Channels in Heart Physiology and Pathophysiology. Front Cardiovasc Med 7, 24 (2020).
130. Adapala, R. K. et al. TRPV4 Mechanotransduction in Fibrosis. Cells 10, 3053 (2021).
131. Urbani, A., Prosdocimi, E., Carrer, A., Checchetto, V. & Szabò, I. Mitochondrial Ion Channels of the Inner Membrane and Their Regulation in Cell Death Signaling. Front Cell Dev Biol 8, (2021).
132. Yang, D. et al. A new method for neonatal rat ventricular myocyte purification using superparamagnetic iron oxide particles. Int J Cardiol 270, 293-301 (2018).
133. Peña, B. et al. Cellular Biomechanic Impairment in Cardiomyocytes Carrying the Progeria Mutation: An Atomic Force Microscopy Investigation. Langmuir 38, 14928-14940 (2022).

### APPENDICES

### Appendix A. AFM Methods and Preliminary Results

Appendix A provides additional promising preliminary results.

### AFM Analysis

After the 30 minutes of stress, the beams were adhered to a glass slide using superglue glue. 100 microliters of media was then added to the surface of the beam in order to cover just the cells. An AFM (JPK NanoWizard 4a), in force spectroscopy mode, was used assess the mechanical properties of cells. MLCT AFM probes were used, these have a pyramidal tip made of Si3N4, with a curvature radius of 35°, manufactured by Bruker. The cantilever spring constants were systematically measured using the thermal tune method and has been found to be in the range of 0.01 to 0.02 N/m. The maximal force applied to the cell has been limited to 1.5 nN in order to preserve the membrane integrity and with 5 μm of Z piezo. All the physical and physiological cues regarding AFM analysis were kept fixed. The force-distance curves were then analyzed using the Hertz model (art. Model de Hertz) to obtain the elasticity of cells studied using the JPK data processing software.

The stiffness of the cells was measured using the AFM technique described above. It was seen that the relative young's modulus of the stressed sample was significantly higher than that of the control sample.

FIG. 15. AFM results show significant differences between the stiffness of the stressed sample and the control.

### Appendix B. Four-Point Bend Stress Equation Derivation

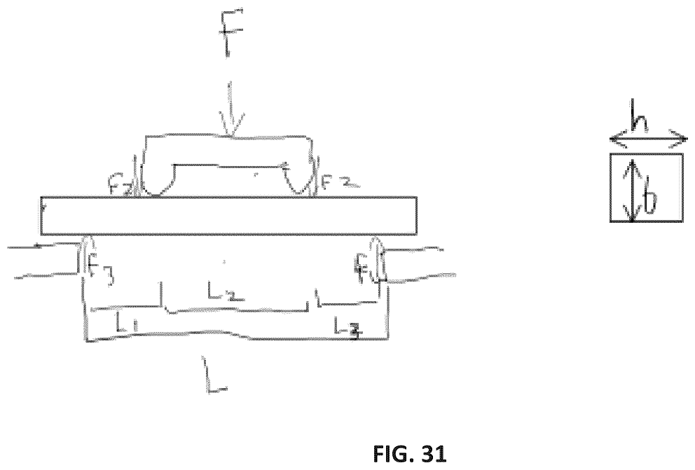

FIG. 31 illustrates a cell stressor device according to embodiments of the present technology.

Equations from Geometry and Beam Shape

$${L}_{2} = \frac{2}{3} L$$

$${L}_{1} + {L}_{2} + {L}_{3} = L$$

$${L}_{1} = \frac{1}{6} L = {L}_{3}$$

$${I}_{r e g} = \frac{b {h}^{3}}{12}$$

$$σ = \frac{M y}{I}$$

### Using Momentum to Solve for F4

$$\sum M = 0$$

$$Σ {M}_{@ F 3} = - {F}_{2} \cdot {L}_{{1}^{†}} - {F}_{2} \cdot ( {L}_{1} + {L}_{2} ) + {F}_{4} \cdot ( {L}_{1} + {L}_{2} + {L}_{3} ) = 0$$

$${F}_{4} = \frac{{F}_{2} ( 2 {L}_{1} t {L}_{2} )}{{L}_{1} + {L}_{2} + {L}_{3}}$$

### Right Half Moment

$${M}_{C e n t} = - {F}_{2} \cdot \frac{{L}_{2}}{2} + {F}_{4} \cdot ( \frac{{L}_{2}}{2} + {L}_{3} )$$

### Center to Bend Surface Distance

$$y = \frac{h}{2}$$

Plugging Moment, Inertia and Distance into Equation

$$σ = \frac{( - {F}_{2} \frac{{L}_{2}}{2} + {F}_{4} \cdot \frac{{L}_{2}}{2} + {L}_{3} ) \cdot \frac{h}{2}}{\frac{1}{12} b {h}^{3}}$$

### Plugging in F4

$$σ = \frac{( ( - {F}_{2} \cdot \frac{{L}_{2}}{2} ) + ( \frac{{F}_{2} ( 2 {L}_{1} + {L}_{2} )}{{L}_{1} + {L}_{2} + {L}_{3}} \cdot ( \frac{{L}_{2}}{2} + {L}_{3} ) ) ) \cdot \frac{1}{2}}{\frac{1}{12} b {h}^{2}}$$

Replacing all Length Using the Assumptions from the Geometry

$$σ = \frac{- {F}_{2} \cdot \frac{1}{3} L + ( \frac{{F}_{2} L}{L} ) \cdot \frac{1}{3} L + \frac{1}{6} L \cdot \frac{1}{2}}{\frac{1}{12} {bh}^{2}}$$

### Simplifying

$$σ = \frac{FL}{2 {bh}^{2}}$$

### Appendix C. Variomap Test Images and Example

The 2D-autocorelation method was applied to test images having vertical, horizontal, or angled lines. The resulting variomaps' center summarized the orientation of the lines in the binary image. This can be seen in FIG. 29 where the variomap of the vertical lines has the major axis pointing vertically, the variomap of the horizontal lines has the major axis pointing horizontally and the variomap of the angled lines had the major axis pointing at the similar angle as the lines.

FIG. 16. Examples of variomaps generated from sample binary images. First row shows lines that are perfectly vertical. The resulting variomap's center shows a vertical orientation. Second row shows lines that are perfectly horizontal. The resulting variomap's center shows a horizontal orientation. Last row shows lines that are angled. The resulting variomap's center shows the same angle as the lines in the binary image.

### Appendix D. MATLAB Stitching Script

```
%% Starting with a clean workspace
clc; clear all; close all;% makes sure that you are starting with a clean workspace
%% Setting the path that you look for photos in
pat(1)=“/Volumes/USB DISK/24hex921/c”; % Set the paths that you want the code to cycle
through. Here you can add as many or as few as you want.
%% Main loop to cycle throught all your folders and find pictures
for dnu=1:length(pat) % This loop is used to cycle through all the paths that you set in the
previous section
  path=pat(dnu);% This is where you are setting the current path that you look through
  oldpath=cd (path);% This changes the current folder to the one set in the path. this is manily
used so that the stitched image can be saved to the same file
  readPath=dir(fullfile(path,‘**/*.tif’));% this is looking for images of the file type .tif if you are
using different file type you can change the file type
  for i=1:length(readPath) % this loops through the entire read path to read all the
    image(i).o=imread(fullfile(readPath(i).folder,readPath(i).name));
    image(i).d= (im2gray(image(i).o));
    figure;imshow(image(i).o);
  end
  Fimg=image(1).d;
  for ni=2:length(readPath)
    F=Fimg;
    S=image(ni).d;
    [rows cols] = size(F);
    [rows2 cols2] = size(S);
    Tmp = [ ];
    S1 = [ ];
    S1= S(1:20,:);
    for k = 1:rows−20% to prevent k to go beyond boundaries.
      F1(1:20,:) = F(k:k+19,:);
      temp= corr2(F1,S1);
      Tmp = [Tmp temp]; % Tmp keeps growing, forming a matrix of 1*cols
      temp = 0;
    end
    %
    [Max, Index] = max(Tmp);% .
    n_rows = Index + rows2 − 1;% New column of output image.
    Fimg = [ ];
    Fimg = F(1:Index,:);% First image is pasted till Index.
    Fimg(Index:n_rows,:) = S((Index:n_rows)−Index+1,:);%Second image is pasted after Index.
    imwrite(Fimg,sprintf(‘comb%d.tif’,ni))
  end
  figure
  imshow(Fimg)
  imwrite(Fimg,‘comb.tif’)
  clear image
  cd (oldpath);
clc;clear all;close all;
path=‘/Users/mostafa/Documents/school/2020-2021/lab stuff/All Pics/exp13/3/timelapse/’;
readPath=dir(fullfile(path,‘**/*.tif’));
[~,idx] = sort([readPath.datenum]);
readPath= readPath(idx);
FPS=30;
name=‘dapi’
vidname=sprintf(‘%s’, name);%uses the name of the file that contained the data as a video file
name with extension .mp4
vidObj=VideoWriter(vidname,‘MPEG-4’); %creates the video file and videos object
vidObj.FrameRate=FPS;%sets frame rate
open(vidObj)
figure(46)
for i=1:length(readPath)
  image=imread(fullfile(readPath(i).folder,readPath(i).name));
  imshow(image);
  vidFrame=getframe(gcf);
  writeVideo(vidObj,vidFrame)
end
close(vidObj);
```

### Appendix E. MATLAB Videomaking Script

```
clc;clear all ;close all;
path=‘C:\Users\abdel\OneDrive\Desktop\dapiredo\cotrol\timelapse’;
cd(path)
readPath=dir(fullfile(path,‘**/*.tif’));
for i=1:length(readPath)
  image(i).orig=(imread(fullfile(path,readPath(i).name)));
  image(i).orig(image(i).orig>=250)=0;
  image(i).bw=bwareafilt(imbinarize((image(i).orig(:,:,3))),[30,10000]);
  image(i).s = regionprops(image(i).bw,‘centroid’);
    image(i).centroids = cat(1, image(i).s.Centroid);
end
save(‘data.mat’)
```

### Appendix F. MATLAB Migration Analysis Script

clc;clear all;close all;
path=‘C:\Users\abdel\OneDrive\Desktop\dapiredo\exp3\timelapse’;
cd(path)
load(‘data.mat’);
image(1).ord=image(1).centroids;
for i=2:length(image);
[image(i).D,I] = pdist2(image(i).centroids,image(i−1).ord,‘euclidean’,‘Smallest’,1);
image(i).ord=image(i).centroids(I,:);
y=image(i−1).ord(:,2) − image(i).ord(:,2);
x= − image(i).ord(:,1);
image(i).angle=atan2d(y,x);
end
save(‘data2.mat’)

### Appendix G. MATLAB K-Means Segmentation Function

```
function [bw3] = kseg(p)
%KSEG Takes an input of a microscope image and returns a binary mask
of where cells are located
% Detailed explanation goes here
I = im2gray(p);
RGB=I;
wavelength = 2.\hat{ }(0:5) * 3;
orientation = 0:45:135;
g = gabor(wavelength,orientation);
%I = rgb2gray(im2single(i));
gabormag = imgaborfilt(I,g);
% montage(gabormag,‘Size’,[4 6])
for i = 1:length(g)
  sigma = 0.5*g(i).Wavelength;
  gabormag(:,:,i) = imgaussfilt(gabormag(:,:,i),1*sigma);
end
% montage(gabormag,‘Size’,[4 6])
nrows = size(RGB,1);
ncols = size(RGB,2);
[X,Y] = meshgrid(1:ncols,1:nrows);
featureSet = cat(3,I,gabormag,X,Y);
L2 = imsegkmeans(featureSet,6,‘NormalizeInput’,true);
[i2]=detectCells(p);
L9=L2(i2);
% figure
% imshow(i2)
h11=mode(L9);
h10=L9 ~= h11;
L10=L9(h10);
h12=mode(L10);
h13=L10 ~= h12;
L11=L10(h13);
h14=mode(L11);
L5=L2==h11;
% imshow(L5)
L6=L2==h12;
L8=L2==h14;
if (size(L10)/size(L9))< 0.05
  L7=L5;
else
  L7=L6+L5;
end
L7=L7==1;
bw3=activecontour(I,L7,‘edge’);
end
```

### Appendix H. MATLAB Strain Analysis Script

```
clc;clear all;close all;
pathr(1).ns=‘C:\Users\abdel\OneDrive\Desktop\Strainim\s1\ns\comb.tif’;
pathr(1).s=‘C:\Users\abdel\OneDrive\Desktop\Strainim\s1\s\comb.tif’;
pathr(1).sp=‘C:\Users\abdel\OneDrive\Desktop\Strainim\s1’;
pathr(2).ns=‘C:\Users\abdel\OneDrive\Desktop\Strainim\s2\ns\comb.tif’;
pathr(2).s=‘C:\Users\abdel\OneDrive\Desktop\Strainim\s2\s\comb.tif’;
pathr(2).sp=‘C:\Users\abdel\OneDrive\Desktop\Strainim\s2’;
pathr(3).ns=‘C:\Users\abdel\OneDrive\Desktop\Strainim\s3\ns\comb.tif’;
pathr(3).s=‘C:\Users\abdel\OneDrive\Desktop\Strainim\s3\s\comb.tif’;
pathr(3).sp=‘C:\Users\abdel\OneDrive\Desktop\Strainim\s3’;
pathr(4).ns=‘C:\Users\abdel\OneDrive\Desktop\Strainim\s2\ns\comb.tif’;
pathr(4).s=‘C:\Users\abdel\OneDrive\Desktop\Strainim\s2\ns\comb.tif’;
pathr(4).sp=‘C:\Users\abdel\OneDrive\Desktop\Strainim’;
ranv=[1 2 3 4];
while length(ranv)>0
  h4=randi(length(ranv));
  rn=ranv(h4);
  ranv(h4)=[ ];
  I=imread(pathr(rn).ns);
  I2=imread(pathr(rn).s);
  if randi(2)==2
    figure, imshow(I), hold on
    for n=1:25
      [x(n,:),y(n,:)]=ginput(4);
    end
    plot(x′,y′)
    figure, imshow(I2), hold on
    for n=1:25
      [x1(n,:),y1(n,:)]=ginput(4);
    end
    plot(x1′,y1′)
  else
    figure, imshow(I2), hold on
    for n=1:25
      [x1(n,:),y1(n,:)]=ginput(4);
    end
    plot(x1′,y1′)
    figure, imshow(I), hold on
    for n=1:2
      [x(n,:),y(n,:)]=ginput(4);
    end
    plot(x′,y′)
  end
  [rows,columns]=find(I==255);
  scle=max(columns);
  topd=sqrt((x(:,2)−x(:,3)).\hat{ }2 + (y(:,2)−y(:,3)).\hat{ }2);
  topde=sqrt((x1(:,2)−x1(:,3)).\hat{ }2 + (y1(:,2)−y1(:,3)).\hat{ }2);
  botd=sqrt((x(:,1)−x(:,4)).\hat{ }2 + (y(:,1)−y(:,4)).\hat{ }2);
  botde=sqrt((x1(:,1)−x1(:,4)).\hat{ }2 + (y1(:,1)−y1(:,4)).\hat{ }2);
  atopd=mean(topd)
  atopde=mean(topde)
  abotd=mean(botd)
  abotde=mean(botde)
  stopd=std(topd)
  stopde=std(topde)
  sbotd=std(botd)
  sbotde=std(botde)
  distop=atopde-atopd
  disbot=abotde-abotd
  cd(pathr(rn).sp)
  save(‘data5.mat’)
end
```

### Appendix I. MATLAB Strain Statistics Script

clc; clear all;
d1=load(‘C:\Users\abdel\OneDrive\Desktop\Strainim\s1\data3.mat’);
d2=load(‘C:\Users\abdel\OneDrive\Desktop\Strainim\s2\data3.mat’);
d3=load(‘C:\Users\abdel\OneDrive\Desktop\Strainim\s3\data3.mat’);
dn=load(‘C:\Users\abdel\OneDrive\Desktop\Strainim\s3\dataneg.mat’);
ht1=ttest(d1.topd,d1.topde)
ht2=ttest(d2.topd,d2.topde)
ht3=ttest(d3.topd,d3.topde)
htn=ttest(dn.topd,dn.topde)
hb1=ttest(d1.botd,d1.botde)
hb2=ttest(d2.botd,d2.botde)
hb3=ttest(d3.botd,d3.botde)
hbn=ttest(dn.botd,dn.botde)
con=400/d1.scle;
e1str=(d1.topde−d1.topd)./d1.topd;
e2str=(d2.topde−d2.topd)./d2.topd;
e3str=(d3.topde−d3.topd)./d3.topd;
e1bstr=(d1.botde−d1.botd)./d1.botd;
e2bstr=(d2.botde−d2.botd)./d2.botd;
e3bstr=(d3.botde−d3.botd)./d3.botd;
cstr=(dn.topde−dn.topd)./dn.topd;
cbstr=(dn.botde−dn.botd)./dn.botd;
[p,tbl,stats] = anova1([e1str e2str e3str cstr ] );
figure
multcompare(stats)
[p1,tbl1,stats1] = anova1([e1bstr e2bstr e3bstr cbstr]);
figure
multcompare(stats1)

### Appendix J. MATLAB Migration Statistics Script

%% loading data
clc;clear all;
e1=load(‘C:\Users\abdel\OneDrive\Desktop\dapiredo\ex1\timelapse\data2.mat’);
e2=load(‘C:\Users\abdel\OneDrive\Desktop\dapiredo\ex2\timelapse\data2.mat’);
e3=load(‘C:\Users\abdel\OneDrive\Desktop\dapiredo\exp3\timelapse\data2.mat’);
c=load(‘C:\Users\abdel\OneDrive\Desktop\dapiredo\cotrol\timelapse\data2.mat’);
%% stats
sf=400/425;
[e1.Df]=pdist2(e1.image(61).ord,e1.image(1).ord,‘euclidean’,‘Smallest’,1)*sf;
y=e1.image(1).ord(:,2) − e1.image(61).ord(:,2);
x=e1.image(1).ord(:,1) − e1.image(61).ord(:,1);
e1.af=atan2d(y,x);
h1=find(e1.Df>=(3*mean(e1.Df)));
e1.Df(h1)=[ ];
e1.af(h1)=[ ];
[c.Df] = pdist2(c.image(61).ord,c.image(1).ord,‘euclidean’,‘Smallest’,1)*sf;
y=c.image(1).ord(:,2) − c.image(61).ord(:,2);
x=c.image(1).ord(:,1) − c.image(61).ord(:,1);
c.af=atan2d(y,x);
h1=find(c.Df>=(3*mean(c.Df)));
c.Df(h1)=[ ];
c.af(h1)=[ ];
[e2.Df] = pdist2(e2.image(61).ord,e2.image(1).ord,‘euclidean’,‘Smallest’,1)*sf;
y=e2.image(1).ord(:,2) − e2.image(61).ord(:,2);
x=e2.image(1).ord(:,1) − e2.image(61).ord(:,1);
e2.af=atan2d(y,x);
h1=find(e2.Df>=(3*mean(e2.Df)));
e2.Df(h1)=[ ];
e2.af(h1)=[ ];
[e3.Df] = pdist2(e3.image(61).ord,e3.image(1).ord,‘euclidean’,‘Smallest’,1)*sf;
y=e3.image(1).ord(:,2) − e3.image(61).ord(:,2);
x=e3.image(1).ord(:,1) − e3.image(61).ord(:,1);
e3.af=atan2d(y,x)
h1=find(e3.Df>=(3*mean(e3.Df)));
e3.Df(h1)=[ ];
e3.af(h1)=[ ];
y = [e1.af′, e2.af′, e3.af′, c.af′ ];
group = repelem(1:4, 1, [numel(e1.af),numel(e2.af),numel(e3.af),numel(c.af)]);
[p,tbl,stats] = anova1(y,group);
figure
multcompare(stats)
y1 = [e1.Df, e2.Df, e3.Df, c.Df ];
group1 = repelem(1:4, 1, [numel(e1.af),numel(e2.af),numel(e3.af),numel(c.af)]);
[p1,tbl1,stats1]= anova1(y1,group1);
figure
multcompare(stats1)

### Appendix K. MATLAB Variomap Script

```
clc;clear all; close all;
%%
import mlreportgen.ppt.*;
slidesFile = ‘Actin process';
slides = Presentation(slidesFile);
presentationTitleSlide = add(slides,‘Title Slide’);
replace(presentationTitleSlide,‘Title’,‘slidesFile’);
path=‘C:\Users\abdel\OneDrive\Desktop\6-17 3t3\comb2’;
readPath=dir(fullfile(path,‘**/*.tif’));
for i=1:length(readPath)
  P=imread(fullfile(readPath((i)).folder,readPath((i)).name));
  figure
  imshow(P)
  I=im2gray(P);
  slidingmean=conv2(I,ones(9)/81,‘same’);
  slidingstd=stdfilt(I);
  T = graythresh(I)
  h65=I*0;
  h65(I>= (slidingmean+ slidingstd) & im2double(I)>=T )=255;
  bw=imbinarize(h65)*1;
  bw=I;
  bw=imbinarize(bw);
  figure
  imshow(bw)
  fti=fft2(bw);
  % fti2=abs(fti).\hat{ }2;
  Zc = conj(fti);
  fti2=fti.*Zc;
  fti3=ifft2(fti2);
  var=sum((sum((bw−mean(mean(bw))).\hat{ }2)));
  fti4=fftshift(fti3)/var;
  fti5=fti4(end:−1:1,:);
  figure
  contour(fti5)
  saveas(gcf,sprintf(‘temp%f.png’,i));%saves figure as .pgn file
  temppic=imread(sprintf(‘temp%f.png’,i));
  figure
  montage({P,temppic})
  saveas(gcf,sprintf(‘tem%f.png’,i));%saves figure as .pgn file
  plot1=Picture(sprintf(‘tem%f.png’,i));%imports picture
  pictureSlide = add(slides,‘Title and Content’);%adds new slide
  replace(pictureSlide,‘Title’,readPath((i)).name);
  contents = find(pictureSlide,‘Content’);% Finds the text box in the presentation to replace with
picture
  replace(contents(1),plot1);%sets picture to slide
  [h7 h8]=size(bw);
  xl=h7/2;
  yl=h8/2;
  xa=h7/8;
  ya=yl/8;
  % ylim([xl−xa xl+xa])
  % xlim([yl−ya yl+ya])
end
close(slides);%closes powerpoint
for i=1:length(readPath)
  delete(sprintf(‘temp%f.png’,i))
  delete(sprintf(‘tem%f.png’,i))%delets all the temperary pictures
end
% cd (oldFolder)%returns to old directory
```

### Appendix L. MATLAB Detect Cells Function

function BWfinal = detectcell(I)
% DETECTCELLS detects cells using image segmention techniques.
I=im2gray(I);
I(I==255)=(mean(mean(I)));% removing scale bar
I(I==0)=mean(mean(I));%removing scale bar
[~,threshold] = edge(I,’prewitt’);%getting threshold
fudgeFactor = .5;
BWs = edge(I,‘prewitt’,threshold*fudgeFactor);%detecting edges using
half the threshold as normal
se90 = strel(‘line’,3,90);
se0 = strel(‘line’,3,0);
BWsdil = imdilate(BWs,[se90 se0]);%dialating image
BWdfill = imfill(BWsdil,‘holes’);
BWnobord = BWdfill;
seD = strel(‘diamond’,1);
BWfinal = imerode(BWnobord,seD);
BWfinal = imerode(BWfinal,seD);
end

### Appendix L. NRVM Isolation (Preliminary Studies Prior to Using Adult Cardiac Fibroblasts)

### Cell Selection (NRVM)

### NRVM Isolation

NRVMs were prepared from six, 1-3 day old pups, as previously described, with minor modifications. The University of Colorado Denver Animal Care and Use Committee's regulations were fulfilled for all animal studies [133]. Using scissors, the ventricles and atria were separated, and the ventricles were then separated, dissociated in calcium-free and bicarbonate-free Hanks with Hepes (CBFHH) buffer containing Heparin (Sigma-Aldrich) (10 U·mL-1), and digested in a CBFHH solution containing 1.12 mg mL-1 of trypsine (Gibco) and 20 μg·mL-1 of DNAse (Sigma-Aldrich). Two sequential pre-plating steps, on 100 mm dishes in Dulbecco's modified Eagle's medium (Gibco), were used to enrich cardiomyocytes (>90% purity) over non-myocytes [133]. First, 4.5 g augmented with 5% bovine calf serum (Gibco) and then 2 mg mL-1 vitamin B12 (Sigma-Aldrich) and cultured unattached cells, predominantly myocytes, were harvested and cultured. Finally, 42,000 cells were seeded onto previously prepared gelatin-fibronectin-coated beams. The following day, the old media was aspirated, and the beams were submerged in fresh media and allowed to adhere further to the beam until the next day.

### Appendix M. Antibodies Used in this Study

| | Reference | | | | Reference | | |
|---|---|---|---|---|---|---|---|
| Primary | for | | | | for | | |
| Antibody | Primary | Concentration | Animal/Color | Secondary | Secondary | Concentration | Solvent |
| Invitrogen | A12381 | 1:500 | N/A | N/A | N/A | 1:500 | BSA |
| Alexa Fluor | | | Red | (conjugate) | (conjugate) | | |
| 594 | | | | | | | |
| Phalloidin | | | | | | | |
| (Plalloidin) | | | | | | | |
| Sigma- | A2547 | 1:200 | Mouse | Invitrogen | A21202 | 1:500 | BSA |
| Alrich | | | Green | Alexa Fluor | | | |
| Monoclonal | | | | 488 anti-mouse | | | |
| Anti-Actinin | | | | | | | |
| α-Smooth | | | | | | | |
| Muscle | | | | | | | |
| (SMA) | | | | | | | |
| Invitrogen | MA5-13356 | 1:50 | Mouse | Invitrogen | A21202 | 1:500 | BSA |
| Paxillin | | | Green | Alexa Fluor | | | |
| Monoclonal | | | | 488 anti-mouse | | | |
| Antibody | | | | | | | |
| (Paxillin) | | | | | | | |
| Proteintech | 15939-1-AP | 1:200 | Rabbit | Anti-Rabbit | T6778-1ML | 1:500 | BSA |
| Piezo1 | | | Red | TRITC | | | |
| Polyclonal | | | | antibody | | | |
| antibody | | | | | | | |
| (Piezo1) | | | | | | | |
| N/A | N/A | N/A | Blue | ThermoFisher | 62247 | 1:8000 | PBS |
| | | | | 4′,6-diamidino- | | | |
| | | | | 2-phenylindole, | | | |
| | | | | dihydrochloride | | | |
| | | | | (DAPI) | | | |

### Appendix N. Example ImageJ Macro Code for Paxillin Quantification

run(“Split Channels”);
selectWindow(“C3-1.lsm”);
close( );
run(“RGB Color”);
run(“RGB Stack”);
selectWindow(“C1-1.lsm”);
selectWindow(“C2-1.lsm”);
run(“Next Slice [>]”);
setAutoThreshold(“Default dark”);
//run(“Threshold...”);
setOption(“BlackBackground”, false);
run(“Convert to Mask”, “method=Default background=Dark calculate”);
run(“Measure”);

### Appendix O. Matlab Script for SMA Intensity

close all;clear;clc
%Retrieves the File
imageOrig = imread(‘1.tif‘); % Place the name of the file in the pink part
%creates a copy of the file
imgAna = imageOrig; % (so that we are not editing the original)
% shows the copy
Figure(‘Name’,’Original Figure’) %creates a figure window
Imshow(imgAna); % print image (for grayscale images)
% Average Intensity of Image
% Download package to run function mean2
avgIntens = mean2(imgAna) %average intensity of the image
%Normalizing Value
numCells = 5; %number of cells in image
normIntens = avgIntens/numCells; %normalized value

### Appendix P. Macro Code ImageJ for SMA Piezo1

run(“Split Channels”);
close( );
run(“RGB Color”);
run(“RGB Stack”);
setAutoThreshold(“Default dark”);
//run(“Threshold...”);
setOption(“BlackBackground”, false);
run(“Convert to Mask”, “method=Default background=Dark calculate”);
run(“Measure”)

1. A cell stressor device analyzing a biomechanical response of cells or tissues, the cell stressor device comprising:
a body comprising a pair of opposing ledges, wherein:
a bottom of each ledge of the pair of ledges is attached to a bar;
a first side of a first ledge of the pair of ledges faces a first side of a second ledge of the pair of ledges; and
a second side of the first ledge faces away from a second side of the second ledge, and
wherein the pair of ledges and the bar form a window having an opening facing away from a top of the body;
a bender shaped and dimensioned to be inserted into the window, wherein a length (Lb) and a height (Hb) of the bender are less than a length (Lw) and a height (Hw) of the window;
a beam shaped and dimensioned to be inserted into the window between the bender and the pair of ledges to contact the pair of ledges; and
means for moving the bender alternately away from and back toward the bar along an axis (A) to cause a top of the bender to impinge upon a bottom of the beam.

2. The cell stressor device of claim 1, wherein at least a portion of a top facing surface of the beam (TFSB) is configured for culturing of one or more mammalian cell types; and a width of the device (Wd) is sufficient to submerge the at least a portion of the TFSB in a cell culture medium.

3. (canceled)

4. The cell stressor device of claim 2, wherein at least a portion of each ledge is arcuately shaped and dimensioned to facilitate submerging the at least a portion of the TFSB in a container holding the cell culture medium.

5. The cell stressor device of claim 1, wherein Lb is less than or equal to a length of the opening (Lo).

6-8. (canceled)

9. The cell stressor device of claim 1, wherein each ledge of the pair of ledges is L-shaped and each ledge comprises a long leg and a short leg.

10. (canceled)

11. The cell stressor device of claim 9, wherein;
each ledge of the pair of ledges comprises a shelf formed on or coupled to a bottom facing surface of the short leg and coupled to a window facing surface of the long leg; and
the shelf extends partially along the window facing surface of the long leg for a first distance less than the Hw.

12. (canceled)

13. The cell stressor device of claim 11, wherein the shelf extends partially along the bottom facing surface of the short leg for a second distance less than a second distance of the short leg.

14. The cell stressor device of claim 1, wherein:
each ledge comprises a shelf; and
when inserted into the window, two portions (P) of a back facing surface of the beam contact a front facing surface (S) of the shelf of each ledge.

15. (canceled)

16. The cell stressor device of claim 1, wherein Hw is less than or equal to a height of the beam (HB).

17-18. (canceled)

19. The cell stressor device of claim 1, wherein the cell stressor device further comprises a screw, and the bar comprises a bore axially formed therethrough to receive the screw, wherein the bore and the screw comprise matching threads to facilitate securely receiving the screw and moving the bender alternatively away from and back toward the bar along the axis.

20-24. (canceled)

25. The cell stressor device of claim 1, wherein the means for moving is configured to cause the beam to undergo both compression and tension.

26. The cell stressor device of claim 1, wherein the means for moving comprises at least one of a motor, a screw, a torque and/or a force sensor, or a motor operably coupled to a screw.

27. The cell stressor device of claim 1, wherein one or more of the body, the bender, the beam, and the means for moving is fabricated at least in part using a 3D printing process.

28. A system for analyzing a biomechanical response of cells or tissues, the system comprising a plurality of cell stressor devices according to claim 1.

29. A method for analyzing a biomechanical response of cells or tissues using a cell stressor device, the method comprising:
a providing step comprising providing a cell stressor device comprising:
a body comprising a pair of opposing ledges, wherein a bottom of each ledge of the pair of ledges is attached to a bar;
a window formed by the pair of ledges and the bar, wherein the window comprises an opening facing away from a top of the body;
a bender shaped and dimensioned to be inserted into the window; and
a beam shaped and dimensioned to be inserted into the window between the bender and the pair of ledges to contact the pair of ledges;
a culturing step comprising culturing cells on the beam submerged in the cell culture media;
a moving step comprising moving the bender alternately away from and back toward the bar along an axis (A) to cause a top of the bender to impinge on the beam; and
an identifying step comprising identifying biophysical effect(s) of the moving step.

30. The method of claim 29, wherein the culturing step comprises culturing cells of a tissue on at least a portion of a top facing surface of the beam submerged in the cell culture media.

31. (canceled)

32. The method of claim 29, further comprising imaging the cells to facilitate identifying the biophysical effect(s) of the moving step, wherein imaging the cells comprises:
generating a first image of the cells prior to the moving step;
generating a second image of the cells after the mobbing step; and
identifying the biophysical effect(s) of the moving step from the first image and the second image.

33-34. (canceled)

35. The method of claim 29, further comprising:
sterilizing at least one of the body, the window, the beam, or the bender of the cell stressor device; and
assembling the cell stressor device prior to the culturing step.

36-37. (canceled)

38. The method of claim 29, further comprising causing, by the moving step, the cultured cells to undergo at least one of: compression stress, tension stress, and torsion stress.

39. The method of claim 29, wherein the moving step comprises causing the beam to undergo at least one of: compression stress, tension stress, and torsion stress.
